(19) United States
(12) Patent Application Publication
(10) Pub. No.: US 2025/0282878 A1
(43) Pub. Date: Sep. 11, 2025

(54) COMPOSITIONS AND METHODS FOR MODULATING ANTIGEN BINDING ACTIVITY

(71) Applicant: Oregon Health & Science University, Portland, OR (US)

(72) Inventors: Stuart Ibsen, Portland, OR (US); Michael Brasino, Portland, OR (US)

(73) Assignee: Oregon Health & Science University, Portland, OR (US)

(21) Appl. No.: 18/861,178

(22) PCT Filed: Apr. 27, 2023
(86) PCT No.: PCT/US23/66322

§ 371 Date: Oct. 28, 2024

Related U.S. Application Data
(60) Provisional application No. 63/336,174, filed on Apr. 28, 2022.

(51) Int. Cl. C07K 16/28; A61K 39/00; A61P 35/00; C07K 16/44; G01N 33/68

(52) U.S. Cl. CPC C07K 16/2863; A61P 35/00; C07K 16/44; G01N 33/6854; A61K 2039/505; C07K 2317/24; C07K 2317/92; C07K 2319/50

(57) ABSTRACT

The present disclosure describes compositions, kits and methods for immunoglobulin blocking constructs that may be crosslinked to immunoglobulins to form blocked immunoglobulin complexes useful for selectively modulating the native binding activity of immunoglobulins and enhancing their utility as research tools and therapeutic treatments. Further, wherein a blocking construct for modulating binding activity of an antigen binding domain is disclosed.

### CROSS-REFERENCE TO RELATED APPLICATIONS

This application is the 371 National Phase of International Application No. PCT/US23/66322, filed on Apr. 27, 2023, which claims priority to and the benefit of the earlier filing of U.S. Provisional Application No. 63/336,174, filed on Apr. 28, 2022, each of which is incorporated by reference herein in its entirety.

### INCORPORATION BY REFERENCE OF SEQUENCE LISTING

A computer readable text file, entitled “0046-0086US_SeqList.xml” created on or about Oct. 28, 2024, with a file size of 143,360 bytes, contains the Sequence Listing for this application and is hereby incorporated by reference in its entirety.

### FIELD OF THE DISCLOSURE

The present disclosure relates generally to modifying the native binding activity of immunoglobulins, including antibodies. More specifically, the field involves engineering immunoglobulin blocking constructs that may be crosslinked to immunoglobulins to form blocked immunoglobulin complexes useful for selectively modulating the native binding activity of immunoglobulins and enhancing their utility as research tools and therapeutic treatments.

### BACKGROUND OF THE DISCLOSURE

Monoclonal antibody technology has revolutionized biotechnology and medicine. Despite the development of various alternative binding proteins, such as nanobodies, affibodies and alpha bodies, antibodies are still widely used affinity agents both as research tools and for therapeutic applications. Antibodies are naturally in an active state (or “on-state”) in that they are generally capable of binding to their targets and this capacity cannot be temporally or spatially controlled under normal physiological conditions. For cancer immunotherapy applications where the antibodies induce immune activation by blocking regulatory signaling proteins on the surface of immune cells, this creates a challenge. The infused therapeutic antibodies in their active state cause the antibodies to bind not just to immune cells in the region where they would be effective, such as tumor draining lymph nodes, but also throughout the body, resulting in life threatening side effects for subjects in need of therapy. Moreover, the complex structure of monoclonal antibodies has historically complicated their structural modification and functionalization. So, while the idea of activatable antibodies has been investigated previously, past approaches have had challenges. For example, protease activated antibodies were recently realized through the genetic fusion of two interacting capping peptides at the N-termini of both the antibody heavy and light chains (Trang et al., Nature Biotechnology 1, 2019 doi: 10.1038/s41587-019-0135-x). The resulting antibody's binding was blocked until a protease cleaved these capping peptides, allowing the antibody to activate and bind its target. This capping strategy called for the genetic modification of the antibody sequence, requiring monoclonal antibody expression outside the means of most laboratories. Further, many site-specific labeling methods claim to have site specificity, such as maleimide modification of sulfhydryl groups (cysteines) or N-hydroxy-succinimide modification of amine groups (lysines), but they frequently label each IgG a variable number of times at multiple locations across the 4 protein chains (two light and two heavy). To address this, skilled persons have taken IgG binding proteins, such as Protein G and A, which bind at specific locations on the heavy chain of IgGs outside of the antigen binding region, and modified them to covalently attach to the IgG at those sites, providing a site-specific conjugation handle. These binding proteins have allowed the conjugation of drug payloads for targeted therapy, and dyes or imaging agents for immunostaining. However, these attachment sites are not located in proximity to the antigen binding site of the antibody as would be ideal to attach blocking moieties.

Thus, the ability to control antibody binding activity, such as changing the antibody from a nonbinding “off-state” to a binding “on-state” with spatial and temporal control would be useful for localizing antibody binding in therapeutic applications and in various other biological applications and assays. Moreover, an ideal binding protein would consistently bind to a specific attachment site located in proximity to the antigen binding site.

However, none these key features are present in clinically available antibodies. Moreover anchoring binding proteins to an antibody with an irreversible bond remains a challenge; binding proteins including a reactive group that consistently bind to a specific attachment site in proximity to the antigen binding site of an antibody are not commercially available.

### SUMMARY OF THE DISCLOSURE

The disclosed materials and methods relate to blocking constructs that are useful modulating the binding activity of immunoglobulins, such as anti-FLAG antibody and cetuximab. Some of the disclosed embodiments use crosslinker kappa light chain-binding domains for crosslinking blocking constructs to antigen binding domains. In an accordance with an embodiment, a kappa light chain-binding polypeptide, includes a set of one or more crosslinker kappa light chain-binding domains, in which a crosslinker kappa light chain-binding domain in the set includes a Protein L amino acid sequence. In some embodiments, at least one amino acid residue in the Protein L amino acid sequence is substituted by a photo-reactive crosslinker residue having an activation wavelength.

Also provided is a therapeutic blocked immunoglobulin complex, including a therapeutic immunoglobulin including a kappa light chain; and a set of one or more blocking constructs, the blocking constructs in the set including a crosslinker kappa light chain-binding domain including a Protein L amino acid sequence in which at least one amino acid residue in the Protein L amino acid sequence is substituted by a photo-reactive crosslinker residue to crosslink the therapeutic immunoglobulin to the blocking constructs. In some embodiments, the blocking constructs are photo-cleavable. In other embodiments, the blocking constructs are enzymatically cleavable.

For instance, one provided embodiment is a kappa light chain-binding polypeptide, which polypeptide includes: a set of one or more crosslinker kappa light chain-binding domains, in which a crosslinker kappa light chain-binding domain in the set includes a Protein L amino acid sequence in which at least one amino acid residue in the Protein L amino acid sequence is substituted by a crosslinker residue, such as a photo-reactive crosslinker residue having an activation wavelength.

Another embodiment is a blocking construct for modulating binding activity of an antigen binding domain, the blocking construct including: a kappa light chain-binding polypeptide including a Protein L amino acid sequence in which at least one amino acid residue in the Protein L amino acid sequence is substituted by a photo-reactive crosslinker residue having an activation wavelength; da blocking moiety including an epitope configured to bind competitively to an antigen binding site of the antigen binding domain; and a flexible tether, operatively connecting the kappa light chain-binding polypeptide to the blocking moiety.

Also provided are embodiments of blocking constructs for modulating the binding activity of an antigen binding domain, wherein the blocking construct includes: a kappa light chain-binding polypeptide of any of the embodiments herein; which is operatively connected via a flexible tether to a blocking moiety configured to bind to antigen binding site of the antigen binding domain.

Another embodiment is a blocking construct for modulating the binding activity of an antigen binding domain, the blocking construct including: a kappa light chain-binding polypeptide including: a set of one or more crosslinker kappa light chain-binding domains, in which a crosslinker kappa light chain-binding domain in the set includes a Protein L amino acid sequence in which at least one amino acid residue in the Protein L amino acid sequence is substituted by a photo-reactive crosslinker residue having an activation wavelength; which is operatively connected via a flexible tether to a blocking moiety that is configured to bind to antigen binding site of the antigen binding domain.

Embodiments also include blocked immunoglobulin complexes including: an immunoglobulin crosslinked to a set of one or more blocking constructs.

Example blocked immunoglobulin complexes include: a heavy chain including SEQ ID NO: 42; and a light chain including SEQ ID NO: 43, which is crosslinked to the blocking construct of any of the herein described embodiments.

Pharmaceutical compositions that include at least one blocked immunoglobulin complex of any of the described embodiments are provided.

Also provided are method embodiments, including methods of treating cancer that include administering (to a subject in need thereof) a therapeutically effective amount of a blocked immunoglobulin complex of one of the herein described embodiments. One of ordinary skill in the art would know how to determine a subject is in need of such administration.

Another provided method is for modifying the binding activity of an antigen binding domain, the method including: providing a set of one or more blocking constructs as described herein; and crosslinking the set of one or more blocking constructs to an antigen binding domain to thereby modify the binding activity of the antigen binding domain.

Methods for producing a kappa light chain-binding polypeptide are also provided, which methods include: expressing a nucleic acid sequence encoding a kappa light chain-binding polypeptide amino acid sequence of a kappa light chain-binding polypeptide of any of the embodiments described herein, in transformant cells, to produce the kappa light chain-binding polypeptide; and extracting and purifying the produced kappa light chain-binding polypeptide from the transformant cells.

Another embodiment is a method for producing a blocking construct, the method including: expressing a nucleic acid sequence encoding the amino acid sequence of a blocking construct of any of the embodiments described herein in transformant cells to produce the blocking construct; and extracting and purifying the produced blocking construct from the transformant cells.

Yet another provided method is a method for producing a blocked immunoglobulin complex, including: expressing a nucleic acid sequence encoding the amino acid sequence of the immunoglobulin of a blocked immunoglobulin complex of any of the herein described embodiments in transformant cells to produce the immunoglobulin; expressing a nucleic acid sequence encoding the amino acid sequence of a blocking construct of any of the herein described embodiments in transformant cells to produce the blocking construct; extracting and purifying the immunoglobulin and the blocking construct from the respective transformant cells; and exposing the immunoglobulin and blocking constructs to a crosslinker trigger to crosslink the immunoglobulin to the blocking constructs and thereby produce blocked immunoglobulin complex.

83. A method for researching the binding activity of an immunoglobulin, including: selecting a immunoglobulin; crosslinking to the immunoglobulin a blocking construct selected from the blocking construct of any of embodiments 1, 2, 31-56, or 58; and measuring the binding activity of the immunoglobulin.

There are also provided kits for use in any of the methods describe herein. Exemplary kits are for use in modifying the binding activity of an antigen binding domain, which kits include two or more components selected from: a kappa light chain-binding polypeptide as described herein; a blocking construct of any of the embodiments described herein 8; a blocked immunoglobulin complex of any of the embodiments as described herein; and a pharmaceutical composition as described herein.

### BRIEF DESCRIPTION OF THE DRAWINGS

Some of the drawings submitted herein may be better understood in color. Applicant considers color versions of the drawings (which were included in priority Application No. 63/336,174, filed on Apr. 28, 2022, and/or were published in Brasino et al., Comm. Biol. 5, Art. 1357, 2022 (doi.org/10.1038/s42003-022-04094-1) as part of the original submission and reserves the right to present color images of the drawings in later proceedings

FIG. 1A shows a graphical representation of a blocking construct including a kappa light chain-binding polypeptide operatively connected to blocking moiety by a flexible tether, the flexible tether includes a set of one or more triglycine linkers and a set of one or more alpha helix motifs; FIG. 1B shows the blocking construct of FIG. 1A, in which the kappa light chain-binding polypeptide of the blocking construct is cross crosslinked to a kappa light chain of an antigen binding domain and in which the blocking moiety includes an epitope configured to competitively bind to an antigen binding site of the antigen binding domain.

FIGS. 2A and 2B show graphical representations of a blocking construct crosslinked to an antigen binding domain and including a flexible tether configured to bend and have sufficient end-to-end length to present a blocking moiety to an antigen binding site of the antigen binding domain.

FIGS. 3A and 3B show graphical representations of a blocking constructs crosslinked to an antigen binding domain and including, respectively, a fully rigid and a fully flexible tether.

FIG. 4 shows a graphical representation of a blocking constructs crosslinked to an antigen binding domain having multiple end-to-end length radii.

FIGS. 5A and 5B provide graphical representations of blocking constructs with kappa light chain-binding polypeptide and IgG isotype antibody. FIG. 5A shows a graphical representations of a set of one or more blocking constructs including a kappa light chain-binding polypeptide that, when in the proximity of the kappa light chains of the antigen binding domains of an IgG isotype antibody, have a binding interaction with the kappa light chains and thereby form non-covalent bonds between the blocking constructs and the antigen binding domains; and, FIG. 5B shows a graphical representation of the blocking constructs and IgG isotype antibody of FIG. 5A forming non-covalent bonds that, upon exposure to light of the activation wavelength of the photo-reactive crosslinker residues of the kappa light chain-binding domains of the blocking constructs, activates the photo-reactive crosslinker residues and crosslinks the kappa light chain-binding domains to the kappa light chains and thereby forms covalent bonds between the blocking constructs and antigen binding domains.

FIG. 6 is a graphical representation of an IgG Isotype immunoglobulin (such as cetuximab or other anti-EGFR IgG isotype antibodies) crosslinked to a set of one or more blocking constructs to form a blocked immunoglobulin complex.

FIGS. 7A-7C illustrate antibody inactivation through PpL-based attachment of a tethered blocking peptide. FIG. 7A shows a schematic of an antibody activation strategy; FIG. 7B is a graphical rendering of the crystal structure (PDB 1MHH) of Protein L (PpL) bound to a Fab fragment of an IgG isotype antibody and shows a graphical representation of a flexible linker having from N-terminus to C-Terminus a structure represented by the polypeptide formula: (G₂S)-EA₃K)₄-G₂S-(EA₂K)₄-(G₂S); and FIG. 7C is a line graph showing a Protein L (PpL) linked to a blocking moiety or was shown to block an anti-FLAG antibody better than the blocking moiety alone.

FIGS. 8A-8C show successful photoconjugation of PpL to an antibody light chain, and successful blocking of an anti-FLAG antibody. FIG. 8A shows that different locations were chosen on PpL based on the crystal structure (PB 1 mhh) to introduce BpA. FIG. 8B is an image of a reducing SDS PAGE gel with 50 μM of each PpL irradiated with 1 μM mouse IgG1 kappa antibody showing different locations chosen on Protein L to introduce the non-canonical photo-reactive crosslinker amino acid residue Benzoyl-4-Phenylalanine, in which a photo-crosslinked product is shown between the kappa light chain of the IgG1 kappa antibody and the PpL with a R33Bpa mutation only. FIG. 8C is a graph showing each amino acid in PpL with the solvent exposure level and number of antibody carbons that are within 1 nm. The higher the solvent exposure and the higher the number of proximal carbons the more likely the amino acid was to be a successful candidate for modification with BpA. Encircled symbols indicate which amino acids were modified; amino acid R33 was ultimately successful with a high surface exposure and number of proximal carbons. The amino acids are labeled as “Free” or “Bound” based on a 0.35 nm distance cut-off between the PpL sidechain and antibody atoms.

FIGS. 9A and 9B are images of reducing SDS PAGE gels showing, respectively, 100 μM of PpLR33BpA (R33) with 4 μM mouse IgG1 kappa antibody (Ab) irradiated under 360 nm light for the time indicated, and the R33 mutant fused to the flexible linker of FIG. 7B and crosslinked to an anti-FLAG antibody and then operatively connected enzymatically to a blocking moiety including a photo-cleavable linker.

FIG. 10 is a graph showing the binding activity of anti-FLAG antibody alone modified with a blocking moiety including a photocleavable linker after photoirradiation for the indicated time (n=3). The first four bars show data from antibody with linker; the last two bars are from antibody (without linker).

FIG. 11 is a line graph comparing cetuximab affinity for EGFR after combining cetuximab antibody with, respectively, a EGFR blocking construct including an R33 mutant of a PpL (C-PpL-E) kappa light chain-binding polypeptide photoconjugated (i.e., crosslinked) to a cetuximab antibody, and an EGFR blocking construct including a wild type PpL (cetuximab plus PpL-E), in which the addition of two molar excess PpL-E kappa light chain binding polypeptide has no significant effect on cetuximab binding affinity suggesting that photoconjugating the PpL kappa light chain-binding polypeptide to the cetuximab antibody facilitates establishing an effective concentration of a blocking construct at an antigen binding site.

FIG. 12 is a line graph showing that a chymotrypsin treatment had no detectable effect on the binding affinity of cetuximab itself, nor did the photoconjugation of a blocking construct lacking a blocking moiety including an EGFR epitope.

FIG. 13 is a line graph and an inset image, the inset image is a reducing SDS PAGE gel and ELISA (n=3) showing cetuximab alone vs. cetuximab photoconjugated to a PpL-R33BpA blocking construct including a chymotrypsin cleavable linker (C-PpL-X-E), with and without protease treatment with chymotrypsin, and the line graph shows that the cetuximab photoconjugated to the PpL-R33BpA blocking construct had about a 9-fold lower EGFR binding affinity compared to the EGFR binding affinity of cetuximab alone.

FIG. 14 is a line graph and inset image, the inset image is a reducing SDS PAGE gel and ELISA (n=3) of a cetuximab, a cetuximab photoconjugated to a blocking construct lacking a blocking moiety including an EGFR epitope (C-PpL-No), and a cetuximab photo-conjugated to a blocking construct including an EGFR epitope with and without 10 minutes of light exposure, the line graph shows that the blocking construct including an EGFR epitope had a decreased affinity for EGFR.

### REFERENCE TO SEQUENCE LISTING

The nucleic acid and/or amino acid sequences described herein are shown using standard letter abbreviations, as defined in 37 C.F.R. § 1.822. One strand of each nucleic acid sequence is shown; the complementary strand is understood as included in embodiments where appropriate. In the Sequence Listing:

SEQ ID NO: 1 is a Protein L amino acid sequence of an exemplary PpL domain C*.

SEQ ID NO: 2 is a Protein L amino acid sequence of an exemplary PpL domain C₁.

SEQ ID NO: 3 is a Protein L amino acid sequence of an exemplary PpL domain C₂.

SEQ ID NO: 4 is a Protein L amino acid sequence of an exemplary PpL domain C₃.

SEQ ID NO: 5 is a Protein L amino acid sequence of an exemplary PpL Domain C₄.

SEQ ID NO: 6 is a Protein L amino acid sequence of an exemplary PpL Domain B₁.

SEQ ID NO: 7 is a Protein L amino acid sequence of an exemplary PpL Domain B₂.

SEQ ID NO: 8 is a Protein L amino acid sequence of an exemplary PpL Domain B₃.

SEQ ID NO: 9 is a Protein L amino acid sequence of an exemplary PpL Domain B₄.

SEQ ID NO: 10 is a Protein L amino acid sequence of an exemplary PpL Domain B₅.

SEQ ID NO: 11 is a Protein L amino acid sequence of an exemplary PpL protein (Uniprot Entry: Q51918).

SEQ ID NO: 12 is a Protein L amino acid sequence of an exemplary PpL domain C*, in which Xaa is a photo-reactive crosslinker residue.

SEQ ID NO: 13 is a Protein L amino acid sequence of an exemplary PpL domain C₁, in which Xaa is a photo-reactive crosslinker residue.

SEQ ID NO: 14 is a Protein L amino acid sequence of an exemplary PpL domain C₂, in which Xaa is a photo-reactive crosslinker residue.

SEQ ID NO: 15 is a Protein L amino acid sequence of an exemplary PpL domain C₃, in which Xaa is a photo-reactive crosslinker residue.

SEQ ID NO: 16 is a Protein L amino acid sequence of an exemplary PpL Domain C₄, in which Xaa is a photo-reactive crosslinker residue.

SEQ ID NO: 17 is a Protein L amino acid sequence of an exemplary PpL Domain B₁, in which Xaa is a photo-reactive crosslinker residue.

SEQ ID NO: 18 is a Protein L amino acid sequence of an exemplary PpL Domain B₂, in which Xaa is a photo-reactive crosslinker residue.

SEQ ID NO: 19 is a Protein L amino acid sequence of an exemplary PpL Domain B₃, in which Xaa is a photo-reactive crosslinker residue.

SEQ ID NO: 20 is a Protein L amino acid sequence of an exemplary PpL Domain B₄, in which Xaa is a photo-reactive crosslinker residue.

SEQ ID NO: 21 is a Protein L amino acid sequence of an exemplary PpL Domain B₅, in which Xaa is a photo-reactive crosslinker residue.

SEQ ID NO: 22 is a Protein L amino acid sequence of an exemplary PpL protein (Uniprot Entry: Q51918), in which Xaa is a photo-reactive crosslinker residue.

SEQ ID NO: 23 is an amino acid sequence of an exemplary crosslinker alpha helix motif, in which Xaa is a photo-reactive crosslinker residue.

SEQ ID NO: 24 is an amino acid sequence of an exemplary crosslinker alpha helix motif, in which Xaa is a photo-reactive crosslinker residue.

SEQ ID NO: 25 is an amino acid sequence of an exemplary crosslinker alpha helix motif, in which Xaa is a photo-reactive crosslinker residue.

SEQ ID NO: 26 is an amino acid sequence of an exemplary crosslinker alpha helix motif, in which Xaa is a photo-reactive crosslinker residue.

SEQ ID NO: 27 is an amino acid sequence of an exemplary crosslinker alpha helix motif, in which Xaa is a photo-reactive crosslinker residue.

SEQ ID NO: 28 is an amino acid sequence of an exemplary crosslinker alpha helix motif, in which Xaa is a photo-reactive crosslinker residue.

SEQ ID NO: 29 is an amino acid sequence of an exemplary crosslinker alpha helix motif, in which Xaa is a photo-reactive crosslinker residue.

SEQ ID NO: 30 is an amino acid sequence of an exemplary crosslinker alpha helix motif, in which Xaa is substituted by a photo-reactive crosslinker residue.

SEQ ID NO: 31 is an amino acid sequence of an exemplary FLAG epitope.

SEQ ID NO: 32 is an amino acid sequence of an exemplary EGFR epitope.

SEQ ID NO: 33 is an amino acid sequence of an exemplary sortase recognition site.

SEQ ID NO: 34 is an amino acid sequence of an exemplary polypeptide flexible linker consisting of the amino acid sequence in which X is a Sortase A recognition site including the amino acid sequence SEQ ID NO: 33; SEQ ID NO: 34 can be illustrated as follows: (G₄S)-(EA₃K)₄-(G₄S)-(EA₃K)₄-(G₄S)-(X), where X is a Sortase A recognition site.

SEQ ID NO: 35 is an amino acid sequence of an exemplary polypeptide flexible linker, which can be illustrated as follows: (G₄S)-(EA₃K)₄-(G₄S)-(EA₃K)₄-(G₄S).

SEQ ID NO: 36 is an amino acid sequence of an exemplary polypeptide flexible linker consisting of the amino acid sequence of SEQ ID NO: 38, in which X is a Sortase A recognition site including the amino acid sequence of SEQ ID NO: 33; SEQ ID NO: 34 can be illustrated as follows: (G₂S)-(EA₃K)₄-(G₂S)-(EA₃K)₄-(G₂S)-(X), where X is a Sortase A recognition site.

SEQ ID NO: 37 is an amino acid sequence of an exemplary polypeptide flexible linker, which can be illustrated as follows: (G₂S)-(EA₃K)₄-(G₂S)-(EA₃K)₄-(G₂S).

SEQ ID NO: 38 is an exemplary nucleic acid sequence encoding the PpL domain C* of SEQ ID NO: 1.

SEQ ID NO: 39 is an exemplary nucleic acid sequence encoding the PpL domain C₁ of SEQ ID NO: 2.

SEQ ID NO: 40 is an exemplary nucleic acid sequence encoding the PpL domain C₂ of SEQ ID NO: 3.

SEQ ID NO: 41 is an exemplary nucleic acid sequence encoding the PpL domain C₃ of SEQ ID NO: 4.

SEQ ID NO: 42 is an exemplary nucleic acid sequence encoding the PpL Domain C₄ of SEQ ID NO: 5.

SEQ ID NO: 43 is an exemplary nucleic acid sequence encoding the PpL Domain B₁ of SEQ ID NO: 6.

SEQ ID NO: 44 is an exemplary nucleic acid sequence encoding the PpL Domain B₂ of SEQ ID NO: 7.

SEQ ID NO: 45 is an exemplary nucleic acid sequence encoding the PpL Domain B₃ of SEQ ID NO: 8.

SEQ ID NO: 46 is an exemplary nucleic acid sequence encoding the PpL Domain B₄ of SEQ ID NO: 9.

SEQ ID NO: 47 is an exemplary nucleic acid sequence encoding the PpL Domain B₅ of SEQ ID NO: 10.

SEQ ID NO: 48 is an exemplary nucleic acid sequence encoding the PpL protein (Uniprot Entry: Q51918) of SEQ ID NO: 11.

SEQ ID NO: 49 is an exemplary nucleic acid sequence encoding the PpL domain C* of SEQ ID NO: 12; the codon encoding the photo-reactive crosslinker residue is at positions 271-273.

SEQ ID NO: 50 is an exemplary nucleic acid sequence encoding the PpL domain C₁ of SEQ ID NO: 13; the codon encoding the photo-reactive crosslinker residue is at positions 280-282.

SEQ ID NO: 51 is an exemplary nucleic acid sequence encoding the PpL domain C₂ of SEQ ID NO: 14; the codon encoding the photo-reactive crosslinker residue is at positions 274-276.

SEQ ID NO: 52 is an exemplary nucleic acid sequence encoding the PpL domain C₃ of SEQ ID NO: 15; the codon encoding the photo-reactive crosslinker residue is 274-276.

SEQ ID NO: 53 is an exemplary nucleic acid sequence encoding the PpL Domain C₄ of SEQ ID NO: 16; the codon encoding the photo-reactive crosslinker residue is at positions 271-273.

SEQ ID NO: 54 is an exemplary nucleic acid sequence encoding the PpL Domain B₁ of SEQ ID NO: 17; the codon encoding the photo-reactive crosslinker residue is at positions 274-276.

SEQ ID NO: 55 is an exemplary nucleic acid sequence encoding the PpL Domain B₂ of SEQ ID NO: 18; the codon encoding the photo-reactive crosslinker residue is at positions 271-273.

SEQ ID NO: 56 is an exemplary nucleic acid sequence encoding the PpL Domain B₃ of SEQ ID NO: 19; the codon encoding the photo-reactive crosslinker residue is at positions 271-273.

SEQ ID NO: 57 is an exemplary nucleic acid sequence encoding the PpL Domain B₄ of SEQ ID NO: 20; the codon encoding the photo-reactive crosslinker residue is at positions 274-276.

SEQ ID NO: 58 is an exemplary nucleic acid sequence encoding the PpL Domain B₅ of SEQ ID NO: 21; the codon encoding the photo-reactive crosslinker residue is at positions 277-279.

SEQ ID NO: 59 is an exemplary nucleic acid sequence encoding the PpL protein (Uniprot Entry: Q51918) of SEQ ID NO: 22; the codon encoding the photo-reactive crosslinker residue is at positions 4234-4236.

SEQ ID NO: 60 is an exemplary nucleic acid sequence encoding the crosslinker alpha helix motif of SEQ ID NO: 23; the codon encoding the photo-reactive crosslinker residue is at positions 73-75.

SEQ ID NO: 61 is an exemplary nucleic acid sequence encoding the crosslinker alpha helix motif of SEQ ID NO: 24; the codon encoding the photo-reactive crosslinker residue is at positions 82-85.

SEQ ID NO: 62 is an exemplary nucleic acid sequence encoding the crosslinker alpha helix motif of SEQ ID NO: 25; the codon encoding the photo-reactive crosslinker residue is at positions 76-78.

SEQ ID NO: 63 is an exemplary nucleic acid sequence encoding the crosslinker alpha helix motif of SEQ ID NO: 26; the codon encoding the photo-reactive crosslinker residue is at positions 76-78.

SEQ ID NO: 64 is an exemplary nucleic acid sequence encoding the crosslinker alpha helix motif of SEQ ID NO: 27; the codon encoding the photo-reactive crosslinker residue is at position 76-78.

SEQ ID NO: 65 is an exemplary nucleic acid sequence encoding the alpha helix motif of SEQ ID NO: 28; the codon encoding the photo-reactive crosslinker residue is at positions 73-75.

SEQ ID NO: 66 is an exemplary nucleic acid sequence encoding the crosslinker alpha helix motif of SEQ ID NO: 29; the codon encoding the photo-reactive crosslinker residue is at positions 76-78.

SEQ ID NO: 67 is an exemplary nucleic acid sequence encoding the crosslinker alpha helix motif of SEQ ID NO: 30; the codon encoding the photo-reactive crosslinker residue is at positions 76-78.

SEQ ID NO: 68 is an exemplary nucleic acid sequence encoding the FLAG epitope of SEQ ID NO: 31.

SEQ ID NO: 69 is an exemplary nucleic acid sequence encoding the EGFR epitope of SEQ ID NO: 32.

SEQ ID NO: 70 is an exemplary nucleic acid sequence encoding the sortase recognition SEQ ID NO: 33.

SEQ ID NO: 71 is an exemplary nucleic acid sequence encoding the polypeptide flexible linker SEQ ID NO: 34; the codons encoding the Sortase A recognition site are at positions 487-501.

SEQ ID NO: 72 is an exemplary nucleic acid sequence encoding the polypeptide flexible linker SEQ ID NO: 35.

SEQ ID NO: 73 is an exemplary nucleic acid sequence encoding the polypeptide flexible linker of SEQ ID NO: 36; the codons encoding the Sortase A recognition site are at positions 439-453.

SEQ ID NO: 74 is an exemplary nucleic acid sequence encoding the polypeptide flexible linker of SEQ ID NO: 37.

SEQ ID NO: 75 is an amino acid sequence of an exemplary cetuximab heavy chain.

SEQ ID NO: 76 is an amino acid sequence of an exemplary cetuximab light chain.

SEQ ID NO: 77 is an amino acid sequence of an exemplary cetuximab heavy chain.

SEQ ID NO: 78 is an amino acid sequence of an exemplary cetuximab light chain.

SEQ ID NO: 79 is an exemplary nucleic acid sequence encoding the cetuximab heavy chain of SEQ ID NO: 75.

SEQ ID NO: 80 is an exemplary nucleic acid sequence encoding the cetuximab light chain of SEQ ID NO: 76.

SEQ ID NO: 81 is an exemplary nucleic acid sequence encoding the cetuximab heavy chain of SEQ ID NO: 77.

SEQ ID NO: 82 is an exemplary nucleic acid sequence encoding the cetuximab light chain of SEQ ID NO: 78.

SEQ ID NO: 83 is the amino acid sequence ((GGS)₂) of a linker portion.

SEQ ID NO: 84 is the amino acid sequence (GGGGS) of a linker portion.

SEQ ID NO: 85 is the amino acid sequence ((EA₃K)₄) of a rigid portion of a linker.

SEQ ID NO: 86 is the amino acid sequence (SSSSS, (S₅)) of a linker portion.

SEQ ID NO: 87 is the amino acid sequence (EA₃K) of a linker portion.

SEQ ID NOs: 88-90 are amino acid sequences of representative peptide cleavage sites.

### DETAILED DESCRIPTION

As used herein, “(2R)-2-amino-3-fluoro-3-(4-((2-nitrobenzyl)oxy) phenyl) propanoic acid” (FnbY) refers to a photo-reactive crosslinker residue that is activated for crosslinking through exposure to ultraviolet light of 302 nm or 365 nm wavelength to form a reactive quinone methide which selectively forms covalent bonds with Cys, Lys, His, Tyr, Trp, Met, Arg, Asn, and Gln (Liu et al., J Am Chem Soc, 141(24): 9458-9462, 2020).

As used herein, “4-{4-[1-(9-Fluorenylmethyloxycarbonylamino)ethyl]-2-methoxy-5-nitrophenoxy}butanoic acid” refers to a fluorenylmethoxycarbonyl protecting group (Fmoc) (CAS 162827-98-7) known by skilled persons to be useful as a photocleavable linker.

As used herein, “activation” refers to rendering molecules capable of reaction or to increase the reactivity of substrate molecules by the presence of other molecules, moieties, motifs, domains, or functional groups proximal to the substrate molecules.

As used herein, “amino acid sequence” refers to the order of amino acids as they occur in a polypeptide. Unless otherwise stated, skilled persons will understand that the order of an amino acid sequence forming a polypeptide is written from the N-terminus to the C-terminus of the polypeptide.

As used herein, “antibody” refers to a polypeptide produced by an immune system that has binding activity to a specific antigen (Kapingidza et al., Subcell Biochem. 94:465-497, 2020). For example, an antibody includes an immunoglobulin or any fragment thereof and may include immunoglobulins of any class such as IgG, IgA, IgD, IgE, IgM and their respective subclasses and any mutants of the immunoglobulins and fragments. Furthermore, an antibody may include a recombinant antibody such as a single chain variable fragment (scFV), a chimeric antibody such as a humanized antibody, an antibody complex, or any other immunoglobulin modification product including an antigen recognition site. Furthermore, an antibody fragment according to the present specification may be a fragment of an antibody including an antigen recognition site or a fragment of an antibody that does not include an antigen recognition site. Examples of the fragment of an antibody that does not include an antigen recognition site include a protein including the Fc region only of an immunoglobulin, an Fc fusion protein, and any mutants and modification products thereof. Examples of antibodies include, but are not limited to, fragment antigen binding (Fab), Fv, scFv, and Fd fragments, chimeric antibodies, humanized antibodies, single-chain antibodies (scAb), single domain antibodies (dAb), single domain heavy chain antibodies, a single domain light chain antibodies, bi-specific antibodies, multi-specific antibodies, and fusion proteins including an antigen-binding (also referred to herein as antigen binding) portion of an antibody and a non-antibody protein. The antibodies, in some cases, are further conjugated to other moieties, such as members of specific binding pairs, e.g., biotin (member of biotin-avidin specific binding pair), and the like. The antibodies, in some cases, are bound to a solid support, including, but not limited to, polystyrene plates or beads, and the like. Also encompassed by the term are Fab′, Fv, F(ab′)₂, and or other antigen binding fragments that retain specific binding to antigen, and monoclonal antibodies. Example antibodies are monovalent or bivalent. An antibody, in some instances, is an Ig monomer, which is a “Y-shaped” molecule that includes four polypeptide chains: two heavy chains and two light chains, connected by disulfide bonds.

As used herein, “anti-Flag antibody” refers to an antibody configured to bind to a FLAG peptide or epitope. For example, as provided herein, SEQ ID NO: 31 sets forth the amino acid sequence of an exemplary FLAG epitope (also known as a FLAG-tag). FLAG peptide is well known in the art to be useful as a protein tag that can be readily incorporated into an engineered polypeptide using recombinant molecular methods as those disclosed herein. Skilled persons will understand that FLAG peptide is highly specific and is thus useful for protein purification by affinity chromatography as well as for tagging proteins in vivo. FLAG-based protein purification is comparatively mild relative to other purification methods in the art and is thus known to be well suited for isolating proteins or protein complexes including multiple subunits since it generally does not disrupt such complexes. anti-Flag antibody is available commercially from a number of vendors (e.g., Monoclonal Anti-Flag M2 antibody from Sigma-Aldrich, Inc., St. Louis, MO; Cat. Nos.: F1804 and F3165).

As used herein, “antigen binding domain” refers to the functional domain of an immunoglobulin that binds to a specific antigen or epitope. For example, a fragment antigen-binding (Fab) fragment is an antigen binding domain including one light chain and one heavy chain in which each chain includes one constant domain and one variable domain.

As used herein, “antigen binding site,” “antibody binding pocket,” “antibody binding site,” or “paratope” may be used interchangeably and collectively refer to the portion of an antibody binding domain composed of the variable domains of both the light and heavy chains that bind to an epitope. For example, skilled persons will understand that conventional IgG isotype immunoglobulins are composed of six complementarity-determining regions (CDRs) located in the V_{H} and V_{L} domains. Thus, antibody fragments such as Fab and Fv may be viewed as autonomous units containing a single, complete antigen binding site for antigen recognition, as evidenced by the 1:1 stoichiometry between antigen and Fab (or Fv) conserved among the antibody structures and isotypes known in the art (Porter, Nature 182(4636): 670-671, 1958).

As used herein, “antigen” refers to any molecule or molecular structure that binds to a specific immunoglobulin or antibody. Antigens often trigger an immune response from an immune system and may include toxins, chemicals, viruses, bacteria, proteins, nucleic acids, and lipids.

As used herein, “to bind” and its verb conjugates refer to the reversible or non-reversible attachment of one molecule to another. For example, as disclosed herein, a PpL domain C* crosslinker kappa light chain-binding polypeptide (SEQ ID NO: 1) was engineered to reversibly bind to a kappa light chain of an antibody with a dissociation constant of 130 nM (Graille et al., Structure 9:679-687, 2001). In a further example, the amino acid residue corresponding to position 33 of the amino acid sequence of the PpL domain C* kappa light chain-binding polypeptide (SEQ ID NO: 1) was substituted by a noncanonical photo-reactive amino acid residue: 4-benzoyl phenylalanine (BpA), to form a crosslinker kappa light chain-binding domain capable of forming a covalent bond between the crosslinker kappa light chain-binding domain and a kappa light chain by photo-crosslinking.

As used herein, “binding activity” and “binding affinity” may be used interchangeably and collectively refer to the strength of the binding interaction between a molecule and its ligand. For example, in some instances “binding activity” and “binding affinity” collectively refer to the strength of a polypeptide's binding interaction between another polypeptide, or fragment or domain thereof. Binding affinity is typically measured and reported by the equilibrium dissociation constant (KD), which is used to evaluate and rank order strengths of binding interactions. The binding affinity and dissociation constants can be measured quantitatively. Methods for determining binding affinities are well known to the skilled person and can be selected, for instance, from the following methods: surface plasmon resonance (SPR), enzyme-linked immunosorbent assay (ELISA), kinetic exclusion analysis (KinExA assay), Bio-layer interferometry (BLI), flow cytometry, fluorescence spectroscopy techniques, isothermal titration calorimetry (ITC), analytical ultracentrifugation, radioimmunoassay (RIA or IRMA), and enhanced chemiluminescence (ECL). Typically, a dissociation constant KD is determined at temperatures between the range of 20° C. and 30° C.

As used herein, “binding domain” refers to a functional domain of a polypeptide having a binding affinity for a specific ligand.

As used herein, “binding interaction” refers to an attractive interaction between two molecules that results in a stable association in which the molecules are in proximity to each other.

As used herein, “binding kinetics” refers to the rate at which a molecule binds to, and then dissociates, from a ligand. For example, binding kinetics may include the measurement of an “on-rate” and an “off-rate” of a molecule and its ligand.

As used herein, “blocking construct” refers to a construct configured to competitively bind with the target ligand (e.g., antigen) of an immunoglobulin.

As used herein, “blocking moiety” refers to a moiety including an epitope configured to competitively bind with the epitope of a specific antigen.

As used herein, “cleavable linker” refers to a linker configured to cleave upon its activation by a trigger.

As used herein, “cetuximab,” “IMC-C₂₂₅,” and “Erbitux®” may be used interchangeably and collectively refer to a recombinant chimeric monoclonal antibody that binds to the extracellular domain of the human epidermal growth factor receptor (EFGR) and is clinically approved by the FDA for treatment of non-small cell carcinoma, metastatic colon cancer, or head and neck squamous cell carcinoma (HNSCC). The amino acid sequence of an exemplary cetuximab heavy chain is the amino acid sequence as set forth in SEQ ID NO: 75 or SEQ ID NO: 77. The amino acid sequence of an exemplary cetuximab light chain is the amino acid sequence as set forth in SEQ ID NO: 76 or SEQ ID NO: 78. SEQ ID NO: 79 is an exemplary nucleic acid sequence encoding the cetuximab heavy chain of SEQ ID NO: 75. SEQ ID NO: 80 is an exemplary nucleic acid sequence encoding the cetuximab light chain of SEQ ID NO: 76. SEQ ID NO: 81 is an exemplary nucleic acid sequence encoding the cetuximab heavy chain of SEQ ID NO: 77. SEQ ID NO: 82 is an exemplary nucleic acid sequence encoding the cetuximab light chain of SEQ ID NO: 78.

As used herein, “to competitively bind” and its verb conjugates refer to the dynamic of a first ligand inhibiting the binding interaction of a second ligand to a molecule. In some instances, the competitive binding of a first ligand to a molecule decreases the on-rate binding kinetics of a second ligand to the molecule. For example, as disclosed herein, a blocking moiety including the amino acid sequence set forth in SEQ ID NO: 32 (known by skilled persons to transiently block the binding interaction between cetuximab and epidermal growth factor receptor (EGFR)) was operatively connected to the C-terminal end of a flexible tether (SEQ ID NO: 36) and attached to a crosslinker kappa light chain polypeptide including the amino acid sequence set forth in in SEQ ID NO: 1 (or interchangeably, SEQ ID NO: 12) to competitively inhibit the native binding activity of cetuximab to EFGR and thereby modulate EGFR's on-rate with cetuximab.

As used herein, “configured” refers to the selective arrangement, form, or order of a composition of matter.

As used herein, “conservative variant” refers to a variant including a conservative amino acid substitution. “Conservative” amino acid substitutions are those substitutions that do not substantially affect or decrease a function of a protein, such as the ability of the protein to induce an immune response when administered to a subject. The term conservative variation also includes the use of a substituted amino acid in place of an unsubstituted parent amino acid. Furthermore, one of ordinary skill will recognize that individual substitutions, deletions or additions which alter, add or delete a single amino acid or a small percentage of amino acids (for instance less than 5%, in some embodiments less than 1%) in an encoded sequence are conservative variations where the alterations result in the substitution of an amino acid with a chemically similar amino acid.

As used herein, “construct” refers to a composition of matter formed, made, or created by combining parts or elements.

As used herein, “contour length” refers to distance between the two ends of a polymer chain in a configuration in which the polymer chain is at its maximum physically possible extension.

As used herein, “covalent bond” refers to a chemical bond involving the sharing of electron pairs between atoms.

As used herein, “to crosslink” and its verb conjugates refers to forming covalent bonds or relatively short sequences of chemical bonds to join two molecules together. Crosslinking reagents or (i.e., crosslinkers) are molecules that include a reactive group or residue capable of chemically attaching, for example, to the specific functional groups (such as primary amines, sulfhydryls, or carbonyls) on proteins. Examples of crosslinking chemistry include photo-reactive groups that become reactive when exposed to light such as diaziriness, aryl azides, and phenylazides.

As used herein, “crosslinker” refers to a molecule that includes a reactive group or residue capable of chemically attaching to the specific functional groups of other molecules, such as proteins. In some embodiments the reactive group is a photo-reactive group. In some embodiments, the photo-reactive group may be an aryl azide or any of its derivatives. In other embodiments the photo-reactive group may be a phenyl azide, or any of its derivatives. In further embodiments, photo-reactive group may be a diazirine or any of its derivatives. Skilled persons will understand that crosslinker reactive groups are selected on the basis of their chemical reactivities (i.e., specificity for particular functional groups) and other chemical properties that affect their behavior in different applications. For example, four protein functional group targets: primary amines (—NH₂), carboxyls (—COOH), sulfhydryls (—SH), and carbonyls (—COOH) are generally relied upon by skilled persons for crosslinking to a protein.

As used herein, “domain” refers to a distinct functional and/or structural unit of a polypeptide. For example, the C4 domain of wildtype Protein L is known by skilled persons as an Ig-binding domain because of its binding affinity for immunoglobulin (Kastern et al., J. Biol. Chem. 267(18): 12820-12825, 1992). Moreover, skilled persons will understand that a domain may be any portion of a polypeptide that is self-stabilizing and folds into its tertiary structure independently from the rest of the polypeptide.

As used herein, “effective concentration” or “effective molarity” may be used interchangeably and collectively refer to the ratio of the equilibrium constants for two equivalent binding interactions, where one occurs intramolecularly and one occurs intermolecularly. For example, for an intramolecular interaction, the encounter rate between a tethered domain equals the rate of the same untethered interaction at a given concentration (i.e., “the effective concentration) (Sorensen et. al, Proc. Natl. Acad. Sci. U.S.A., 119(14): e2114397119, 2022; Krishnamurhty et al. J. Am. Chem. Soc.; 129(5): 1312-1320, 2007). In a further example, as disclosed herein, blocking constructs including blocking moieties were operatively connected to antibodies by a flexible tether to facilitate a tethered intramolecular binding interaction between the epitopes of the blocking moieties with their respective antigen binding sites and thereby increase the effective concentration of the blocking moieties at the antigen binding sites.

As used herein, “epitope” refers to the part of an antigen to which an antibody attaches or binds itself. Skilled persons will understand that antibodies may reversibly bind to an epitope presented by an antigen through non-covalent interactions which include hydrogen, ionic, hydrophobic, and Van der Waals bonding.

As used herein, “end-to-end length” or “displacement length” may be used interchangeably and collectively refer to the distance between the two ends of a polymer chain in a particular configuration. For example, skilled persons will understand that the displacement length of a coiled polymer, when fully extended and in an uncoiled configuration (i.e., at its “contour length”) will exceed the displacement length of the coiled polymer in a coiled configuration.

As used herein, “exposure of [a thing] to light” refers to an amount of light of reaching the thing. In some instances, an exposure may be measured as the amount of light reaching a specific unit area. For example, skilled persons will understand that the International System of Units (SI) derived unit of illuminance, lux (lx), equals one lumen per square meter. As disclosed herein, exposure may be measured in units of mW/cm²

As used herein, “Fc region” and “Fc domain” may be used interchangeably. Skilled persons will understand that a Fc region is the tail region of an immunoglobulin that interacts for example with cell surface receptors called Fc receptors. Thus, the Fc region or Fc domain means the Fc region or Fc domain of an immunoglobulin or antibody. In various embodiments, the Fc region is from a mammalian IgG (antibody), including human IgG, mouse IgG, rat IgG, goat IgG, bovine IgG, guinea pig IgG, and rabbit IgG. The Fc region may also be from human IgM or human IgA. In various embodiments, the Fc region is from a human IgG (antibody), such as from a human IgGi (antibody), human IgG2 (antibody), human IgG4 (antibody), or from a human IgGi (antibody).

As used herein, “flexible tether” refers to a tether configured to bend. For example, a bending flexible tether facilitates a first joined molecule to move at a distance that is less than the movement radius between the first and second joined molecules bound by an inflexible tether of the same length. In some instances, the ability to bend may be measured as a flexural modulus or bend modulus (i.e., the ability of a material to bend). For example, skilled persons will understand that the International System of Units (SI) of flexural modulus is the pascal (Pa, or N/m²). In some embodiments, a flexible tether includes a set of one or more Gly-Gly-Ser linkers. Skilled persons will understand that the stiffness of a flexible tether including a flexible polypeptide linker may be tuned by selectively configuring the length and overall glycine content of the flexible polypeptide linker; moreover, skilled persons will understand that the persistence length of a flexible polypeptide linker may be selectively configured to increase the effective concentration of intramolecular interaction partners (Rosmalen et al., Biochemistry, 56; 6565-6574, 2017).

As used herein, “fusion protein” and “fusion polypeptide” may be used interchangeably and collectively refer to a protein including a first protein joined to a second protein. A fusion protein is created through joining of two or more amino acid sequences that originally coded for separate proteins. Thus, a fusion protein may include a multimer of identical or different proteins which are expressed as a single, linear polypeptide.

As used herein, “immunoglobulin binding polypeptide” refers to a protein having binding activity to an immunoglobulin (or an antibody or a fragment of an antibody). An example of an immunoglobulin binding polypeptide includes, a kappa light chain binding polypeptide that binds to the antigen binding domain of immunoglobulin.

As used herein, “immunoglobulin” or “immunoglobulins” refer to any class of polypeptide present in an immune system or modified or derived from a polypeptide native to an immune system which functions as an antibody.

As used herein, “intramolecular interaction” refers to an interaction between two covalently bound molecules.

As used herein, “intermolecular interaction” refers to an interaction between two or more molecules not covalently bound to each other.

As used herein, “irreversible bond” refers to a chemical bond having a sufficiently high enough activation energy to not to react in a context.

As used herein, “kappa light chain” refers to a kappa light chain isotype and any variant, fragment, or fusion protein thereof. Skilled persons will understand that a light chain includes a light chain variable domain (V_{L}) and a light chain constant domain (C_{L}) and (in humans) may be called kappa (κ) or lambda (λ), based on the polypeptide sequence of its constant domain (Townsend et al., Front Immunol. 7:388, 2016).

As used herein, “kappa light chain-binding polypeptide” and “kappa light chain-binding protein” may be used interchangeably and collectively refer to a polypeptide or protein having a binding affinity to a kappa light chain of an antigen binding domain and includes any variant, fragment, or fusion protein thereof that maintains its kappa light chain binding affinity. For example, skilled persons will understand that a kappa light chain-binding polypeptide is capable of binding to a subclass 1, 3 or 4 kappa light chain of an antibody (also called V_{Ki}, V_{Km} and V_{Kiv}, as in Nilson et al., J. Biol. Chem. 267:2234-2239, 1992). In some embodiments, a kappa light chain-polypeptide includes Protein L and any variant, fragment or fusion protein thereof that has maintained the binding property.

As used herein, “kappa light chain-binding domain” refers to a functional domain of a kappa light chain-binding polypeptide that binds to a kappa light chain. For example, skilled persons will understand that Protein L (PpL) includes various kappa light chain-binding domains, known in the art to specifically bind to a kappa light chain. Examples of Protein L kappa light chain-binding domains are known in the art and include, without limitation: a PpL domain C*, a PpL domain C₁, a PpL domain C₂, a PpL domain C₃, a PpL domain C₄, a PpL domain B₁, a PpL domain B₂, a PpL domain B₃, a PpL domain B₄, and a PpL domain B₅ (Graille et al., Structure 9:679-687, 2001). PpL Domains C1 through C4 are from PpL3316 strain (Murphy et al., Mol Microbiol 12(6): 911-920, 1994). PpL Domains B₁ through B₅ are from PpL312 strain (Kastern et al., J. Biol. Chem. 267(18): 12820-12825, 1992). See also WO 2016/096643.

In some embodiments, a kappa light chain-binding domain may be configured as a crosslinker, i.e., to include a reactive group or residue(s) capable of chemically attaching to the specific functional group(s) of other molecules and thereby form a crosslinker kappa light chain-binding domain. In some embodiments, the reactive group is a photo-reactive group, enabling the crosslinker kappa light chain-binding domain to crosslink to a ligand (e.g., an antibody kappa light chain) upon exposure to light of its activation wavelength. In some embodiments, the activation wavelength of the photo-reactive group of the crosslinker kappa light chain-binding domain is wavelength in the ultraviolet range, such as 365 nm.

As used herein, “ligand” refers to a molecule that binds to another molecule.

As used herein, “linker” refers to a molecule that covalently joins at least two other molecules.

As used herein, “moiety” refers to one of a part or portion of a molecule into which the molecule is divided. For example, skilled persons understand that a hemoglobin molecule includes four heme moieties.

As used herein, “molecule” refers to one or more atoms bound to together, representing the smallest unit of a compound that can take part in a chemical reaction.

As used herein, “monoclonal antibody” refers to an antibody produced by a group of identical cells, all of which were produced from a single cell by repetitive cellular replication. That is, the clone of cells only produces a single antibody species. While a monoclonal antibody can be produced using hybridoma production technology, other production methods known to those skilled in the art can also be used (e.g., antibodies derived from antibody phage display libraries).

As used herein, “movement radius” refers to the end-to-end distance between a first joined molecule bound to a second joined molecule by a tether.

As used herein, “to modulate” and its verb conjugates refer to the act of exerting a modifying or controlling influence on a thing.

As used herein, “motif” refers to a distinctive, sometimes recurrent, pattern in the sequence (i.e., primary structure) or spatial relationship (i.e., secondary structure) of a polymer. For example, as used herein, a “tri-glycine motif” refers to a portion of a polypeptide sequence consisting of three consecutive glycine molecules. In a further example, the polypeptide sequence “LPETG” or “LPXTG” (i.e., Leu-Pro-Glu-Thr-Gly or Leu-Pro-X-Thr-Gly) is a conserved motif known by those skilled in the art as a Sortase A transamidase recognition site (Maresso & Schneewind, Pharmacological Reviews; 60:128-141, 2008).

As used herein, “native binding activity” refers to a prior or original binding activity of a molecule.

As used herein, “native” refers to a prior or original state of a thing created by either natural or artificial means. Us used herein, “prior state” and “prior configuration” refer, respectively, to any state and any configuration of a thing referred to as “native” that exists prior to the modulation of the thing by a blocking construct. For example, the native binding activity of an antibody may be the native binding activity that exists just prior to the antibody's conjugation to a blocking construct whereby, upon the blocking construct's modulation of the antibody's binding kinetics, the antibody's binding activity is modulated to a novel (i.e., non-native) binding activity. A prior configuration may or may not differ from its original configuration and may be effected by any means, natural or otherwise.

As used herein, “non-covalent bond” refers to a chemical bond involving any combination of electrostatic, hydrogen bond, van der Waals, hydrophobic, hydrophilic, or induced dipole interactions between atoms.

As used herein, “oligonucleotide” and “polynucleotide” refers to a polymer including two or more covalently-bound nucleotide molecules. For example, a polynucleotide may include a strand of two or more deoxyribonucleotide or two or more ribonucleotide molecules, or any combination of two or more deoxyribonucleotide and ribonucleotide molecules.

As used herein, “operatively connected” refers to the joining or binding of two molecules either via a linker or directly to each other.

As used herein, “p-benzoyl-L-phenylalanine” (pBpA) refers to a halogenated photo-reactive crosslinker residue that is activated for crosslinking through exposure to ultraviolet light of 350 nm to 365 nm wavelength (Joiner et al., Protein Science, 28:1163-1170, 2019).

As used herein, “p-isothiocyanate phenylalanine” (pNCSF) refers to a crosslinker residue having an isothiocyanate functional group that is activated for crosslinking through exposure to ultraviolet light including wavelengths from 350 nm to 365 nm wavelength (Martvoň et al., Chemical Papers 27.5:692-697, 1973). pNCSF forms crosslinks to proximal amine groups under mild conditions (see Xuan et al., Angew Chem Int Ed Engl 55(34): 10065-10068, 2016, doi.org/10.1002/anie.201604891). Thus, unlike other photo-activated crosslinkers discussed herein, pNCSF does not need photoactivation to crosslink proteins. It just needs the correct buffer solution, such as HEPES buffer. This can be used in alternative embodiments of the herein provided system, in which pNCSF is used to crosslink PpL (or another kappa chain binding domain) to a target antibody kappa light chain.

As used herein, “p-azidophenylalanine” (pAzF) refers to a photo-reactive crosslinker residue that is activated for crosslinking through exposure to ultraviolet light of 365 nm wavelength to form a reactive nitrene intermediate which forms covalent bonds with proximal polypeptides (Choi et al., PLOS Biol., 17(10): e3000475, 2019).

As used herein, “photo-reactive crosslinker residue” refers to a photo-activatable amino acid residue that is chemically inert in the physiological milieu, but converts into reactive groups when irradiated with light. Examples of photo-reactive crosslinker residues include 4-benzoyl-L-phenylalanine (BpA) (PubChem CID: 7020128), p-benzoyl-L-phenylalanine (pBpA), n-(Fluoroacetyl)phenylalanine (PubChem CID: 237767), p-2′-fluoroacetyl-phenylalanine (Ffact), p-vinylsulfonamido-(S)-phenylalanine, and p-isothiocyanate phenylalanine (pNCSF) (Coin, Current Opinion in Chem. Bio., 46:156-163, 2018).

As used herein, “persistence length” refers to the end-to-end length over which the direction of tangent between two ends of a polymer no longer correlate.

As used herein, “photoconjugation” refers to the binding of a first molecule to a second molecule (i.e., conjugation) by photo-reactive crosslinking.

As used herein, “polymer” refers to any of a class of natural or synthetic substances composed of two or more chemical units (e.g., “monomers”). Polymers include, for example, proteins and nucleic acids.

As used herein, “protease cleavage site” refers to an amino acid sequence that can be cleaved by a protease, such as, for example, a matrix metalloproteinase or a furin. Examples of such sites include Gly-Pro-Leu-Gly-Ile-Ala-Gly-Gln (SEQ ID NO: 88) or Ala-Val-Arg-Trp-Leu-Leu-Thr-Ala (SEQ ID NO: 89), which can be cleaved by metalloproteinases; and Arg-Arg-Arg-Arg-Arg-Arg (SEQ ID NO: 90), which can be cleaved by a furin. In therapeutic applications, the protease cleavage site can be cleaved by a protease that is produced by target cells, for example cancer cells or infected cells, or pathogens.

As used herein, “protein” and “polypeptide” may be used interchangeably and collectively refer to any polymer of two or more amino acids linked by peptide bonds and does not refer to a specific length of the product. Thus, “peptides,” “protein,” “amino acid chain,” or any other term used to refer to a chain of two or more amino acids, are included within the definition of “polypeptide,” and the term “polypeptide” may be used instead of, or interchangeably with, any of these terms. The term “polypeptide” is also intended to include products of post-translational modifications of the polypeptide like, e.g., glycosylation, which are well known in the art.

As used herein, “PubChem CID” refers to a compound ID number used as a database identifier from “PubChem,” a chemical information database administrated by the U.S. National Library of Medicine (National Center for Biotechnological Information, U.S. National Library of Medicine, 8600 Rockville Pike, Bethesda, MD 20894, USA).

As used herein, “residue” refers to single molecular unit within a polymer. For example, a residue may include, respectively, a single amino acid within a polypeptide or a single nucleotide within a polynucleotide.

As used herein, “reversible bond” refers to a chemical bond having an activation energy sufficiently low enough to react in a context. For example, the non-covalent bonding between the epitopes and antigen binding sites disclosed herein will generally have a binding kinetic off-rate of greater than zero.

As used herein, “sequence identity” refers to the similarity between two nucleic acid sequences, or two amino acid sequences. Sequence identity is frequently measured in terms of percent identity (or similarity or homology); the higher the percentage, the more similar the two sequences are. Polypeptides or domains thereof that have a significant amount of sequence identity and function the same or similarly to one another—for example, the same protein in different species—can be called “homologs.” Methods of alignment are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-244, 1988; Higgins & Sharp, Comput. Appl. Biosci. 5:151-153, 1989; Corpet et al., Nucl. Acids Res. 16:10881-90, 1988; Huang et al., Comput. Appl. Biosci. 8:155-65, 1992; and Pearson, Methods Mol. Biol. 24:307-331, 1994. Altschul et al. (J. Mol. Biol. 215:403-410, 1990) presents a detailed consideration of sequence alignment methods and homology calculations. The NCBI Basic Local Alignment Search Tool (BLAST) is available from several sources, including the National Center for Biotechnology Information (NCBI, Bethesda, MD) and on the Internet, for use in connection with the sequence analysis programs blastp, blastn, blastx, tblastn and tblastx. In a further example, methods for determining the extent of an amino acid sequence identity of an arbitrary polypeptide relative to the amino acid sequence, the SIM Local similarity program may be employed (Huang and Webb Miller, Advances in Applied Mathematics, 12:337-357, 1991), that is freely available. For multiple alignment analysis, ClustalW can be used (Thompson et al., Nucleic Acids Res., 22:4673-4680, 1994).

Nucleic acid sequences that do not show a high degree of sequence identity may nevertheless encode similar amino acid sequences, due to the degeneracy of the genetic code. Changes in nucleic acid sequence can be made using this degeneracy to produce multiple nucleic acid molecules that all encode substantially the same protein.

As used herein, “sequence” refers to a particular order in which things follow each other, such as the order of repeating molecular units in a polymer. For example, skilled persons will understand that the order of nucleic acid sequences and amino acid sequences are referred to by convention in, respectively, the order of nucleic acid units running from a 5′ end to a 3′ end and the order of amino acids running from a N-terminus to a C-terminus.

As used herein, “to substitute” and its verb conjugates refer to the substitution of any molecule with another molecule when referring to any form of chemistry or chemical concepts. For example, a substitution reaction may be a chemical reaction in which one functional group is replaced by another functional group.

As used herein, “substrate” refers to a molecule or material acted upon by another molecule or material, such as an enzyme. For example, as disclosed herein, a chymotrypsin flexible linker was configured as a chymotrypsin substrate to evaluate the effect of chymotrypsin treatment on the affinity of a blocking construct/cetuximab conjugate for EGFR.

As used herein, “tether” refers to a linker configured to limit the movement radius of a first joined molecule bound to a distal end of the linker relative to a second joined molecule bound to a proximal end of the linker.

As used herein “therapeutically effective amount” and “pharmaceutically effective amount” may be used interchangeably and collectively refer to an amount that is sufficient to effect treatment, as defined below, when administered to a subject (e.g., a mammal, such as a human) in need of such treatment. The therapeutically or pharmaceutically effective amount will vary depending upon the subject and disease condition being treated, the weight and age of the subject, the severity of the disease condition, the manner of administration and the like, which can readily be determined by one of ordinary skill in the art. For example, a “therapeutically effective amount” or a “pharmaceutically effective amount” of a compound of Formula I, or a pharmaceutically acceptable salt or co-crystal thereof, is an amount sufficient to modulate activity of EGFR-expressing cancer cells, and thereby treat a subject (e.g., a human) suffering an indication, or to ameliorate or alleviate the existing symptoms of the indication. For example, a therapeutically or pharmaceutically effective amount may be an amount sufficient to decrease a symptom of a disease or condition responsive to antibody binding of EGFR protein.

As used herein, “treatment” or “treating” refer to an approach for obtaining beneficial or desired results including clinical results. Beneficial or desired clinical results may include one or more of the following: (i) inhibiting the disease or condition (e.g., decreasing one or more symptoms resulting from the disease or condition, and/or diminishing the extent of the disease or condition); (ii) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or (iii) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival). In different embodiments, the disease or condition treated includes the cancers described herein.

As used herein, “trigger” refers to the immediate cause eliciting an effect, such as a change in configuration or an activation. For example, in some embodiments, light may be used as an activating trigger to allow the use of photocleavable flexible linkers that are resistant to enzymatic based cleavage. In other embodiments, protease or endonuclease enzyme triggers may be used to allow the use of enzymatic based triggers that are not activated by light.

In an accordance with an embodiment, disclosed are kappa light chain-binding polypeptides that include one or more crosslinker kappa light chain-binding domains (which may be referred to as a set of such domains), in which a crosslinker kappa light chain-binding domain in the set includes a Protein L amino acid sequence. The amino acid sequence of an exemplary wild type Protein L (PpL) is provided herein (SEQ ID NO: 11). In some embodiments, the kappa light chain-binding polypeptide includes a PpL engineered to be a crosslinker including a photo-reactive crosslinker residue (SEQ ID NO: 22). Skilled persons will understand that a kappa light chain binding polypeptide may include

In some examples, the kappa light chain binding polypeptides has a sequence at least 75%, 85%, 90%, 95%, 96%, 97%, 98%, or 99% identical to SEQ ID NO: 1, SEQ ID NO: 12, or any fragments thereof. For example, the kappa light chain-binding polypeptide can be a homolog or ortholog of SEQ ID NO: 1, SEQ ID NO: 12, or any fragments thereof. Exemplary sequences can be obtained using computer programs that are readily available on the world wide web and the amino acid sequences set forth herein. In some embodiments, the kappa light chain binding polypeptide has a sequence at least 75%, 85%, 90%, 95%, 96%, 97%, 98%, or 99% identical to SEQ ID NO: 2, SEQ ID NO: 13, or any fragments thereof. In some examples, the kappa light chain binding polypeptides has a sequence at least 75%, 85%, 90%, 95%, 96%, 97%, 98%, or 99% identical to SEQ ID NO: 3, SEQ ID NO: 14, or any fragments thereof. In some examples, the kappa light chain binding polypeptides has a sequence at least 75%, 85%, 90%, 95%, 96%, 97%, 98%, or 99% identical to SEQ ID NO: 4, SEQ ID NO: 15, or any fragments thereof. In some examples, the kappa light chain binding polypeptides has a sequence at least 75%, 85%, 90%, 95%, 96%, 97%, 98%, or 99% identical to SEQ ID NO: 5, SEQ ID NO: 16, or any fragments thereof. In some examples, the kappa light chain binding polypeptides has a sequence at least 75%, 85%, 90%, 95%, 96%, 97%, 98%, or 99% identical to SEQ ID NO: 6, SEQ ID NO: 17, or any fragments thereof. In some examples, the kappa light chain binding polypeptides has a sequence at least 75%, 85%, 90%, 95%, 96%, 97%, 98%, or 99% identical to SEQ ID NO: 6, SEQ ID NO: 17, or any fragments thereof. In some examples, the kappa light chain binding polypeptides has a sequence at least 75%, 85%, 90%, 95%, 96%, 97%, 98%, or 99% identical to SEQ ID NO: 7, SEQ ID NO: 18, or any fragments thereof. In some examples, the kappa light chain binding polypeptides has a sequence at least 75%, 85%, 90%, 95%, 96%, 97%, 98%, or 99% identical to SEQ ID NO: 7, SEQ ID NO: 18, or any fragments thereof. In some examples, the kappa light chain binding polypeptides has a sequence at least 75%, 85%, 90%, 95%, 96%, 97%, 98%, or 99% identical to SEQ ID NO: 8, SEQ ID NO: 19, or any fragments thereof. In some examples, the kappa light chain binding polypeptides has a sequence at least 75%, 85%, 90%, 95%, 96%, 97%, 98%, or 99% identical to SEQ ID NO: 9, SEQ ID NO: 20, or any fragments thereof. In some examples, the kappa light chain binding polypeptides has a sequence at least 75%, 85%, 90%, 95%, 96%, 97%, 98%, or 99% identical to SEQ ID NO: 10, SEQ ID NO: 21, or any fragments thereof. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Nucleic acid molecules encoding the disclosed compositions (e.g., encoding any one of (SEQ ID NOs: 38-74, or SEQ ID NOs: 79-81) and/or any homologs or variants thereof can be produced by standard approaches, such as amplification by the polymerase chain reaction (PCR). In additional embodiments, the nucleic acid molecule has a sequence at least 75%, 85%, 90%, 95%, 96%, 97%, 98%, or 99% identical to any one of SEQ ID NOs: 38-74, or SEQ ID NOs: 79-81 or a fragment thereof.

Numerous prokaryotic and eukaryotic systems are known for the expression and purification of polypeptides. For example, heterologous polypeptides can be produced in prokaryotic cells by placing a strong, regulated promoter and an efficient ribosome binding site upstream of the polypeptide-encoding 20 construct. Suitable promoter sequences include the beta-lactamase, tryptophan (trp), phage T7, and lambda PL promoters. Methods and plasmid vectors for producing heterologous proteins in bacteria or mammalian cells are described in Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene 25 Publishing Associates, 1992 (and Supplements to 2000); and Ausubel et al., Short Protocols in Molecular Biology: A Compendium of Methods from Current Protocols in Molecular Biology, 4th ed., Wiley & Sons, 1999.

Suitable prokaryotic cells for expression of large amounts of proteins include Escherichia coli and Bacillus subtilis. Often, proteins expressed at high levels are found in insoluble inclusion bodies; methods for extracting proteins from these aggregates are described for example, by Sambrook et al. (2001, see chapter 15). Recombinant expression of recombinant polypeptides in prokaryotic cells may alternatively be conveniently obtained using commercial systems designed for optimal expression and purification of fusion proteins. Such fusion proteins typically include a tag that facilitates purification. Examples of such systems include: the pMAL protein fusion and purification system (New England 35 Biolabs, Inc., Beverly, MA); the GST gene fusion system (Amersham Pharmacia Biotech, Inc., Piscataway, NJ); and the pTrcHis expression vector system (Invitrogen, Carlsbad, CA). Additional systems include the His6-tag (e.g., Roche Applied Science, Mannheim, Germany) or streptavidin binding peptide (e.g., Sigma-Aldrich, St. Louis, MO). For example, the pMAL expression system utilizes a vector that adds a maltose binding protein to the expressed protein. The fusion protein is expressed in E. coli. and the fusion protein is purified from a crude cell extract using an amylose column. If necessary, the maltose binding protein domain can be cleaved from the fusion protein by treatment with a suitable protease, such as Factor Xa. The maltose binding fragment can then be removed from the preparation by passage over a second amylose column.

The recombinant polypeptides can also be expressed in eukaryotic expression systems, including Pichia pastoris, Drosophila, Baculovirus and/or Sindbis expression systems produced by Invitrogen (Carlsbad, CA). Eukaryotic cells such as Chinese Hamster ovary (CHO), monkey kidney (COS), HeLa, Spodoptera frugiperda, and Saccharomyces cerevisiae may also be used to express recombinant polypeptides. Regulatory regions suitable for use in these cells include, for mammalian cells, viral promoters such as those from CMV, adenovirus or SV40, and for yeast cells, the promoter for 3-phosphoglycerate kinase or alcohol dehydrogenase.

The vectors can be introduced into recipient cells (such as eukaryotic cells) as pure DNA (transfection) by, for example, precipitation with calcium phosphate or strontium phosphate, electroporation, lipofection, DEAE dextran, microinjection, protoplast fusion, or microprojectile guns. Alternatively, the nucleic acid molecules can be introduced by infection with virus vectors. Systems are developed that use, for example, retroviruses, adenoviruses, or Herpes virus.

For example, a kappa light chain-binding polypeptide, blocking moiety, or blocked immunoglobulin complex (such as those described herein) produced in mammalian cells may be extracted following release of the protein into the supernatant and may be purified using an immunoaffinity column prepared using anti-MHC or other antibodies. Alternatively, the polypeptide may be expressed as a chimeric protein with, for example, β-globin. Antibody to β-globin is thereafter used to purify the chimeric protein. Corresponding protease cleavage sites engineered between the β-globin gene and the nucleic acid sequence encoding the recombinant polypeptide are then used to separate the two polypeptide fragments from one another after translation. One useful expression vector for generating β-globin chimeric proteins is pSG5 (Stratagene, La Jolla, CA).

In some embodiments, at least one amino acid residue in the sequence that binds to a target kappa light chain (e.g., a Protein L amino acid sequence) is substituted by a photo-reactive crosslinker residue having an activation wavelength.

In accordance with another embodiment, a blocking construct includes a set of one or more of crosslinker kappa light chain-binding domains in which a kappa light chain-binding domain in the set includes a Protein L amino acid sequence in which at least one amino acid residue in the Protein L amino acid sequence is substituted by a photo-reactive crosslinker residue having an activation wavelength. In some embodiments, the blocking construct includes a kappa light chain-binding polypeptide configured to have, when in the proximity of a kappa light chain of an antigen binding domain, a binding interaction with the kappa light chain and thereby form a non-covalent bond between the blocking construct and the antigen binding domain. Whereby, upon exposure of a photo-reactive crosslinker residue of a kappa light chain-binding domain of the crosslinker kappa light chain-binding polypeptide to light of its activation wavelength, the photo-reactive crosslinker residue activates and crosslinks the kappa light chain-binding domain forming the non-covalent bond to the kappa light chain and thereby forms a covalent bond between the blocking construct and antigen binding domain. In some embodiments, the blocking moiety includes an epitope configured to competitively bind to an antigen binding site of the antigen binding domain. In some embodiments, the flexible tether includes a flexible linker operatively connected at a proximal end to the kappa light chain-binding polypeptide and at a distal end to the blocking moiety. The flexible linker is configured to have an end-to-end length to tether the blocking moiety at a sufficient movement radius for the blocking moiety to establish an intramolecular binding interaction between its epitope and the antigen binding site and thereby establish an effective concentration of the blocking moiety at the antigen binding site to facilitate the competitive binding of the blocking moiety at the antigen binding site and modulate the binding activity of the antigen binding domain.

In some embodiments, the blocking construct is crosslinked to an antigen binding domain. Whereby, upon activation of the cleavable linker by the trigger, the cleavable linker cleaves the blocking construct at the cleavable linker to dissociate the epitope of the blocking moiety from the blocking construct and thereby decrease the effective concentration of the blocking moiety at the antigen binding site to further modulate the binding activity of the antigen binding domain.

In accordance with an embodiment, a blocked immunoglobulin complex includes an immunoglobulin crosslinked to a set of one or more blocking constructs.

In accordance with an embodiment, a method of modifying the binding activity of antigen binding domain includes: providing a set of one or more blocking constructs as provided herein, and crosslinking the set of one or more blocking constructs to an antigen binding domain to thereby modify the binding activity of the antigen binding domain. In some embodiments, the method further includes exposing the set of one or more blocking constructs to an ultraviolet light trigger to activate the cleavable linker of the blocking construct to disassociate the blocking moiety from the antigen binding domain and reduce the effective concentration of block moiety at the antigen binding domain to thereby modify the binding activity antigen binding domain to an antigen.

In some embodiments, at least one crosslinker kappa light chain-binding domain in the set of one or more crosslinker kappa light chain-binding domains includes an amino acid sequence selected from the Protein L amino acid sequence set forth in any of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, and SEQ ID NO: 11, in which the amino acid residue corresponding to position 33 as set forth in the selected amino acid sequence is substituted by a photo-reactive crosslinker residue having an activation wavelength.

In some embodiments, at least one crosslinker kappa light chain-binding domain includes the Protein L amino acid sequence set forth in SEQ ID NO: 1. In some embodiments, at least one crosslinker kappa light chain-binding domain includes the Protein L amino acid sequence set forth in SEQ ID NO: 2. In some embodiments, at least one crosslinker kappa light chain-binding domain includes the Protein L amino acid sequence set forth in SEQ ID NO: 3. In some embodiments, at least one crosslinker kappa light chain-binding domain includes the Protein L amino acid sequence set forth in SEQ ID NO: 4. In some embodiments, at least one crosslinker kappa light chain-binding domain includes the Protein L amino acid sequence set forth in SEQ ID NO: 5. In some embodiments, at least one crosslinker kappa light chain-binding domain includes the Protein L amino acid sequence set forth in SEQ ID NO: 6. In some embodiments, at least one crosslinker kappa light chain-binding domain includes the Protein L amino acid sequence set forth in SEQ ID NO: 7. In some embodiments, at least one crosslinker kappa light chain-binding domain includes the Protein L amino acid sequence set forth in SEQ ID NO: 8. In some embodiments, at least one crosslinker kappa light chain-binding domain includes the Protein L amino acid sequence set forth in SEQ ID NO: 9. In some embodiments, at least one crosslinker kappa light chain-binding domain includes the Protein L amino acid sequence set forth in SEQ ID NO: 10. In some embodiments, at least one crosslinker kappa light chain-binding domain includes the Protein L amino acid sequence set forth in SEQ ID NO: 11.

In some embodiments, the photo-reactive crosslinker residue may be selected from a 4-benzoyl-L-phenylalanine (BpA) residue, a (2R)-2-amino-3-fluoro-3-(4-((2-nitrobenzyl)oxy) phenyl) propanoic acid residue (FnbY), a p-benzoyl-L-phenylalanine (pBpA), a n-(Fluoroacetyl)phenylalanine residue, a p-2′-fluoroacetyl-phenylalanine (Ffact) residue, a p-azidophenylalanine (pAzF), a p-vinylsulfonamido-(S)-phenylalanine residue, and a p-isothiocyanate phenylalanine (pNCSF) residue.

In some embodiments, the photo-reactive crosslinker residue is a 4-benzoyl-L-phenylalanine (BpA) residue. In some embodiments, the photo-reactive crosslinker residue is a (2R)-2-amino-3-fluoro-3-(4-((2-nitrobenzyl)oxy) phenyl) propanoic acid residue (FnbY). In some embodiments, the photo-reactive crosslinker residue is a p-benzoyl-L-phenylalanine (pBpA). In some embodiments, the photo-reactive crosslinker residue is a n-(Fluoroacetyl)phenylalanine residue.

In some embodiments, the photo-reactive crosslinker residue is a p-2′-fluoroacetyl-phenylalanine (Ffact) residue. In some embodiments, the photo-reactive crosslinker residue is a p-azidophenylalanine (pAzF). In some embodiments, the photo-reactive crosslinker residue is a p-vinylsulfonamido-(S)-phenylalanine residue. In some embodiments, the photo-reactive crosslinker residue is a p-isothiocyanate phenylalanine (pNCSF) residue. In some embodiments, the activation wavelength of the photo-reactive crosslinker residue is 365 nm. However, it will be understood by those of skill in the art that different crosslinkers exist which (and can be designed to) use different wavelengths of light for activation and crosslinking. Though several described herein use UV light for crosslinking, including 365 nm light, it will be understood that a wide range of different crosslinkers and activation wavelengths can readily be used in the methods and systems described herein.

In some embodiments, at least one crosslinker kappa light chain-binding domain in the set of one or more crosslinker kappa light chain-binding domains includes a Protein L amino acid sequence selected from the Protein L amino acid sequence set forth in any of: SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, and SEQ ID NO: 22.

In some embodiments, at least one crosslinker kappa light chain-binding domain includes the Protein L amino acid sequence set forth in SEQ ID NO: 12. In some embodiments, at least one crosslinker kappa light chain-binding domain includes the Protein L amino acid sequence set forth in SEQ ID NO: 13. In some embodiments, at least one crosslinker kappa light chain-binding domain includes the Protein L amino acid sequence set forth in SEQ ID NO: 14. In some embodiments, at least one crosslinker kappa light chain-binding domain includes the Protein L amino acid sequence set forth in SEQ ID NO: 15. In some embodiments, at least one crosslinker kappa light chain-binding domain includes the Protein L amino acid sequence set forth in SEQ ID NO: 16. In some embodiments, at least one crosslinker kappa light chain-binding domain includes the Protein L amino acid sequence set forth in SEQ ID NO: 17. In some embodiments, at least one crosslinker kappa light chain-binding domain includes the Protein L amino acid sequence set forth in SEQ ID NO: 18. In some embodiments, least one crosslinker kappa light chain-binding domain includes the Protein L amino acid sequence set forth in SEQ ID NO: 19. In some embodiments, at least one crosslinker kappa light chain-binding domain includes the Protein L amino acid sequence set forth in SEQ ID NO: 20. In some embodiments, at least one crosslinker kappa light chain-binding domain includes the Protein L amino acid sequence set forth in SEQ ID NO: 21. In some embodiments, at least one crosslinker kappa light chain-binding domain includes the Protein L amino acid sequence set forth in SEQ ID NO: 22.

In accordance with an embodiment, at least one crosslinker kappa light chain-binding domain in the set of one or more crosslinker kappa light chain-binding domains includes a polypeptide structure represented from N-terminus to C-terminus by the formula: β₁-L₁-β₂-α-L₂-β₃-L₃-β₄. In some embodiments, the β₁ is a first beta-sheet motif including a Protein L amino acid sequence selected from the Protein L amino acid sequence corresponding to positions 1 to 9 as set forth in any of: SEQ ID NO: 1; SEQ ID NO: 1, in which the amino acid residue corresponding to position 6 as set forth in SEQ ID NO: 1 is substituted by alanine; and SEQ ID NO: 1, in which the amino acid residues corresponding to positions 1, 6, 8, and 9 as set forth in SEQ ID NO: 1 are substituted by, respectively, glutamine, glutamate, isoleucine, and tyrosine.

In some embodiments, the β₂ is a second beta-sheet motif including a Protein L amino acid sequence selected from the Protein L amino acid sequence corresponding to positions 15 to 23 as set forth in any of: SEQ ID NO: 1; SEQ ID NO: 1, in which the amino acid residue corresponding to position 15 as set forth in SEQ ID NO: 1 is substituted by threonine; SEQ ID NO: 1, in which the amino acid residues corresponding to positions 15 and 17 as set forth in SEQ ID NO: 1 are substituted by, respectively, threonine and asparagine; and SEQ ID NO: 1, in which the amino acid residues corresponding to positions 15 and 19 as set forth in SEQ ID NO: 1 are substituted by, respectively, valine and threonine.

In some embodiments, the β₃ is a third beta-sheet motif including a Protein L amino acid sequence selected from the Protein L amino acid sequence corresponding to positions 45 to 50 as set forth in any of: SEQ ID NO: 1; and SEQ ID NO: 1, in which the amino acid residues corresponding to positions 47, 49, and 50 as set forth in SEQ ID NO: 1 are substituted by, respectively, valine, valine, and alanine.

In some embodiments, the β₄ is a fourth beta-sheet motif including a Protein L amino acid sequence selected from the Protein L amino acid sequence corresponding to positions 55 to 61 as set forth in any of: SEQ ID NO: 1; SEQ ID NO: 1, in which the amino acid residues corresponding to positions 55 and 56 as set forth in SEQ ID NO: 1 are substituted by, respectively, threonine and leucine; SEQ ID NO: 1, in which the amino acid residues corresponding to positions 55 and 56 as set forth in SEQ ID NO: 1 are substituted by, respectively, threonine and isoleucine; and SEQ ID NO: 1, in which the amino acid residues corresponding to positions 55, 56, and 59 as set forth in SEQ ID NO: 1 are substituted by, respectively, threonine, isoleucine, and arginine.

In some embodiments, the α is an alpha helix motif including a Protein L amino acid sequence selected from the Protein L amino acid sequence corresponding to positions 24 to 39 as set forth in any of: SEQ ID NO: 1; SEQ ID NO: 1, in which the amino acid residue corresponding to position 25 as set forth in SEQ ID NO: 1 is substituted by alanine; SEQ ID NO: 1, in which the amino acid residues corresponding to positions 25 and 39 as set forth in SEQ ID NO: 1 are substituted by, respectively, alanine and serine; SEQ ID NO: 1, in which the amino acid residues corresponding to positions 25, 26, 28, 29, 30, 37, and 39 as set forth in SEQ ID NO: 1 are substituted by, respectively, alanine, lysine, valine, serine, aspartate, alanine, and lysine; SEQ ID NO: 1, in which the amino acid residues corresponding to positions 26, 29, 37, and 39 as set forth in SEQ ID NO: 1 are substituted by, respectively, lysine, serine, threonine, and lysine; SEQ ID NO: 1, in which the amino acid residue corresponding to position 30 as set forth in SEQ ID NO: 1 is substituted by lysine; SEQ ID NO: 1, in which the amino acid residues corresponding to positions 30 and 36 as set forth in SEQ ID NO: 1 are substituted by, respectively, lysine and asparagine; SEQ ID NO: 1, in which the amino acid residue corresponding to position 33 as set forth in the selected amino acid sequence is substituted by a photo-reactive crosslinker residue having an activation wavelength, the photo-reactive crosslinker residue; and SEQ ID NO: 1, in which the amino acid residues corresponding to positions 37 and 39 as set forth in SEQ ID NO: 1 are substituted by, respectively, alanine and lysine.

In some embodiments, the L₁ is an amino acid linker including a Protein L amino acid sequence selected from the Protein L amino acid sequence corresponding to positions 10 to 14 as set forth in any of: SEQ ID NO: 1; SEQ ID NO: 1, in which the amino acid residue corresponding to position 10 as set forth in SEQ ID NO: 1 is substituted by tyrosine; SEQ ID NO: 1, in which the amino acid residue corresponding to position 11 as set forth in SEQ ID NO: 1 is substituted by glutamate; SEQ ID NO: 1, in which the amino acid residue corresponding to position 12 as set forth in SEQ ID NO: 1 is substituted by asparagine; and SEQ ID NO: 1, in which the amino acid residue corresponding to position 13 as set forth in SEQ ID NO: 1 is substituted by serine.

In some embodiments the L₂ is an amino acid linker including a Protein L amino acid sequence selected from the Protein L amino acid sequence corresponding to positions 40 to 44 as set forth in any of: SEQ ID NO: 1; SEQ ID NO: 1, in which the amino acid residue corresponding to position 41 as set forth in SEQ ID NO: 1 is substituted by aspartate; SEQ ID NO: 1, in which the amino acid residue corresponding to position 41 as set forth in SEQ ID NO: 1 is substituted by glutamate; SEQ ID NO: 1, in which the amino acid residues corresponding to positions 41 and 44 as set forth in SEQ ID NO: 1 are substituted by, respectively, glutamate and lysine; and SEQ ID NO: 1, in which the amino acid residues corresponding to positions 41, 42, and 44 as set forth in SEQ ID NO: 1 are substituted by, respectively, glutamate, histidine, and lysine.

In some embodiments, the L₃ is an amino acid linker including a Protein L amino acid sequence selected from the Protein L amino acid sequence corresponding to positions 51 to 54 as set forth in any of: SEQ ID NO: 1; and SEQ ID NO: 1, in which the amino acid residue corresponding to position 52 as set forth in SEQ ID NO: 1 is substituted by lysine.

In some embodiments, the 1 is a first beta-sheet motif including a Protein L amino acid sequence selected from the Protein L amino acid sequence corresponding to positions 1 to 9 as set forth in SEQ ID NO 1.

In some embodiments, the β₁ is a first beta-sheet motif including a Protein L amino acid sequence selected from the Protein L amino acid sequence corresponding to positions 1 to 9 as set forth in SEQ ID NO 1, in which the amino acid residue corresponding to position 6 as set forth in SEQ ID NO: 1 is substituted by alanine.

In some embodiments, the β₁ is a first beta-sheet motif including a Protein L amino acid sequence selected from the Protein L amino acid sequence corresponding to positions 1 to 9 as set forth in SEQ ID NO 1, in which the amino acid residues corresponding to positions 1, 6, 8, and 9 as set forth in SEQ ID NO: 1 are substituted by, respectively, glutamine, glutamate, isoleucine, and tyrosine.

In some embodiments, the β₂ is a second beta-sheet motif including a Protein L amino acid sequence selected from the Protein L amino acid sequence corresponding to positions 15 to 23 as set forth in SEQ ID NO: 1.

In some embodiments, the β₂ is a second beta-sheet motif including a Protein L amino acid sequence selected from the Protein L amino acid sequence corresponding to positions 15 to 23 set forth in SEQ ID NO: 1, in which the amino acid residue corresponding to position 15 as set forth in SEQ ID NO: 1 is substituted by threonine.

In some embodiments, the β₂ is a second beta-sheet motif including a Protein L amino acid sequence selected from the Protein L amino acid sequence corresponding to positions 15 to 23 set forth in SEQ ID NO: 1, in which the amino acid residues corresponding to positions 15 and 17 as set forth in SEQ ID NO: 1 are substituted by, respectively, threonine and asparagine

In some embodiments, the β₂ is a second beta-sheet motif including a Protein L amino acid sequence selected from the Protein L amino acid sequence corresponding to positions 15 to 23 set forth in SEQ ID NO: 1, in which the amino acid residues corresponding to positions 15 and 19 as set forth in SEQ ID NO: 1 are substituted by, respectively, valine and threonine.

In some embodiments, the β₃ is a third beta-sheet motif including a Protein L amino acid sequence selected from the Protein L amino acid sequence corresponding to positions 45 to 50 as set forth in set forth in SEQ ID NO: 1

In some embodiments, the β₃ is a third beta-sheet motif including a Protein L amino acid sequence selected from the Protein L amino acid sequence corresponding to positions 45 to 50 as set forth in set forth in SEQ ID NO: 1, in which the amino acid residues corresponding to positions 47, 49, and 50 as set forth in SEQ ID NO: 1 are substituted by, respectively, valine, valine, and alanine.

In some embodiments, the β₄ is a fourth beta-sheet motif including a Protein L amino acid sequence selected from the Protein L amino acid sequence corresponding to positions 55 to 61 as set forth in SEQ ID NO: 1.

In some embodiments, the β₄ is a fourth beta-sheet motif including a Protein L amino acid sequence selected from the Protein L amino acid sequence corresponding to positions 55 to 61 as set forth in SEQ ID NO: 1, in which the amino acid residues corresponding to positions 55 and 56 as set forth in SEQ ID NO: 1 are substituted by, respectively, threonine and leucine.

In some embodiments, the β₄ is a fourth beta-sheet motif including a Protein L amino acid sequence selected from the Protein L amino acid sequence corresponding to positions 55 to 61 as set forth in SEQ ID NO: 1, in which the amino acid residues corresponding to positions 55 and 56 as set forth in SEQ ID NO: 1 are substituted by, respectively, threonine and isoleucine.

In some embodiments, the β₄ is a fourth beta-sheet motif including a Protein L amino acid sequence selected from the Protein L amino acid sequence corresponding to positions 55 to 61 as set forth in SEQ ID NO: 1, in which the amino acid residues corresponding to positions 55, 56, and 59 as set forth in SEQ ID NO: 1 are substituted by, respectively, threonine, isoleucine, and arginine.

In some embodiments, the α is an alpha helix motif including a Protein L amino acid sequence selected from the Protein L amino acid sequence corresponding to positions 24 to 39 as set forth in SEQ ID NO: 1.

In some embodiments, the α is an alpha helix motif including a Protein L amino acid sequence selected from the Protein L amino acid sequence corresponding to positions 24 to 39 as set forth in SEQ ID NO: 1, in which the amino acid residue corresponding to position 25 as set forth in SEQ ID NO: 1 is substituted by alanine.

In some embodiments, the α is an alpha helix motif including a Protein L amino acid sequence selected from the Protein L amino acid sequence corresponding to positions 24 to 39 as set forth in SEQ ID NO: 1, in which the amino acid residues corresponding to positions 25 and 39 as set forth in SEQ ID NO: 1 are substituted by, respectively, alanine and serine.

In some embodiments, the α is an alpha helix motif including a Protein L amino acid sequence selected from the Protein L amino acid sequence corresponding to positions 24 to 39 as set forth in SEQ ID NO: 1, in which the amino acid residues corresponding to positions 25, 26, 28, 29, 30, 37, and 39 as set forth in SEQ ID NO: 1 are substituted by, respectively, alanine, lysine, valine, serine, aspartate, alanine, and lysine.

In some embodiments, the α is an alpha helix motif including a Protein L amino acid sequence selected from the Protein L amino acid sequence corresponding to positions 24 to 39 as set forth in SEQ ID NO: 1, in which the amino acid residues corresponding to positions 26, 29, 37, and 39 as set forth in SEQ ID NO: 1 are substituted by, respectively, lysine, serine, threonine, and lysine.

In some embodiments, the α is an alpha helix motif including a Protein L amino acid sequence selected from the Protein L amino acid sequence corresponding to positions 24 to 39 as set forth in SEQ ID NO: 1, in which the amino acid residue corresponding to position 30 as set forth in SEQ ID NO: 1 is substituted by lysine.

In some embodiments, the α is an alpha helix motif including a Protein L amino acid sequence selected from the Protein L amino acid sequence corresponding to positions 24 to 39 as set forth in SEQ ID NO: 1, in which the amino acid residues corresponding to positions 30 and 36 as set forth in SEQ ID NO: 1 are substituted by, respectively, lysine and asparagine.

In some embodiments, the α is an alpha helix motif including a Protein L amino acid sequence selected from the Protein L amino acid sequence corresponding to positions 24 to 39 as set forth in SEQ ID NO: 1, in which the amino acid residues corresponding to positions 30 and 36 as set forth in SEQ ID NO: 1 are substituted by, respectively, lysine and asparagine.

In some embodiments, the α is an alpha helix motif including a Protein L amino acid sequence selected from the Protein L amino acid sequence corresponding to positions 24 to 39 as set forth in SEQ ID NO: 1, in which the amino acid residue corresponding to position 33 as set forth in the selected amino acid sequence is substituted by a photo-reactive crosslinker residue having an activation wavelength.

In some embodiments, the α is an alpha helix motif including a Protein L amino acid sequence selected from the Protein L amino acid sequence corresponding to positions 24 to 39 as set forth in SEQ ID NO: 1, in which the amino acid residues corresponding to positions 37 and 39 as set forth in SEQ ID NO: 1 are substituted by, respectively, alanine and lysine.

In some embodiments, the L₁ is an amino acid linker including a Protein L amino acid sequence selected from the Protein L amino acid sequence corresponding to positions 10 to 14 as set forth in SEQ ID NO: 1.

In some embodiments, the L₁ is an amino acid linker including a Protein L amino acid sequence selected from the Protein L amino acid sequence corresponding to positions 10 to 14 as set forth in SEQ ID NO: 1, in which the amino acid residue corresponding to position 10 as set forth in SEQ ID NO: 1 is substituted by tyrosine.

In some embodiments, the L₁ is an amino acid linker including a Protein L amino acid sequence selected from the Protein L amino acid sequence corresponding to positions 10 to 14 as set forth in SEQ ID NO: 1, in which the amino acid residue corresponding to position 11 as set forth in SEQ ID NO: 1 is substituted by glutamate.

In some embodiments, the L₁ is an amino acid linker including a Protein L amino acid sequence selected from the Protein L amino acid sequence corresponding to positions 10 to 14 as set forth in SEQ ID NO: 1, in which the amino acid residue corresponding to position 12 as set forth in SEQ ID NO: 1 is substituted by asparagine.

In some embodiments, the L₁ is an amino acid linker including a Protein L amino acid sequence selected from the Protein L amino acid sequence corresponding to positions 10 to 14 as set forth in SEQ ID NO: 1, in which the amino acid residue corresponding to position 13 as set forth in SEQ ID NO: 1 is substituted by serine.

In some embodiments, the L₂ is an amino acid linker including a Protein L amino acid sequence selected from the Protein L amino acid sequence corresponding to positions 40 to 44 as set forth in SEQ ID NO: 1.

In some embodiments, the L₂ is an amino acid linker including a Protein L amino acid sequence selected from the Protein L amino acid sequence corresponding to positions 40 to 44 as set forth in SEQ ID NO: 1, in which the amino acid residue corresponding to position 41 as set forth in SEQ ID NO: 1 is substituted by aspartate.

In some embodiments, the L₂ is an amino acid linker including a Protein L amino acid sequence selected from the Protein L amino acid sequence corresponding to positions 40 to 44 as set forth in SEQ ID NO: 1, in which the amino acid residue corresponding to position 41 as set forth in SEQ ID NO: 1 is substituted by glutamate.

In some embodiments, the L₂ is an amino acid linker including a Protein L amino acid sequence selected from the Protein L amino acid sequence corresponding to positions 40 to 44 as set forth in SEQ ID NO: 1, in which the amino acid residues corresponding to positions 41 and 44 as set forth in SEQ ID NO: 1 are substituted by, respectively, glutamate and lysine.

In some embodiments, the L₂ is an amino acid linker including a Protein L amino acid sequence selected from the Protein L amino acid sequence corresponding to positions 40 to 44 as set forth in SEQ ID NO: 1, in which the amino acid residues corresponding to positions 41 and 44 as set forth in SEQ ID NO: 1 are substituted by, respectively, glutamate and lysine.

In some embodiments, the L₃ is an amino acid linker including a Protein L amino acid sequence selected from the Protein L amino acid sequence corresponding to positions 51 to 54 as set forth in SEQ ID NO: 1.

In some embodiments, the L₃ is an amino acid linker including a Protein L amino acid sequence selected from the Protein L amino acid sequence corresponding to positions 51 to 54 as set forth in SEQ ID NO: 1.

In some embodiments, the L₃ is an amino acid linker including a Protein L amino acid sequence selected from the Protein L amino acid sequence corresponding to positions 51 to 54 as set forth in SEQ ID NO: 1, in which the amino acid residue corresponding to position 52 as set forth in SEQ ID NO: 1 is substituted by lysine.

In some embodiments, the L₃ is an amino acid linker including a Protein L amino acid sequence selected from the Protein L amino acid sequence corresponding to positions 51 to 54 as set forth in SEQ ID NO: 1, in which the amino acid residue corresponding to position 52 as set forth in SEQ ID NO: 1 is substituted by lysine.

In accordance with an embodiment, at least one crosslinker kappa light chain-binding domain in a set of one or more crosslinker kappa light chain-binding domains includes an engineered Protein L kappa light chain-binding domain including a crosslinker alpha helix motif having a structure represented from N-terminus to C-terminus by the amino acid sequence set forth in any of: Phe-Glu-Glu-Ala-Thr-Ala-Glu-Ala-Tyr-X-Tyr-Ala-Asp-Leu-Leu-Ala (SEQ ID NO: 23); Phe-Ala-Lys-Ala-Val-Ser-Asp-Ala-Tyr-X-Tyr-Ala-Asp-Ala-Leu-Lys (SEQ ID NO: 24); Phe-Glu-Glu-Ala-Thr-Ala-Lys-Ala-Tyr-X-Tyr-Ala-Asp-Leu-Leu-Ala (SEQ ID NO: 25); Phe-Glu-Glu-Ala-Thr-Ala-Lys-Ala-Tyr-X-Tyr-Ala-Asn-Leu-Leu-Ala (SEQ ID NO: 26); Phe-Glu-Lys-Ala-Thr-Ser-Glu-Ala-Tyr-X-Tyr-Ala-Asp-Thr-Leu-Lys (SEQ ID NO: 27); Phe-Glu-Glu-Ala-Thr-Ala-Glu-Ala-Tyr-X-Tyr-Ala-Asp-Ala-Leu-Lys (SEQ ID NO: 28); Phe-Ala-Glu-Ala-Thr-Ala-Glu-Ala-Tyr-X-Tyr-Ala-Asp-Leu-Leu-Ala (SEQ ID NO: 29); and Phe-Ala-Glu-Ala-Thr-Ala-Glu-Ala-Tyr-X-Tyr-Ala-Asp-Leu-Leu-Ser (SEQ ID NO: 30), and in which the amino acid residue corresponding to position X is substituted by the photo-reactive crosslinker residue.

In some embodiments, at least one crosslinker kappa light chain-binding domain in a set of one or more crosslinker kappa light chain-binding domains includes an engineered Protein L kappa light chain-binding domain including a crosslinker alpha helix motif having an amino acid sequence represented from N-terminus to C-terminus by the formula: Phe-Glu-Glu-Ala-Thr-Ala-Glu-Ala-Tyr-X-Tyr-Ala-Asp-Leu-Leu-Ala (SEQ ID NO: 23), and in which the amino acid residue corresponding to position X is substituted by the photo-reactive crosslinker residue.

In some embodiments, at least one crosslinker kappa light chain-binding domain in a set of one or more crosslinker kappa light chain-binding domains includes an engineered Protein L kappa light chain-binding domain including a crosslinker alpha helix motif having an amino acid sequence represented from N-terminus to C-terminus by the formula: Phe-Ala-Lys-Ala-Val-Ser-Asp-Ala-Tyr-X-Tyr-Ala-Asp-Ala-Leu-Lys (SEQ ID NO: 24), and in which the amino acid residue corresponding to position X is substituted by the photo-reactive crosslinker residue.

In some embodiments, at least one crosslinker kappa light chain-binding domain in a set of one or more crosslinker kappa light chain-binding domains includes an engineered Protein L kappa light chain-binding domain including a crosslinker alpha helix motif having an amino acid sequence represented from N-terminus to C-terminus by the formula: Phe-Glu-Glu-Ala-Thr-Ala-Lys-Ala-Tyr-X-Tyr-Ala-Asp-Leu-Leu-Ala (SEQ ID NO: 25), and in which the amino acid residue corresponding to position X is substituted by the photo-reactive crosslinker residue.

In some embodiments, at least one crosslinker kappa light chain-binding domain in a set of one or more crosslinker kappa light chain-binding domains includes an engineered Protein L kappa light chain-binding domain including a crosslinker alpha helix motif having an amino acid sequence represented from N-terminus to C-terminus by the formula: Phe-Glu-Glu-Ala-Thr-Ala-Lys-Ala-Tyr-X-Tyr-Ala-Asn-Leu-Leu-Ala (SEQ ID NO: 26), and in which the amino acid residue corresponding to position X is substituted by the photo-reactive crosslinker residue.

In some embodiments, at least one crosslinker kappa light chain-binding domain in a set of one or more crosslinker kappa light chain-binding domains includes an engineered Protein L kappa light chain-binding domain including a crosslinker alpha helix motif having an amino acid sequence represented from N-terminus to C-terminus by the formula: Phe-Glu-Lys-Ala-Thr-Ser-Glu-Ala-Tyr-X-Tyr-Ala-Asp-Thr-Leu-Lys (SEQ ID NO: 27), and in which the amino acid residue corresponding to position X is substituted by the photo-reactive crosslinker residue.

In some embodiments, at least one crosslinker kappa light chain-binding domain in a set of one or more crosslinker kappa light chain-binding domains includes an engineered Protein L kappa light chain-binding domain including a crosslinker alpha helix motif having an amino acid sequence represented from N-terminus to C-terminus by the formula: Phe-Glu-Glu-Ala-Thr-Ala-Glu-Ala-Tyr-X-Tyr-Ala-Asp-Ala-Leu-Lys (SEQ ID NO: 28), and in which the amino acid residue corresponding to position X is substituted by the photo-reactive crosslinker residue.

In some embodiments, at least one crosslinker kappa light chain-binding domain in a set of one or more crosslinker kappa light chain-binding domains includes an engineered Protein L kappa light chain-binding domain including a crosslinker alpha helix motif having an amino acid sequence represented from N-terminus to C-terminus by the formula: Phe-Ala-Glu-Ala-Thr-Ala-Glu-Ala-Tyr-X-Tyr-Ala-Asp-Leu-Leu-Ala (SEQ ID NO: 29), and in which the amino acid residue corresponding to position X is substituted by the photo-reactive crosslinker residue.

In some embodiments, at least one crosslinker kappa light chain-binding domain in the set of one or more crosslinker kappa light chain-binding domains includes an engineered Protein L kappa light chain-binding domain including a crosslinker alpha helix motif having an amino acid sequence represented from N-terminus to C-terminus by the formula: Phe-Ala-Glu-Ala-Thr-Ala-Glu-Ala-Tyr-X-Tyr-Ala-Asp-Leu-Leu-Ser (SEQ ID NO: 30), and in which the amino acid residue corresponding to position X is substituted by the photo-reactive crosslinker residue.

In some embodiments, at least one crosslinker kappa light chain-binding domain in a set of one or more crosslinker kappa light chain-binding domains includes an engineered Protein L kappa light chain-binding domain selected from a domain C*, a domain C1, a domain C2, a domain C3, a domain C4, a domain B1, a domain B2, a domain B3, a domain B4, and a domain B5, in which the selected Protein L kappa light chain-binding domain includes a photo-reactive crosslinker residue having an activation wavelength.

In some embodiments, the engineered Protein L kappa light chain-binding domain is a domain C*. In some embodiments, the engineered Protein L kappa light chain-binding domain is a domain C1. In some embodiments, the engineered Protein L kappa light chain-binding domain is a domain C2. In some embodiments, the engineered Protein L kappa light chain-binding domain is a domain C3. In some embodiments, the engineered Protein L kappa light chain-binding domain is a domain C4. In some embodiments, the engineered Protein L kappa light chain-binding domain is a domain B1. In some embodiments, the engineered Protein L kappa light chain-binding domain is a domain B2. In some embodiments, the engineered Protein L kappa light chain-binding domain is, a domain B3. In some embodiments, the engineered Protein L kappa light chain-binding domain is a domain B4. In some embodiments, the engineered Protein L kappa light chain-binding domain is a domain B5.

In some embodiments, the photo-reactive crosslinker residue is selected from a 4-benzoyl-L-phenylalanine (BpA) residue, a (2R)-2-amino-3-fluoro-3-(4-((2-nitrobenzyl)oxy) phenyl) propanoic acid residue (FnbY), a p-benzoyl-L-phenylalanine (pBpA), a n-(Fluoroacetyl)phenylalanine residue, a p-2′-fluoroacetyl-phenylalanine (Ffact) residue, a p-azidophenylalanine (pAzF), a p-vinylsulfonamido-(S)-phenylalanine residue, and a p-isothiocyanate phenylalanine (pNCSF) residue.

In some embodiments, the photo-reactive crosslinker residue is a 4-benzoyl-L-phenylalanine (BpA) residue. In some embodiments, the photo-reactive crosslinker residue is a (2R)-2-amino-3-fluoro-3-(4-((2-nitrobenzyl)oxy) phenyl) propanoic acid residue (FnbY). In some embodiments, the photo-reactive crosslinker residue is a p-benzoyl-L-phenylalanine (pBpA). In some embodiments, the photo-reactive crosslinker residue is a n-(Fluoroacetyl)phenylalanine residue. In some embodiments, the photo-reactive crosslinker residue is a p-2′-fluoroacetyl-phenylalanine (Ffact) residue. In some embodiments, the photo-reactive crosslinker residue is a p-azidophenylalanine (pAzF). In some embodiments, the photo-reactive crosslinker residue is a p-vinylsulfonamido-(S)-phenylalanine residue. In some embodiments, the photo-reactive crosslinker residue is a p-isothiocyanate phenylalanine (pNCSF) residue. In some embodiments, the activation wavelength of the photo-reactive crosslinker residue is 365 nm.

In accordance with an embodiment, a blocking construct for modulating the binding activity of an antigen binding domain includes a kappa light chain-binding polypeptide operatively connected to a blocking moiety via a flexible tether to form a blocking construct. In some embodiments, the kappa light chain-binding polypeptide is configured to have, when in the proximity of a kappa light chain of an antigen binding domain, a binding interaction with the kappa light chain and thereby form a non-covalent bond between the blocking construct and the antigen binding domain, and whereby, upon exposure of a photo-reactive crosslinker residue of a kappa light chain binding domain forming the non-covalent bond to light of the activation wavelength of the photo-reactive crosslinker residue, activate the photo-reactive crosslinker residue and crosslink the kappa light chain binding domain forming the non-covalent bond to the kappa light chain and thereby form a covalent bond between the blocking construct and antigen binding domain. In some embodiments, the blocking moiety includes an epitope configured to competitively bind to an antigen binding site of the antigen binding domain; and the flexible tether includes a flexible linker, the flexible linker operatively connected at a proximal end to the kappa light chain-binding polypeptide and at a distal end to the blocking moiety and configured to have an end-to-end length to tether the blocking moiety at a sufficient movement radius for the blocking moiety to have an intramolecular binding interaction between its epitope and the antigen binding site and establish an effective concentration of the blocking moiety at the antigen binding site and thereby facilitate the competitive binding of the blocking moiety at the antigen binding site and modulate the binding activity of the antigen binding domain.

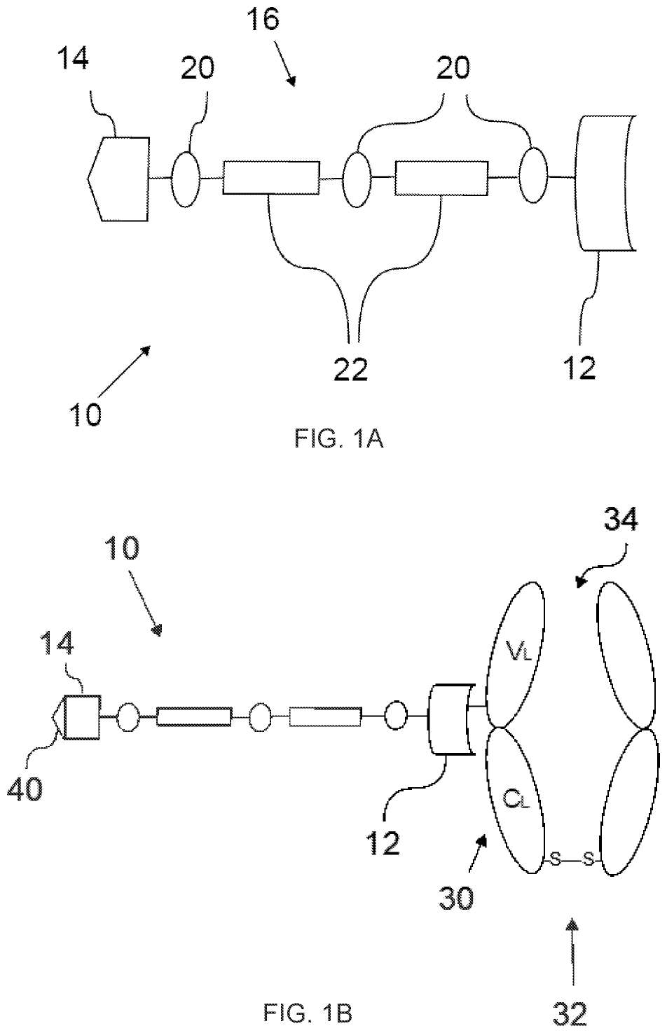

FIG. 1A shows a graphical representation of a blocking construct 10 including a kappa light chain-binding domain 12 operatively connected to a blocking moiety 14 by a flexible tether 16 including tri-glycine motifs 20 and alpha helix motifs 22.

FIG. 1B shows a kappa light chain-binding domain 12 crosslinked to a kappa light chain 30 of an antigen binding domain 32. As shown in FIG. 1B, a blocking moiety 14 includes an epitope 40 configured to competitively bind to an antigen binding site 34 of antigen binding domain 32.

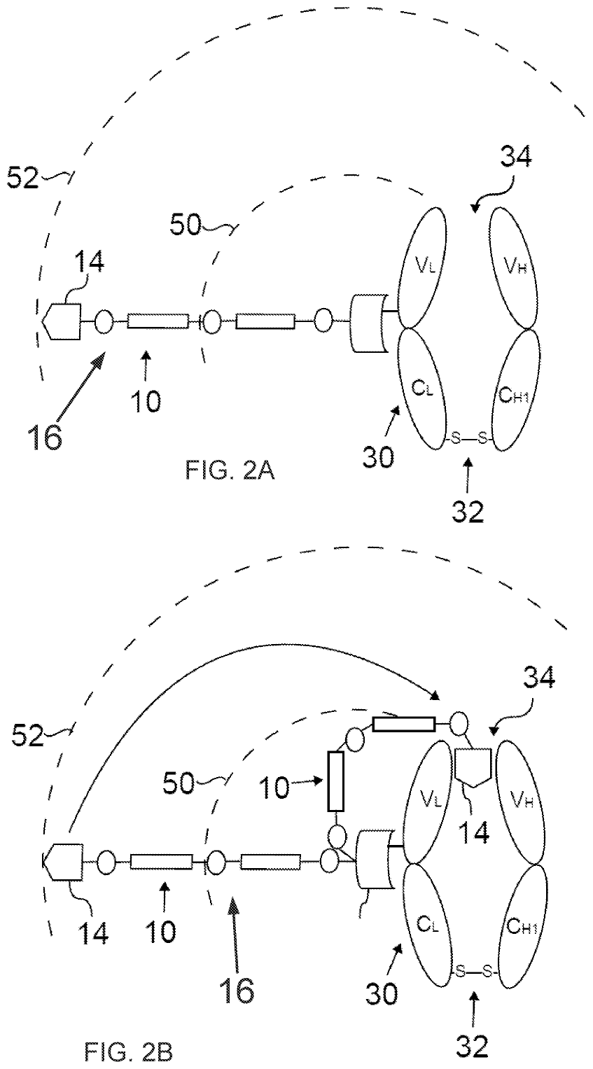

FIGS. 2A and 2B show graphical representations of a blocking construct 10 crosslinked to an antigen binding domain 32 and including a flexible tether 16 configured to bend and have sufficient end-to-end length to present a blocking moiety to an antigen binding site of antigen binding domain 34. As shown in FIG. 2A, a blocking construct 10 has an end-to-end length that is greater than a minimum movement radius 50. As shown in FIG. 2B, flexible tether 16 allows blocking moiety 14 to have a movement radius less than a maximum movement radius 52 to thereby allow blocking moiety 14 to establish an effective concentration at antigen binding site 34 and competitively bind with antigen.

In some embodiments, the blocking construct, in which the flexible tether includes a flexible portion and a rigid portion. In some embodiments, the flexible tether includes one or more repeating motifs of the structure (X—Y)ₙ, in which X and are, respectively a flexible portion operatively connected to a rigid portion and n is the number repeats. In some embodiments, the rigid portion has a persistence length of from In some embodiments, the n equals one, two, three, four, five, six, seven, eight, nine, ten, eleven, twelve, thirteen, fourteen, fifteen, sixteen, seventeen, eighteen, nineteen, or, twenty. In some embodiments, the flexible portion is a (G₂S) flexible portion including the amino acid sequence Gly-Gly-Ser. In other embodiments, the flexible portion is a (G₃S) flexible portion including the amino acid sequence Gly-Gly-Gly-Ser. In some embodiments, the flexible portion is a (G₄S) flexible portion including the amino acid sequence Gly-Gly-Gly-Gly-Ser (e.g., SEQ ID NO: 84; also positions 1-5 of SEQ ID NO: 34 and SEQ ID NO: 35). In some embodiments, the rigid portion has a persistence length of from 1.0 angstrom (Å) to 2.0 Å, of from 2.0 Å to 3.0 Å, of from 2.0 Å to 3.0 Å, of from 3.0 Å to 4.0 Å, of from 4.0 Å to 5.0 Å, of from 6.0 Å to 7.0 Å, of from 7.0 Å to 8.0 Å, of from 8.0 Å to 9.0 Å, of from 9.0 Å to 10.0 Å, of from 10.0 Å to 11.0 Å, of from 12.0 Å to 13.0 Å, of from 13.0 Å to 14.0 Å, of from 14.0 Å to 15.0 Å, of from 16.0 Å to 17.0 Å, of from 17.0 Å to 18.0 Å, of from 18.0 Å to 19.0 Å, and of from 19.0 Å to 20.0 Å.

In some embodiments, the rigid portion is an (EA₃K)₄ rigid portion including the amino acid sequence Glu-Ala-Ala-Ala-Lys-Glu-Ala-Ala-Ala-Lys-Glu-Ala-Ala-Ala-Lys-Glu-Ala-Ala-Ala-Lys (e.g., positions 5-25 or 31-51 of SEQ ID NO: 4). In some embodiments, the end-to-end length of the flexible tether is configured to be of from 1.0 angstrom (Å) to 5.0 Å, of from 5.0 Å to 10.0 Å, of from 10.0 Å to 15.0 Å, of from 15.0 Å to 20.0 Å, of from 20.0 Å to 25.0 Å, of from 25.0 Å to 30.0 Å, of from 30.0 Å to 35.0 Å, of from 35.0 Å to 40.0 Å, of from 45.0 Å to 50.0 Å, of from 50.0 Å to 55.0 Å, of from 55.0 Å to 60.0 Å, of from 60.0 Å to 65.0 Å, of from 65.0 Å to 70.0 Å, of from 75.0 Å to 80.0 Å, of from 80.0 Å to 85.0 Å, of from 85.0 Å to 90.0 Å, of from 95.0 Å to 100.0 Å, of from 105.0 Å to 110.0 Å, of from 115.0 Å to 120.0 Å, of from 125.0 Å to 130.0 Å, of from 135.0 Å to 140.0 Å, of from 140.0 Å to 145.0 Å, and of from 145.0 Å to 150.0 Å.

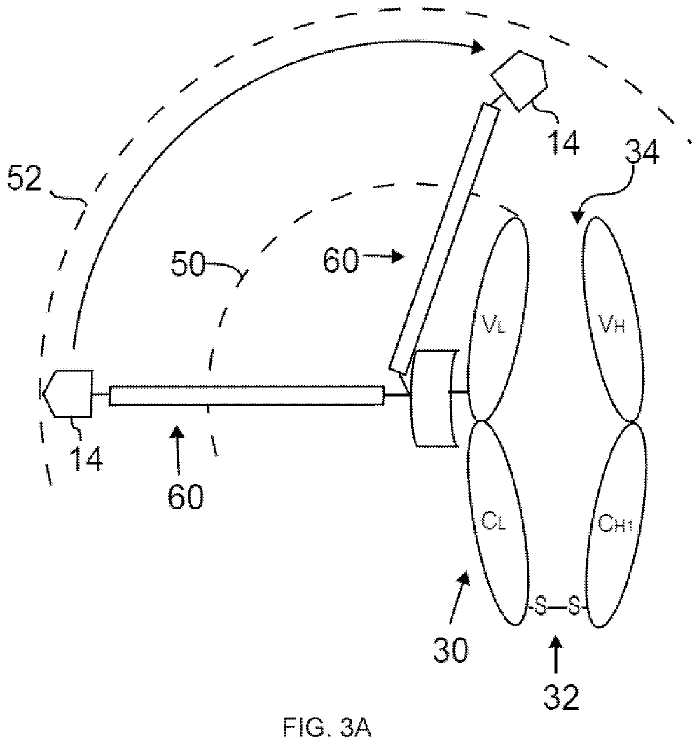

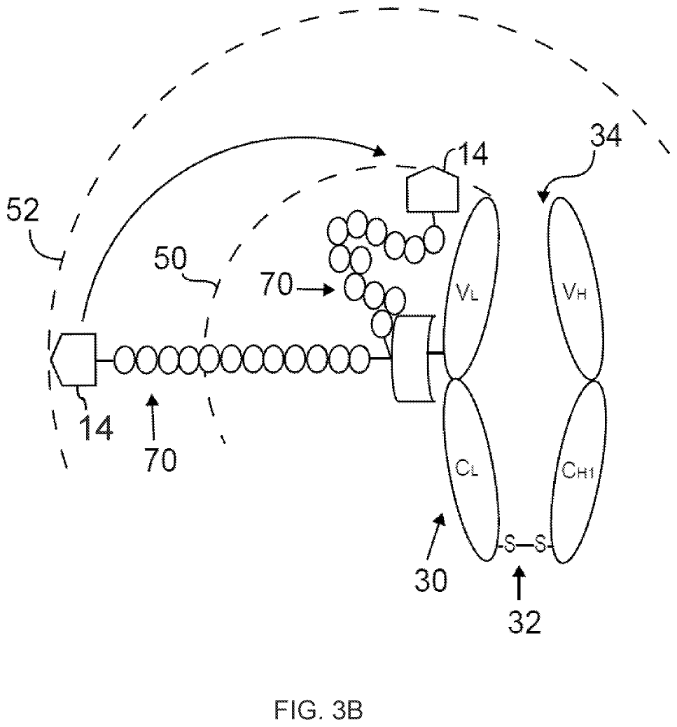

FIGS. 3A and 3B show graphical representations of a blocking constructs crosslinked to an antigen binding domain and including, respectively, a fully rigid and a fully flexible tether. As referred to herein, a fully flexible tether includes only glycine and serine and are known by skilled persons to be useful for spanning relatively short distances (e.g., <60 Å). As shown in FIG. 3A, a fully rigid tether 60 maintains a blocking moiety 14 at a maximum movement radius 52 due to steric hinderance and thus cannot present blocking moiety 14 to the antigen presenting site 34 located at minimum movement radius 50. As shown in FIG. 3B, a fully flexible tether 70 does not readily maintain blocking moiety 14 at a movement radius that is greater than minimum movement radius 50, thus lowering the effective concentration of blocking moiety 14 at antigen binding site 34. Thus, increasing the rigidity of a flexible tether to be greater than the rigidity of a fully flexible tether allows the flexible tether to effectively span distances greater that 60 Å.

Skilled persons will understand that the persistence lengths of a (GGS)₂ linker (Gly-Gly-Ser-Gly-Gly-Ser; SEQ ID NO: 83), a(S) 5 linker (S-S-S-S-S) (SEQ ID NO: 86), and a (EA₃K) (SEQ ID NO: 87) linker are, respectively, 4.5 Å, 6.2 Å, and 7.5 Å. (Rosmalen et al., Biochemistry, 56; 6565-6574, 2017). It was observed in computer simulations of Protein L binding to an antibody, that the length between the C-terminus of Protein L, bound to a kappa light chain to the far side of the binding pocket of the antibody is 60 Å, and that a curve, or bend, in the tether would be required to allow a blocking moiety to reach the binding pocket. It was observed that a fully flexible linker including only (GGS)₂ linker repeats would require 14 repeats (4.5 Å x 14) to give the tether an end-to-end length of 63 Å. However such a configuration required 84 amino acids, adding significant mass. Since a (EA₃K) linker having a persistence length of 7.5 Å from five amino acids yields more end-to-end length per amino acid than the(S) 5 linker (respectively, 1.5 Å per amino acid versus 1.0 Å) it was reasoned that a rigid portion including nine repeats of a (EA₃K) linker (7.5 Å×9=66.6 Å) could provide sufficient end-to-end length for the tether to present a blocking moiety to the binding pocket at a distance of 60 Å or greater while using less mass than the fully flexible linker configuration.

Thus, flexible tethers having an end-to-end lengths of at least 60 Å (as provided by SEQ ID NOs: 34-37) were designed to have sufficient length to present a blocking moiety to an antigen binding site and thereby establish a significant effective concentration at the binding pocket.

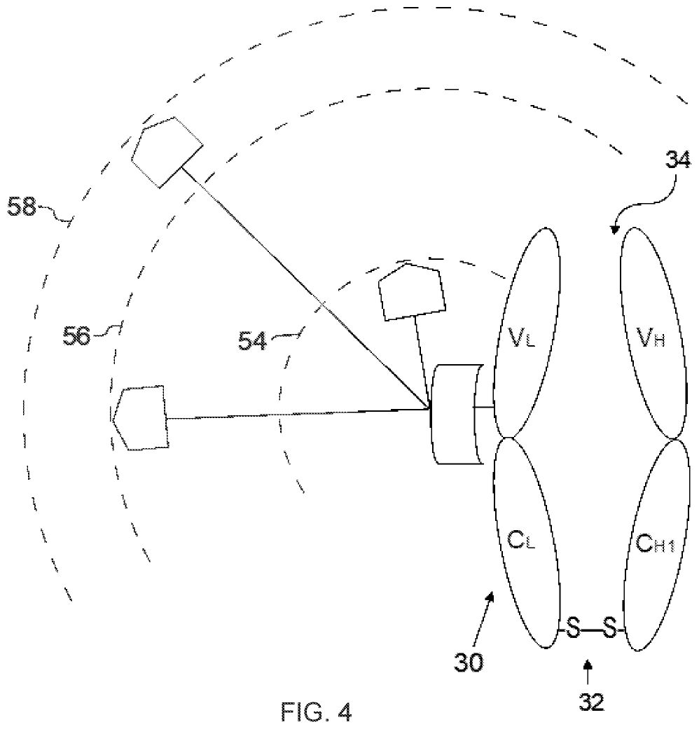

FIG. 4 shows a graphical representation of a blocking constructs crosslinked to an antigen binding domain having multiple end-to-end length radii. Skilled persons will understand that a blocking construct having an end-to-end length equal to insufficient movement radius 54 will not be useful, as it cannot present a blocking moiety to antigen binding site 34 and thus, will not establish a significant effect concentration at the antigen binding site. Conversely, blocking constructs having end-to-end lengths equal to, or greater than the movement radius of antigen binding site 34, such as movement radii 56 and 58, may reach antigen binding site 34 using a flexible tether.

In some embodiments, blocking moiety includes a polypeptide, an oligonucleotide, a glycoprotein, a fusion protein, an engineered protein, or any fragment or combination thereof.

In some embodiments, the blocking moiety further includes a cleavable linker configured to cleave upon its activation by a trigger.

In some embodiments, the blocking moiety is a polypeptide blocking moiety and the cleavable linker is a protease cleavage site configured to cleave upon its activation by a protease enzyme trigger, whereby upon activation of the protease cleavage site by the protease enzyme trigger, the protease cleavage site cleaves the blocking construct at the protease cleavage site.

In some embodiments, the blocking moiety is a polypeptide and the cleavable linker is a photo-cleavable linker having an activation wavelength, the photo-cleavable linker configured to cleave upon its activation by exposure to light of the activation wavelength, whereby upon activation of the photo-cleavable linker, the photo-cleavable linker cleaves the blocking construct at the position of photo-cleavable linker. In some embodiments, the photo-cleavable linker is a Fmoc cleavable linker. In other embodiments, the Fmoc cleavable linker is positioned at the N-terminus of the polypeptide. In further embodiment, the Fmoc cleavable linker is positioned at the C-terminus of the polypeptide.

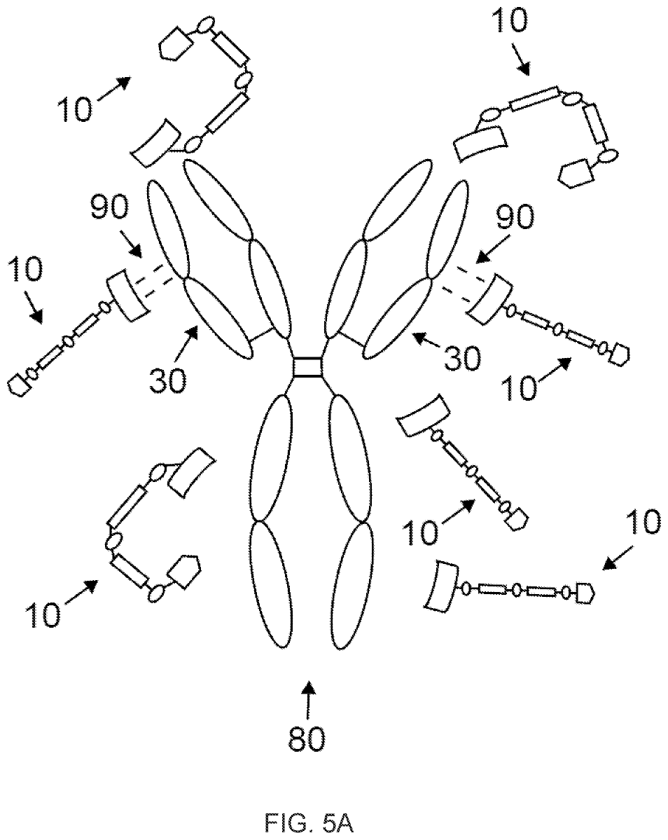

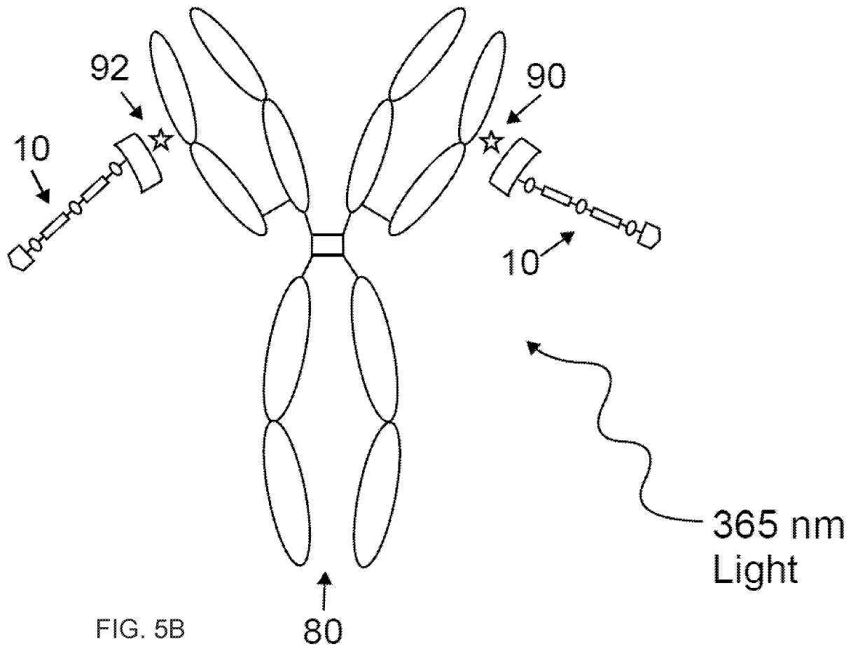

FIG. 5A shows a graphical representations of a set of one or more blocking constructs 10 including a kappa light chain-binding polypeptide that, when in the proximity of the kappa light chains of the antigen binding domains of an IgG isotype antibody, have a non-covalent binding interaction 90 with the kappa light chains to thereby form non-covalent bonds between the blocking constructs and the antigen binding domains.

FIG. 5B shows a graphical representation of blocking constructs 10 and IgG isotype antibody 80 of FIG. 5A, where exposure of 365 nm light (the activation wavelength of the photo-reactive crosslinker residues) activates the photo-reactive crosslinker residues and crosslinks the kappa light chain-binding domains to the kappa light chains and thereby forms covalent bonds 90 between blocking constructs 10 and the antigen binding domains.

In some embodiments, the blocking moiety is a polypeptide blocking moiety and the cleavable linker is a photo-cleavable linker having an activation wavelength, the photo-cleavable linker configured to cleave upon its activation by exposure to light of the activation wavelength, whereby upon activation of the photo-cleavable linker by exposure to light of the activation wavelength, the photo-cleavable linker cleaves the blocking construct at the photo-cleavable linker.

In some embodiments, the cleavable linker is a blocking moiety photo-cleavable linker having an activation wavelength. In some embodiments, the blocking moiety photo-cleavable linker is operatively connected to the N-terminus or C-terminus of the amino acid sequence set forth in SEQ ID NO: 31. In some embodiments, the blocking moiety photo-cleavable linker is operatively connected to the N-terminus or C-terminus of the amino acid sequence set forth in SEQ ID NO: 32.

In accordance with an embodiment, a blocking construct is crosslinked to an antigen binding domain, whereby, upon activation of the cleavable linker by the trigger, the cleavable linker cleaves the blocking construct at the cleavable linker to dissociate the epitope of the blocking moiety from the blocking construct and thereby decrease the effective concentration of the blocking moiety at the antigen binding site to further modulate the binding activity of the antigen binding domain.

In some embodiments, the epitope of the blocking moiety is selected from any of the group consisting of: a FLAG epitope including the amino acid sequence Asp-Tyr-Lys-Asp-Asp-Asp-Asp-Lys (SEQ ID NO: 31); and a EGFR epitope including the amino acid sequence Gln-Gly-Gln-Ser-Gly-Gln-Cys-Ile-Ser-Pro-Arg-Gly-Cys-Pro-Asp-Gly-Pro-Tyr-Val-Met-Tyr (SEQ ID NO: 32).

In some embodiments, the epitope of the blocking moiety is a FLAG epitope including the amino acid sequence Asp-Tyr-Lys-Asp-Asp-Asp-Asp-Lys (SEQ ID NO: 31).

In some embodiments, the epitope of the blocking moiety is an EGFR epitope including the amino acid sequence Gln-Gly-Gln-Ser-Gly-Gln-Cys-Ile-Ser-Pro-Arg-Gly-Cys-Pro-Asp-Gly-Pro-Tyr-Val-Met-Tyr (SEQ ID NO: 32).

In some embodiments, the kappa light chain-binding polypeptide, the blocking moiety, or the flexible tether further include a conjugation moiety selected from any of the group consisting of: a sortase recognition site including the amino acid sequence: Leu-Pro-Glu-Thr-Gly (SEQ ID NO: 33) and a click chemistry residue.

In some embodiments, the flexible tether includes a flexible linker selected from any of the group consisting of: a polypeptide flexible linker having a structure represented from N-terminus to C-terminus by the formula: (G₄S)-(EA₃K)₄-(G₄S)-(EA₃K)₄-(G₄S)-(X) (SEQ ID NO: 34), in which X is a Sortase A recognition site including the amino acid sequence Leu-Pro-Glu-Thr-Gly (SEQ ID NO: 33); a polypeptide flexible linker having a structure represented from N-terminus to C-terminus by the formula: (G₄S)-(EA₃K)₄-(G₄S)-(EA₃K)₄-(G₄S) (SEQ ID NO: 35); a polypeptide flexible linker having a structure represented from N-terminus to C-terminus by the formula: (G₂S)-(EA₃K)₄-(G₂S)-(EA₃K)₄-(G₂S)-(X) (SEQ ID NO: 36), in which X is a Sortase A recognition site including the amino acid sequence Leu-Pro-Glu-Thr-Gly (SEQ ID NO: 33); and a polypeptide flexible linker having a structure represented from N-terminus to C-terminus by the formula: (G₂S)-(EA₃K)₄-(G₂S)-(EA₃K)₄-(G₂S) (SEQ ID NO: 37).

In some embodiments, the flexible linker is a polypeptide flexible linker having a structure represented from N-terminus to C-terminus by the formula: (G₄S)-(EA₃K)₄-(G₄S)-(EA₃K)₄-(G₄S)-(X) (SEQ ID NO: 34), in which X is a Sortase A recognition site including the amino acid sequence Leu-Pro-Glu-Thr-Gly (SEQ ID NO: 33).

In some embodiments, the flexible linker is a polypeptide flexible linker having a structure represented from N-terminus to C-terminus by the formula: (G₄S)-(EA₃K)₄-(G₄S)-(EA₃K)₄-(G₄S) (SEQ ID NO: 35).

In some embodiments, the flexible linker is a polypeptide flexible linker having a structure represented from N-terminus to C-terminus by the formula: (G₂S)-(EA₃K)₄-(G₂S)-(EA₃K) 2-(G₂S)-(X) (SEQ ID NO: 36) in which X is a Sortase A recognition site including the amino acid sequence Leu-Pro-Glu-Thr-Gly (SEQ ID NO: 33).

In some embodiments, the flexible linker is a polypeptide flexible linker having a structure represented from N-terminus to C-terminus by the formula: (G₂S)-(EA₃K)₄-(G₂S)-(EA₃K) 2-(G₂S) (SEQ ID NO: 37).

In some embodiments, the flexible tether further includes a cleavable linker configured to cleave upon its activation by a trigger.

In some embodiments, the cleavable linker is a protease cleavage site configured to cleave upon its activation by a protease enzyme trigger, whereby upon activation of the protease cleavage site by the protease enzyme trigger, the protease cleavage site cleaves the blocking construct at the protease cleavage site.

In some embodiments, the cleavable linker is a photo-cleavable linker having an activation wavelength, the photo-cleavable linker configured to cleave upon its activation by exposure to light of the activation wavelength, whereby upon activation of the photo-cleavable linker by exposure to light of the activation wavelength, the photo-cleavable linker cleaves the blocking construct at the photo-cleavable linker.

In some embodiments, the cleavable linker is a photo-cleavable linker having an activation wavelength, the photo-cleavable linker configured to cleave upon its activation by exposure to light of the activation wavelength, whereby upon activation of the photo-cleavable linker, the photo-cleavable linker cleaves the blocking construct at the position of the photo-cleavable linker. In some embodiments, the photo-cleavable linker is a Fmoc cleavable linker. In other embodiments, the Fmoc cleavable linker is positioned at the N-terminus of the polypeptide. In further embodiments, the Fmoc cleavable linker is positioned at the C-terminus of the polypeptide.

In accordance with an embodiment, a kappa light chain-binding polypeptide is configured to have a binding interaction with the kappa light chain of an antigen binding domain derived from, or forming any portion of, an antibody or antibody fragment selected from, an immunoglobulin molecule, an IgA isotype antibody, an IgD isotype antibody, an IgE isotype antibody, an IgG isotype antibody, an IgM isotype antibody, a monospecific antibody, a bispecific antibody, a Fab fragment, a Fab′ fragment, an F(ab′)₂ fragment, an Fv fragment, a rigG fragment, a scFv fragment, a scFV-Fc fragment, and a minibody fragment.

In some embodiments, the antigen binding domain is derived from, or forms any portion of an immunoglobulin molecule. In some embodiments, the antigen binding domain is derived from, or forms any portion of an IgA isotype antibody. In some embodiments, the antigen binding domain is derived from, or forms any portion of an IgD isotype antibody. In some embodiments, the antigen binding domain is derived from, or forms any portion of an IgE isotype antibody. In some embodiments, the antigen binding domain is derived from, or forms any portion of an IgG isotype antibody. In some embodiments, the antigen binding domain is derived from, or forms any portion of an IgM isotype antibody. In some embodiments, the antigen binding domain is derived from, or forms any portion of a monospecific antibody. In some embodiments, the antigen binding domain is derived from, or forms any portion of a bispecific antibody. In some embodiments, the antigen binding domain is derived from, or forms any portion of a Fab fragment. In some embodiments, the antigen binding domain is derived from, or forms any portion of a Fab′ fragment. In some embodiments, the antigen binding domain is derived from, or forms any portion of an F(ab′)₂ fragment. In some embodiments, the antigen binding domain is derived from, or forms any portion of an Fv fragment. In some embodiments, the antigen binding domain is derived from, or forms any portion of a rigG fragment. In some embodiments, the antigen binding domain is derived from, or forms any portion of a scFv fragment. In some embodiments, the antigen binding domain is derived from, or forms any portion of a scFV-Fc fragment. In some embodiments, the antigen binding domain is derived from, or forms any portion of a minibody fragment.

In some embodiments, the antibody is selected from alemtuzumab, bevacizumab, cetuximab, edrecolomab, gemtuzumab, ibritumomab tiuxetan, matuzumab, panitumumab, rituximab, and trastuzumab.

In accordance with an embodiment, a blocked immunoglobulin complex includes an immunoglobulin crosslinked to a set of one or more blocking constructs. The immunoglobulin may be selected from an alemtuzumab, a bevacizumab, a cetuximab, a edrecolomab, a gemtuzumab, a ibritumomab tiuxetan, a matuzumab, a panitumumab, a rituximab, a trastuzumab, and an anti-FLAG antibody.

In some embodiments, the immunoglobulin is a alemtuzumab. In some embodiments, the immunoglobulin is a bevacizumab. In some embodiments, the immunoglobulin is a cetuximab. In some embodiments, the immunoglobulin is an edrecolomab. In some embodiments, the immunoglobulin is a gemtuzumab. In some embodiments, the immunoglobulin is an ibritumomab tiuxetan. In some embodiments, the immunoglobulin is a matuzumab. In some embodiments, the immunoglobulin is a panitumumab. In some embodiments, the immunoglobulin is a rituximab. In some embodiments, the immunoglobulin is a trastuzumab. In some embodiments, the immunoglobulin is an anti-FLAG antibody.

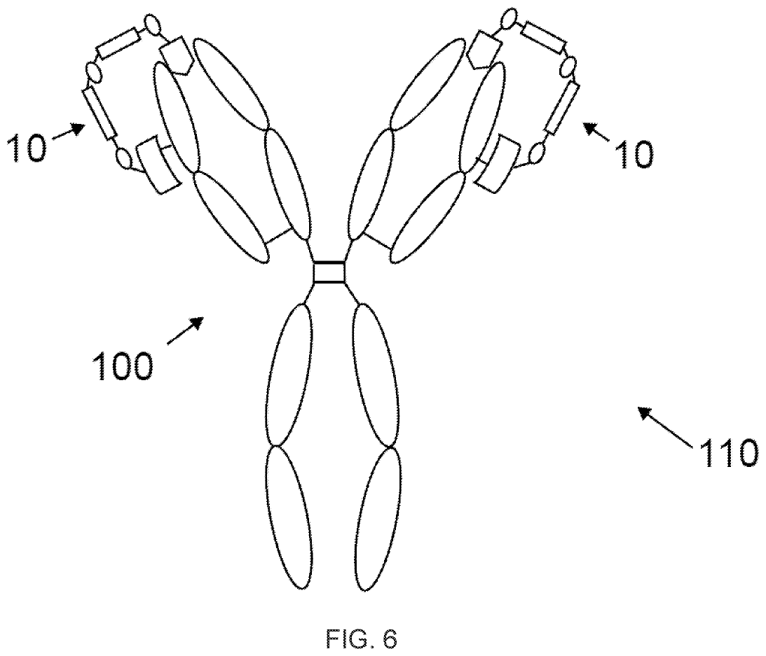

FIG. 6 is a graphical representation of an IgG Isotype immunoglobulin 100 (such as cetuximab or other anti-EGFR IgG isotype antibodies) crosslinked to a set of one or more blocking constructs 10 to form a blocked immunoglobulin complex 110.

In some embodiments, at least one blocking construct in a set of one or more blocking constructs includes a blocking construct in which the epitope of the blocking moiety of the blocking construct is an EGFR epitope including the amino acid sequence Gln-Gly-Gln-Ser-Gly-Gln-Cys-Ile-Ser-Pro-Arg-Gly-Cys-Pro-Asp-Gly-Pro-Tyr-Val-Met-Tyr (SEQ ID NO: 32).

In some embodiments, at least one blocking construct in a set of one or more blocking constructs is selected from a blocking construct in which the epitope of the blocking moiety of the blocking construct is a FLAG epitope including the amino acid sequence Asp-Tyr-Lys-Asp-Asp-Asp-Asp-Lys (SEQ ID NO: 31).

In some embodiments, the immunoglobulin includes an antigen binding domain antigen binding domain derived from, or forming any portion of, an antibody or antibody fragment selected from, an immunoglobulin molecule, an IgA isotype antibody, an IgD isotype antibody, an IgE isotype antibody, an IgG isotype antibody, an IgM isotype antibody, a monospecific antibody, a bispecific antibody, a Fab fragment, a Fab′ fragment, an F(ab′)₂ fragment, an Fv fragment, a rigG fragment, a scFv fragment, a scFV-Fc fragment, and a minibody fragment.

In some embodiments, the antigen binding domain is derived from, or forms any portion of an immunoglobulin molecule. In some embodiments, the antigen binding domain is derived from, or forms any portion of an IgA isotype antibody. In some embodiments, the antigen binding domain is derived from, or forms any portion of an IgD isotype antibody. In some embodiments, the antigen binding domain is derived from, or forms any portion of an IgE isotype antibody. In some embodiments, the antigen binding domain is derived from, or forms any portion of an IgG isotype antibody. In some embodiments, the antigen binding domain is derived from, or forms any portion of an IgM isotype antibody. In some embodiments, the antigen binding domain is derived from, or forms any portion of a monospecific antibody. In some embodiments, the antigen binding domain is derived from, or forms any portion of a bispecific antibody. In some embodiments, the antigen binding domain is derived from, or forms any portion of a Fab fragment. In some embodiments, the antigen binding domain is derived from, or forms any portion of a Fab′ fragment. In some embodiments, the antigen binding domain is derived from, or forms any portion of an F(ab′)₂ fragment. In some embodiments, the antigen binding domain is derived from, or forms any portion of an Fv fragment. In some embodiments, the antigen binding domain is derived from, or forms any portion of a rigG fragment. In some embodiments, the antigen binding domain is derived from, or forms any portion of a scFv fragment. In some embodiments, the antigen binding domain is derived from, or forms any portion of a scFV-Fc fragment. In some embodiments, the antigen binding domain is derived from, or forms any portion of a minibody fragment.

In accordance with an embodiment, a blocked immunoglobulin complex, includes a heavy chain including SEQ ID NO: 42; and a light chain including SEQ ID NO: 43; in which, the light chain is crosslinked to a blocking construct.

In accordance with an embodiment, a pharmaceutical composition includes a blocked immunoglobulin complex. In some embodiments, the immunoglobulin of the blocked immunoglobulin complex is cetuximab. In some embodiments, the pharmaceutical composition in which the immunoglobulin of the blocked immunoglobulin complex is cetuximab may be administered at an intravenous dosage of from 100 mg/m2 to 600 mg/m2 as a 120-minute intravenous infusion every two weeks (Q2W). In other embodiments, the dosage may be from 250 mg/m2 to 600 mg/m2 as a 120-minute intravenous infusion every two weeks (Q2W). In other embodiments, the dosage may be from 450 mg/m2 to 550 mg/m2 as a 120-minute intravenous infusion every two weeks (Q2W). In additional embodiments, the dosage may be from 500 mg/m2 as a 120-minute intravenous infusion every two weeks (Q2W).

In further embodiments, the pharmaceutical composition in which the immunoglobulin of the blocked immunoglobulin complex is cetuximab may be administered as an initial intravenous dose of from 250 mg/m2 to 600 mg/m2, followed by weekly intravenous doses of 250 mg/m2 for one or more weeks. In some embodiments, the initial intravenous dose may be administered at 400 mg/m2 to 550 mg/m2, followed by weekly intravenous doses of 250 mg/m2 for one or more weeks. In some embodiments, the initial intravenous dose is given at 400 mg/m2. In other embodiments, the initial intravenous dose is given at 500 mg/m2.

In still further embodiments, the pharmaceutical composition in which the immunoglobulin of the blocked immunoglobulin complex is cetuximab may be administered as an initial intravenous dose of from 500 mg/m2 to 1200 mg/m2, followed by weekly intravenous doses of 500 mg/m2 for one or more weeks. In some embodiments, the initial intravenous dose may be administered at 800 mg/m2 to 1100 mg/m2, followed by weekly intravenous doses of 500 mg/m2 for one or more weeks. In some embodiments, the initial intravenous dose is given at 800 mg/m2. In other embodiments, the initial intravenous dose is given at 1000 mg/m2.

In yet further embodiments, the pharmaceutical composition in which the immunoglobulin of the blocked immunoglobulin complex is cetuximab may be administered as an initial intravenous dose of from 1000 mg/m2 to 2400 mg/m2, followed by weekly intravenous doses of 1000 mg/m2 for one or more weeks. In some embodiments, the initial intravenous dose may be administered at 1600 mg/m2 to 2200 mg/m2, followed by weekly intravenous doses of 1000 mg/m2 for one or more weeks. In some embodiments, the initial intravenous dose is given at 1600 mg/m2. In other embodiments, the initial intravenous dose is given at 2000 mg/m2.

Provided herein is a pharmaceutical composition including a pharmaceutically or therapeutically effective amount of an antibody construct, as described herein, and a pharmaceutically acceptable carrier. In some embodiments, the pharmaceutical composition is intended for intravenous delivery. In others, the pharmaceutical composition is designed for infusion administration.

Pharmaceutical compositions including the herein described antibodies constructs may prepared by mixing the antibody constructs having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-Methylglucosamine (so-called “Meglumine”), galactosamine and neuraminic acid; and/or non-ionic surfactants such as Tween, Brij Pluronics, Triton-X or polyethylene glycol (PEG).

The pharmaceutical composition may be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization), however also solutions including antibacterial agents may be used for the production of pharmaceutical compositions for parenteral administration; see also Chen, Drug Dev Ind Pharm 18:1311-1354, 1992.

Exemplary antibody construct concentrations in the pharmaceutical composition may range from 1 mg/mL to 200 mg/ml or from 50 mg/mL to 200 mg/mL, or from 150 mg/mL to 200 mg/mL. For clarity reasons, it is emphasized that the concentrations as indicated herein relate to the concentration in a liquid or in a liquid that is accurately reconstituted from a solid form.

An aqueous formulation of the antibody construct may be prepared in a pH-buffered solution, e.g., at pH ranging from 4.0 to 7.0, or from 5.0 to 6.0, or alternatively 5.5. Examples of buffers that are suitable for a pH within this range include phosphate-, histidine-, citrate-, succinate-, acetate-buffers and other organic acid buffers. The buffer concentration can be from 1 mM to 100 mM, or from 5 mM to 50 mM, depending, e.g., on the buffer and the desired tonicity of the formulation.

A tonicity agent may be included in the antibody construct formulation to modulate the tonicity of the formulation. Exemplary tonicity agents include sodium chloride, potassium chloride, glycerin and any component from the group of amino acids, sugars as well as combinations thereof. Preferably the aqueous formulation is isotonic, although hypertonic or hypotonic solutions may be suitable. The term “isotonic” denotes a solution having the same tonicity as some other solution with which it is compared, such as physiological salt solution and the blood serum. Tonicity agents may be used in an amount of 5 mM to 350 mM, in particular in an amount of 105 mM to 305 mM.

A surfactant may also be added to the antibody construct formulation to reduce aggregation of the formulated antibody construct and/or minimize the formation of particulates in the formulation and/or reduce adsorption. Exemplary surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic)., sodium sulphate and dodecyl (SDS). Preferred polyoxyethylenesorbitan-fatty acid esters are polysorbate 20, (sold under the trademark Tween 20™) and polysorbate 80 (sold under the trademark Tween 80™). Preferred polyethylene-polypropylene copolymers are those sold under the names Pluronic® F68 or Poloxamer 188™. Preferred Polyoxyethylene alkyl ethers are those sold under the trademark Brij™. Exemplary concentrations of surfactant may range from 0.001% to 1% w/v.

A lyoprotectant may also be utilized to protect a labile active ingredient (e.g. a protein) against destabilizing conditions during the lyophilization process. For example, lyoprotectants include sugars (including glucose and sucrose); polyols (including mannitol, sorbitol and glycerol); and amino acids (including alanine, glycine and glutamic acid). Lyoprotectants are generally used in an amount of 10 mM to 500 mM.

In some embodiments, the formulation contains the above-identified agents (i.e. antibody construct, surfactant, buffer, stabilizer and/or tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In another embodiment, a preservative may be included in the formulation, e.g., at concentrations ranging from 0.001 to 2% (w/v).

In some embodiments, a pharmaceutically useful composition may include, per ml of composition, from 0.5 mg to 5 mg of antibody construct, from 6 mg to 10 mg sodium chloride, from 1 mg to 2.5 mg sodium phosphate dibasic heptahydrate, from 0.25 mg to 0.6 mg sodium phosphate monobasic monohydrate, and Water for Injection, USP at pH of from 7.0 to 7.4.

In some embodiments, a pharmaceutically useful composition may include pH 5.6 to 6.0 liquid for intravenous (IV) infusion, containing per ml of composition from 5 mg to 30 mg of antibody construct, from 4 mg to 7 mg sodium chloride, from 5 mg to 8 mg sodium acetate, and Water for Injection, USP.

In accordance with an embodiment, a pharmaceutical composition includes a pharmaceutical excipient and a blocked immunoglobulin complex.

In accordance with an embodiment, a method of treating cancer, includes administering a therapeutically effective amount of a blocked immunoglobulin complex to a subject in need thereof.

### Methods of Use

Provided herein are methods of treatment for cancers that overexpress EGFR, each of the methods including administering to a subject in need thereof a pharmaceutically or therapeutically useful amount of blocked immunoglobulin complexes as described herein that modulate cetuximab's binding affinity for EFGR.

Cancers overexpressing EGFR include, but are not limited to, non-small cell lung cancer, colorectal cancer, brain tumors, astrocytoma, esophageal cancer, cervical cancer, synovial carcinoma, breast cancer (including her2 positive breast cancer), gastric cancers (including gastro-esophageal cancers), ovarian cancer.

### Head and Neck Cancer

Pharmaceutical compositions including a blocked immunoglobulin complex as described herein in which the immunoglobulin of the blocked immunoglobulin complex is cetuximab (i.e., a cetuximab blocked immunoglobulin complex) may be used in a method of treatment of head and neck cancer in a subject, the method including administering to the subject in need thereof a pharmaceutically or therapeutically useful amount of the cetuximab blocked immunoglobulin complex. The methods herein for the treatment of head and neck cancer in a subject include first line treatment, second line treatment, locoregional head and neck cancer, and metastatic head and neck cancer. The methods include the treatment of head and neck cancers associated with high expression of EGFR, including hypopharyngeal cancer, laryngeal cancer, lip and oral cavity cancer, metastatic squamous neck cancer. Nasopharyngeal cancer, oropharyngeal cancer, paranasal sinus and nasal cavity cancer, and salivary gland cancer.

Another embodiment provides the use of such cetuximab blocked immunoglobulin complexes as described herein in a method of treatment of squamous cell carcinoma of the head and neck in a subject, the method including administering to the subject in need thereof a pharmaceutically or therapeutically useful amount of the cetuximab blocked immunoglobulin complex.

An additional embodiment provides a method of treatment of squamous cell carcinoma of the head and neck in a subject, the method including administering to the subject in need thereof: a pharmaceutically or therapeutically useful amount of the cetuximab blocked immunoglobulin complex; and a pharmaceutically or therapeutically useful amount of monalizumab.

A different embodiment provides a method of treatment of squamous cell carcinoma of the head and neck in a subject, the method including administering to the subject in need thereof: a pharmaceutically or therapeutically useful amount of the cetuximab blocked immunoglobulin complex; and a pharmaceutically or therapeutically useful amount of ficlatuzumab.

A different embodiment provides a method of treatment of squamous cell carcinoma of the head and neck in a subject, the method including administering to the subject in need thereof: a pharmaceutically or therapeutically useful amount of the cetuximab blocked immunoglobulin complex; and a pharmaceutically or therapeutically useful amount of palbociclib, or a pharmaceutically acceptable salt thereof.

A different embodiment provides a method of treatment of squamous cell carcinoma of the head and neck in a subject, the method including administering to the subject in need thereof: a pharmaceutically or therapeutically useful amount of the cetuximab blocked immunoglobulin complex; and a pharmaceutically or therapeutically useful amount of cabozantinib, or a pharmaceutically acceptable salt thereof.

A further embodiment provides a method of treatment of squamous cell carcinoma of the head and neck in a subject, the method including administering to the subject in need thereof: a pharmaceutically or therapeutically useful amount of the cetuximab blocked immunoglobulin complex; and a pharmaceutically or therapeutically useful amount of penpulimab.

A further embodiment provides a method of treatment of squamous cell carcinoma of the head and neck in a subject, the method including administering to the subject in need thereof: a pharmaceutically or therapeutically useful amount of the cetuximab blocked immunoglobulin complex; and a pharmaceutically or therapeutically useful amount of pembrolizumab.

A still further embodiment provides a method of treatment of squamous cell carcinoma of the head and neck in a subject, the method including administering to the subject in need thereof: a pharmaceutically or therapeutically useful amount of the cetuximab blocked immunoglobulin complex; and a pharmaceutically or therapeutically useful amount of 5-Fluorouracil (5-FU); and a pharmaceutically or therapeutically useful amount of an agent selected from the group of cisplatin and carboplatin, or a pharmaceutically acceptable salt thereof.

A yet further embodiment provides a method of treatment of squamous cell carcinoma of the head and neck in a subject, the method including administering to the subject in need thereof: a pharmaceutically or therapeutically useful amount of the cetuximab blocked immunoglobulin complex; and a pharmaceutically or therapeutically useful amount of paclitaxel, or a pharmaceutically acceptable salt thereof; and a pharmaceutically or therapeutically useful amount of carboplatin, or a pharmaceutically acceptable salt thereof.

Another embodiment provides the use of such constructs in which the antibody portion is cetuximab in a method of treatment of colon cancer, including metastatic colorectal cancer, in which the cancer cells express epidermal growth factor receptor (EGFR) protein, in a subject, the method including administering to the subject in need thereof a pharmaceutically or therapeutically useful amount of the cetuximab blocked immunoglobulin complex.

A further embodiment provides the use of such constructs in which the antibody portion is cetuximab in a method of treatment of RAS wild-type (WT) metastatic colorectal cancer in a subject, the method including administering to the subject in need thereof a pharmaceutically or therapeutically useful amount of the cetuximab blocked immunoglobulin complex.

An additional embodiment provides a method of treatment of RAS wild-type (WT) metastatic colorectal cancer in a subject, the method including administering to the subject in need thereof: a pharmaceutically or therapeutically useful amount of the cetuximab blocked immunoglobulin complex, as described herein; and a pharmaceutically or therapeutically useful amount of one or more chemotherapeutic agents selected from the group of oxaliplatin, irinotecan, regorafenib, trifluridin tipiracil (TAS-102), pembrolizumab, afatinib, tepotinib, leucovorin, 5-fluorouracil, capecitabine, bevacizumab, ziv-aflibercept, ramucirumab, panitumumab, leucovorin, and Trifluridine with tipiracil.

An additional embodiment provides a method of treatment of metastatic colorectal cancer in a subject, the method including administering to the subject in need thereof: a pharmaceutically or therapeutically useful amount of the cetuximab blocked immunoglobulin complex, as described herein; and a pharmaceutically or therapeutically useful amount of one or more anticancer agents selected from the group of leucovorin, 5-FU, and oxaliplatin, or a pharmaceutically acceptable salt thereof.

Another embodiment provides a method of treatment of metastatic colorectal cancer in a subject, the method including administering to the subject in need thereof: a pharmaceutically or therapeutically useful amount of the cetuximab blocked immunoglobulin complex, as described herein; and a pharmaceutically or therapeutically useful amount of one or more anticancer agents selected from the group of leucovorin, 5-FU, and irinotecan, or a pharmaceutically acceptable salt thereof.

Another embodiment provides a method of treatment of metastatic colorectal cancer in a subject, the method including administering to the subject in need thereof: a pharmaceutically or therapeutically useful amount of the cetuximab blocked immunoglobulin complex, as described herein; and a pharmaceutically or therapeutically useful amount of one or more anticancer agents selected from the group of capecitabine and oxaliplatin, or a pharmaceutically acceptable salt thereof.

Another embodiment provides a method of treatment of metastatic colorectal cancer in a subject, the method including administering to the subject in need thereof: a pharmaceutically or therapeutically useful amount of the cetuximab blocked immunoglobulin complex, as described herein; and a pharmaceutically or therapeutically useful amount of one or more anticancer agents selected from the group of leucovorin, 5-FU, oxaliplatin, and irinotecan, or a pharmaceutically acceptable salt thereof.

An additional embodiment provides a method of treatment of RAS wild-type (WT) metastatic colorectal cancer in a subject, the method including administering to the subject in need thereof: a pharmaceutically or therapeutically useful amount of the cetuximab blocked immunoglobulin complex, as described herein; and a pharmaceutically or therapeutically useful amount of afatinib, or a pharmaceutically acceptable salt thereof.

An additional embodiment provides a method of treatment of RAS wild-type (WT) metastatic colorectal cancer in a subject, the method including administering to the subject in need thereof: a pharmaceutically or therapeutically useful amount of the cetuximab blocked immunoglobulin complex, as described herein; and a pharmaceutically or therapeutically useful amount of tefotinib, or a pharmaceutically acceptable salt thereof.

An additional embodiment provides a method of treatment of colorectal cancer in a subject, the method including administering to the subject in need thereof: a pharmaceutically or therapeutically useful amount of the cetuximab blocked immunoglobulin complex, as described herein; and a pharmaceutically or therapeutically useful amount of encorafenib, or a pharmaceutically acceptable salt thereof.

Another embodiment provides a method of treatment of colorectal cancer in a subject, the method including administering to the subject in need thereof: a pharmaceutically or therapeutically useful amount of the cetuximab blocked immunoglobulin complex, as described herein; and a pharmaceutically or therapeutically useful amount of encorafenib, or a pharmaceutically acceptable salt thereof; and a pharmaceutically or therapeutically useful amount of binimetinib, or a pharmaceutically acceptable salt thereof.

An additional embodiment provides a method of treatment of RAS wild-type (WT) metastatic colorectal cancer in a subject, the method including administering to the subject in need thereof: a pharmaceutically or therapeutically useful amount of the blocked cetuximab antibody, as described herein; and a pharmaceutically or therapeutically useful amount of vemurafenib, or a pharmaceutically acceptable salt thereof; and a pharmaceutically or therapeutically useful amount of camrelizumab.

An additional embodiment provides a method of treatment of metastatic colorectal adenocarcinoma with mutant APC, mutant TP53 and mutant KRAS genes in a subject, the method including administering to the subject in need thereof a pharmaceutically or therapeutically useful amount of the cetuximab blocked immunoglobulin complex, as described herein.

Still another embodiment provides the use of such cetuximab blocked immunoglobulin complexes in a method of treatment of colon cancer, including metastatic colorectal cancer, in which the cancer cells contain at least one gene mutation selected from the group of a K-RAS (RAS) gene mutation, a RAF gene mutation, and a PI3K gene mutation in a subject, the method including administering to the subject in need thereof a pharmaceutically or therapeutically useful amount of the cetuximab blocked immunoglobulin complexes.

Non-limiting examples of K-RAS mutations of relevance to methods herein include G12A, G12C, G12D, G12R, G12S, G12V, G13A, G13C, G13D, G13S, G13V, A146P, A146T, A146V, Q61H, Q61L, Q61R, and K117N mutations.

A different embodiment provides a method of treating colon cancer with a K-RAS mutation present, including metastatic colon cancer with a K-RAS mutation present, in a subject, the method including administering to the subject: a pharmaceutically or therapeutically effective amount of the blocked cetuximab antibody, as described herein, and a pharmaceutically a pharmaceutically or therapeutically effective amount of panitumumab.

Yet another embodiment provides the use of such cetuximab blocked immunoglobulin complexes in a method of treatment of colon cancer, including metastatic colorectal cancer, in which the cancer cells overexpress EGFR ligand, in a subject, the method including administering to the subject in need thereof a pharmaceutically or therapeutically useful amount of the blocked cetuximab antibody.

An additional embodiment provides a method of treatment of metastatic colorectal cancer in a subject, the method including administering to the subject in need thereof: a pharmaceutically or therapeutically useful amount of the blocked cetuximab antibody, as described herein; a pharmaceutically or therapeutically useful amount of irinotecan, or a pharmaceutically acceptable salt thereof; a pharmaceutically or therapeutically useful amount of oxaliplatin, or a pharmaceutically acceptable salt thereof; and a pharmaceutically or therapeutically useful amount of 5-fluorouracil, or a pharmaceutically acceptable salt thereof.

In accordance with an embodiment, a method of modulating the binding activity of antigen binding domain includes: providing a set of one or more blocking constructs; and crosslinking the set of one or more blocking constructs to an antigen binding domain to thereby modulate the binding activity of the antigen binding domain. In some embodiments, the set of one or more blocking constructs is exposed to an ultraviolet light trigger to activate the cleavable linker of the blocking construct to disassociate the blocking moiety from the antigen binding domain to reduce the effective concentration of block moiety at the antigen binding domain and thereby modify the binding activity antigen binding domain to an antigen. In some embodiments, the ultraviolet light has a wavelength of 365 nm.

In accordance with an embodiment, a method for producing a kappa light chain-binding polypeptide includes: expressing a nucleic acid sequence encoding the kappa light chain-binding polypeptide amino acid sequence of a kappa light chain-binding polypeptide in cells to produce the kappa light chain-binding polypeptide; and extracting and purifying the produced kappa light chain-binding polypeptide.

In accordance with an embodiment, a method for producing a blocking construct includes: expressing a nucleic acid sequence encoding the amino acid sequence of a blocking construct as described herein in cells to produce the blocking construct; and extracting and purifying the produced blocking construct.

In accordance with an embodiment, a method for producing a blocked immunoglobulin complex includes: expressing a nucleic acid sequence encoding the amino acid sequence of the immunoglobulin of the blocked immunoglobulin complex in cells to produce the immunoglobulin; expressing a nucleic acid sequence encoding the amino acid sequence of a blocking construct as described herein in cells to produce the blocking construct; crosslinking the produced immunoglobulin to produce blocking constructs by exposing the produced immunoglobulin and blocking constructs to an ultraviolet light and thereby form a blocked immunoglobulin complex; and extracting and purifying the crosslinked blocked immunoglobulin complex.

In accordance with an embodiment, a kit for use in modifying the binding activity of an antigen binding domain includes components as described herein including a kappa light chain-binding polypeptide, a blocking construct, a blocked immunoglobulin complex, or a pharmaceutical composition. In some embodiments, the kit further includes instructions for combining the components.

Unless otherwise defined, all terms of art, notations and other scientific terminology used herein are intended to have the meanings commonly understood by those having skill in the art. In some cases, terms with commonly understood meanings are defined herein for clarity and the inclusion of such definitions should not necessarily be construed to represent a difference over what is generally understood in the art. The techniques and methods referenced to herein are generally understood in the art, such as, for example, the widely utilized molecular cloning methodologies described in Sambrook et al., Molecular Cloning: A Laboratory Manual 4th ed. (2012) Cold Springs Harbor Laboratory Press, Cold Springs Harbor, NY. As appropriate, procedures involving the use of commercially available kits and reagents are generally carried out in accordance with manufacturer-defined protocols and conditions unless otherwise noted.

The Exemplary Embodiments and Example(s) below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### EXEMPLARY EMBODIMENTS

1. A blocking construct for modulating binding activity of an antigen binding domain, the blocking construct including: a kappa light chain-binding polypeptide including a Protein L amino acid sequence in which at least one amino acid residue in the Protein L amino acid sequence is substituted by a photo-reactive crosslinker residue having an activation wavelength; da blocking moiety including an epitope configured to bind competitively to an antigen binding site of the antigen binding domain; and a flexible tether, operatively connecting the kappa light chain-binding polypeptide to the blocking moiety.

2. The blocking construct of embodiment 1, wherein the antigen binding domain is specific for the FLAG-tag, and the epitope of the blocking moiety includes the amino acid sequence Asp-Tyr-Lys-Asp-Asp-Asp-Asp-Lys (SEQ ID NO: 31).

3. A kappa light chain-binding polypeptide, including: a set of one or more crosslinker kappa light chain-binding domains, in which a crosslinker kappa light chain-binding domain in the set includes a Protein L amino acid sequence in which at least one amino acid residue in the Protein L amino acid sequence is substituted by a reactive crosslinker, such as a photo-reactive crosslinker residue having an activation wavelength.

4. The polypeptide of embodiment 3, in which the Protein L amino acid sequence is selected from the Protein L amino acid sequence set forth in any one of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10, in which the amino acid residue being substituted corresponds to position 33 of the selected amino acid sequence.

5. The polypeptide of embodiment 3, in which a crosslinker kappa light chain-binding domain in the set includes the Protein L amino acid sequence set forth in in any one of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11.

6. The polypeptide of embodiment 3, in which the photo-reactive crosslinker residue is selected from a 4-benzoyl-L-phenylalanine (BpA) residue, a (2R)-2-amino-3-fluoro-3-(4-((2-nitrobenzyl)oxy) phenyl) propanoic acid residue (FnbY), a p-benzoyl-L-phenylalanine (pBpA), a n-(Fluoroacetyl)phenylalanine residue, a p-2′-fluoroacetyl-phenylalanine (Ffact) residue, a p-azidophenylalanine (pAzF), a p-vinylsulfonamido-(S)-phenylalanine residue, and a p-isothiocyanate phenylalanine (pNCSF) residue.

7. The polypeptide of embodiment 3, in which the activation wavelength of the photo-reactive crosslinker residue is 365 nm.

8. The polypeptide of embodiment 3, in which a crosslinker kappa light chain-binding domain in the set includes a Protein L amino acid sequence selected from the Protein L amino acid sequence set forth in any one of: SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, or SEQ ID NO: 22.

9. The polypeptide of embodiment 3, in which a crosslinker kappa light chain-binding domain in the set includes a Protein L polypeptide structure represented from N-terminus to C-terminus by the formula: 1-L1-B2-a-L2-3-L3-β4, in which: 31 is a first beta-sheet motif including a Protein L amino acid sequence selected from the Protein L amino acid sequence corresponding to positions 1 to 9 as set forth in: SEQ ID NO: 1; or SEQ ID NO: 1, in which the amino acid residue corresponding to position 6 as set forth in SEQ ID NO: 1 is substituted by alanine; or SEQ ID NO: 1, in which the amino acid residues corresponding to positions 1, 6, 8, and 9 as set forth in SEQ ID NO: 1 are substituted by, respectively, glutamine, glutamate, isoleucine, and tyrosine; 32 is a second beta-sheet motif including a Protein L amino acid sequence selected from the Protein L amino acid sequence corresponding to positions 15 to 23 as set forth in: SEQ ID NO: 1; or SEQ ID NO: 1, in which the amino acid residue corresponding to position 15 as set forth in SEQ ID NO: 1 is substituted by threonine; or SEQ ID NO: 1, in which the amino acid residues corresponding to positions 15 and 17 as set forth in SEQ ID NO: 1 are substituted by, respectively, threonine and asparagine; or SEQ ID NO: 1, in which the amino acid residues corresponding to positions 15 and 19 as set forth in SEQ ID NO: 1 are substituted by, respectively, valine and threonine; 33 is a third beta-sheet motif including a Protein L amino acid sequence selected from the Protein L amino acid sequence corresponding to positions 45 to 50 as set forth in: SEQ ID NO: 1; or SEQ ID NO: 1, in which the amino acid residues corresponding to positions 47, 49, and 50 as set forth in SEQ ID NO: 1 are substituted by, respectively, valine, valine, and alanine; 34 is a fourth beta-sheet motif including a Protein L amino acid sequence selected from the Protein L amino acid sequence corresponding to positions 55 to 61 as set forth in: SEQ ID NO: 1; or SEQ ID NO: 1, in which the amino acid residues corresponding to positions 55 and 56 as set forth in SEQ ID NO: 1 are substituted by, respectively, threonine and leucine; or SEQ ID NO: 1, in which the amino acid residues corresponding to positions 55 and 56 as set forth in SEQ ID NO: 1 are substituted by, respectively, threonine and isoleucine; or SEQ ID NO: 1, in which the amino acid residues corresponding to positions 55, 56, and 59 as set forth in SEQ ID NO: 1 are substituted by, respectively, threonine, isoleucine, and arginine; α is an alpha helix motif including a Protein L amino acid sequence selected from the Protein L amino acid sequence corresponding to positions 24 to 39 as set forth in: SEQ ID NO: 1; or SEQ ID NO: 1, in which the amino acid residue corresponding to position 25 as set forth in SEQ ID NO: 1 is substituted by alanine; or SEQ ID NO: 1, in which the amino acid residues corresponding to positions 25 and 39 as set forth in SEQ ID NO: 1 are substituted by, respectively, alanine and serine; or SEQ ID NO: 1, in which the amino acid residues corresponding to positions 25, 26, 28, 29, 30, 37, and 39 as set forth in SEQ ID NO: 1 are substituted by, respectively, alanine, lysine, valine, serine, aspartate, alanine, and lysine; or SEQ ID NO: 1, in which the amino acid residues corresponding to positions 26, 29, 37, and 39 as set forth in SEQ ID NO: 1 are substituted by, respectively, lysine, serine, threonine, and lysine; or SEQ ID NO: 1, in which the amino acid residue corresponding to position 30 as set forth in SEQ ID NO: 1 is substituted by lysine; or SEQ ID NO: 1, in which the amino acid residues corresponding to positions 30 and 36 as set forth in SEQ ID NO: 1 are substituted by, respectively, lysine and asparagine; or SEQ ID NO: 1, in which the amino acid residue corresponding to position 33 as set forth in the selected amino acid sequence is substituted by a photo-reactive crosslinker residue having an activation wavelength, the photo-reactive crosslinker residue; or SEQ ID NO: 1, in which the amino acid residues corresponding to positions 37 and 39 as set forth in SEQ ID NO: 1 are substituted by, respectively, alanine and lysine; L1 is an amino acid linker including a Protein L amino acid sequence selected from the Protein L amino acid sequence corresponding to positions 10 to 14 as set forth in: SEQ ID NO: 1; or SEQ ID NO: 1, in which the amino acid residue corresponding to position 10 as set forth in SEQ ID NO: 1 is substituted by tyrosine; or SEQ ID NO: 1, in which the amino acid residue corresponding to position 11 as set forth in SEQ ID NO: 1 is substituted by glutamate; or SEQ ID NO: 1, in which the amino acid residue corresponding to position 12 as set forth in SEQ ID NO: 1 is substituted by asparagine; or SEQ ID NO: 1, in which the amino acid residue corresponding to position 13 as set forth in SEQ ID NO: 1 is substituted by serine; L2 is an amino acid linker including a Protein L amino acid sequence selected from the Protein L amino acid sequence corresponding to positions 40 to 44 as set forth in: SEQ ID NO: 1; or SEQ ID NO: 1, in which the amino acid residue corresponding to position 41 as set forth in SEQ ID NO: 1 is substituted by aspartate; or SEQ ID NO: 1, in which the amino acid residue corresponding to position 41 as set forth in SEQ ID NO: 1 is substituted by glutamate; or SEQ ID NO: 1, in which the amino acid residues corresponding to positions 41 and 44 as set forth in SEQ ID NO: 1 are substituted by, respectively, glutamate and lysine; or SEQ ID NO: 1, in which the amino acid residues corresponding to positions 41, 42, and 44 as set forth in SEQ ID NO: 1 are substituted by, respectively, glutamate, histidine, and lysine; and L3 is an amino acid linker including a Protein L amino acid sequence selected from the Protein L amino acid sequence corresponding to positions 51 to 54 as set forth in: SEQ ID NO: 1; or SEQ ID NO: 1, in which the amino acid residue corresponding to position 52 as set forth in SEQ ID NO: 1 is substituted by lysine.

10. The polypeptide of embodiment 3, in which β1 is a first beta-sheet motif including a Protein L amino acid sequence selected from the Protein L amino acid sequence corresponding to positions 1 to 9 as set forth in SEQ ID NO 1.

11. The polypeptide of embodiment 10, in which: the amino acid residue corresponding to position 6 as set forth in SEQ ID NO: 1 is substituted by alanine; or the amino acid residues corresponding to positions 1, 6, 8, and 9 as set forth in SEQ ID NO: 1 are substituted by, respectively, glutamine, glutamate, isoleucine, and tyrosine.

12. The polypeptide of embodiment 3, in which 32 is a second beta-sheet motif including a Protein L amino acid sequence selected from the Protein L amino acid sequence corresponding to positions 15 to 23 as set forth in SEQ ID NO: 1.

13. The polypeptide of embodiment 12, in which: the amino acid residue corresponding to position 15 as set forth in SEQ ID NO: 1 is substituted by threonine or the amino acid residues corresponding to positions 15 and 17 as set forth in SEQ ID NO: 1 are substituted by, respectively, threonine and asparagine; or the amino acid residues corresponding to positions 15 and 19 as set forth in SEQ ID NO: 1 are substituted by, respectively, valine and threonine.

14. The polypeptide of embodiment 3, in which 33 is a third beta-sheet motif including a Protein L amino acid sequence selected from the Protein L amino acid sequence corresponding to positions 45 to 50 as set forth in SEQ ID NO: 1

15. The polypeptide of embodiment 14, in which the amino acid residues corresponding to positions 47, 49, and 50 as set forth in SEQ ID NO: 1 are substituted by, respectively, valine, valine, and alanine.

16. The polypeptide of embodiment 3, in which B4 is a fourth beta-sheet motif including a Protein L amino acid sequence selected from the Protein L amino acid sequence corresponding to positions 55 to 61 as set forth in SEQ ID NO: 1.

17. The polypeptide of embodiment 16, in which: the amino acid residues corresponding to positions 55 and 56 as set forth in SEQ ID NO: 1 are substituted by, respectively, threonine and leucine; or the amino acid residues corresponding to positions 55 and 56 as set forth in SEQ ID NO: 1 are substituted by, respectively, threonine and isoleucine; or the amino acid residues corresponding to positions 55, 56, and 59 as set forth in SEQ ID NO: 1 are substituted by, respectively, threonine, isoleucine, and arginine.

18. The polypeptide of embodiment 3, in which α is an alpha helix motif including a Protein L amino acid sequence selected from the Protein L amino acid sequence corresponding to positions 24 to 39 as set forth in SEQ ID NO: 1.

19. The polypeptide of embodiment 18, in which: the amino acid residue corresponding to position 25 as set forth in SEQ ID NO: 1 is substituted by alanine; or the amino acid residues corresponding to positions 25 and 39 as set forth in SEQ ID NO: 1 are substituted by, respectively, alanine and serine; or the amino acid residues corresponding to positions 25, 26, 28, 29, 30, 37, and 39 as set forth in SEQ ID NO: 1 are substituted by, respectively, alanine, lysine, valine, serine, aspartate, alanine, and lysine; or the amino acid residues corresponding to positions 26, 29, 37, and 39 as set forth in SEQ ID NO: 1 are substituted by, respectively, lysine, serine, threonine, and lysine; or the amino acid residue corresponding to position 30 as set forth in SEQ ID NO: 1 is substituted by lysine; or the amino acid residues corresponding to positions 30 and 36 as set forth in SEQ ID NO: 1 are substituted by, respectively, lysine and asparagine; or the amino acid residues corresponding to positions 30 and 36 as set forth in SEQ ID NO: 1 are substituted by, respectively, lysine and asparagine; or the amino acid residue corresponding to position 33 as set forth in the selected amino acid sequence is substituted by a photo-reactive crosslinker residue having an activation wavelength; or the amino acid residues corresponding to positions 37 and 39 as set forth in SEQ ID NO: 1 are substituted by, respectively, alanine and lysine.

20. The polypeptide of embodiment 3, in which L1 is an amino acid linker including a Protein L amino acid sequence selected from the Protein L amino acid sequence corresponding to positions 10 to 14 as set forth in SEQ ID NO: 1.

21. The polypeptide of embodiment 20, in which: the amino acid residue corresponding to position 10 as set forth in SEQ ID NO: 1 is substituted by tyrosine; or the amino acid residue corresponding to position 11 as set forth in SEQ ID NO: 1 is substituted by glutamate; or the amino acid residue corresponding to position 12 as set forth in SEQ ID NO: 1 is substituted by asparagine; or the amino acid residue corresponding to position 13 as set forth in SEQ ID NO: 1 is substituted by serine.

22. The polypeptide of embodiment 3, in which L2 is an amino acid linker including a Protein L amino acid sequence selected from the Protein L amino acid sequence corresponding to positions 40 to 44 as set forth in SEQ ID NO: 1.

23. The polypeptide of embodiment 22, in which: the amino acid residue corresponding to position 41 as set forth in SEQ ID NO: 1 is substituted by aspartate; or the amino acid residue corresponding to position 41 as set forth in SEQ ID NO: 1 is substituted by glutamate; or the amino acid residues corresponding to positions 41 and 44 as set forth in SEQ ID NO: 1 are substituted by, respectively, glutamate and lysine; or the amino acid residues corresponding to positions 41 and 44 as set forth in SEQ ID NO: 1 are substituted by, respectively, glutamate and lysine.

24. The polypeptide of embodiment 3, in which L3 is an amino acid linker including a Protein L amino acid sequence selected from the Protein L amino acid sequence corresponding to positions 51 to 54 as set forth in SEQ ID NO: 1.

25. The polypeptide of embodiment 3, in which L3 is an amino acid linker including a Protein L amino acid sequence selected from the Protein L amino acid sequence corresponding to positions 51 to 54 as set forth in SEQ ID NO: 1, in which the amino acid residue corresponding to position 52 as set forth in SEQ ID NO: 1 is substituted by lysine.

26. The polypeptide of embodiment 3, in which a crosslinker kappa light chain-binding domain in the set includes an engineered Protein L kappa light chain-binding domain including a crosslinker alpha helix motif having a structure represented from N-terminus to C-terminus by the amino acid sequence set forth in any of: Phe-Glu-Glu-Ala-Thr-Ala-Glu-Ala-Tyr-X-Tyr-Ala-Asp-Leu-Leu-Ala (SEQ ID NO: 23); Phe-Ala-Lys-Ala-Val-Ser-Asp-Ala-Tyr-X-Tyr-Ala-Asp-Ala-Leu-Lys (SEQ ID NO: 24); Phe-Glu-Glu-Ala-Thr-Ala-Lys-Ala-Tyr-X-Tyr-Ala-Asp-Leu-Leu-Ala (SEQ ID NO: 25); Phe-Glu-Glu-Ala-Thr-Ala-Lys-Ala-Tyr-X-Tyr-Ala-Asn-Leu-Leu-Ala (SEQ ID NO: 26); Phe-Glu-Lys-Ala-Thr-Ser-Glu-Ala-Tyr-X-Tyr-Ala-Asp-Thr-Leu-Lys (SEQ ID NO: 27); Phe-Glu-Glu-Ala-Thr-Ala-Glu-Ala-Tyr-X-Tyr-Ala-Asp-Ala-Leu-Lys (SEQ ID NO: 28); Phe-Ala-Glu-Ala-Thr-Ala-Glu-Ala-Tyr-X-Tyr-Ala-Asp-Leu-Leu-Ala (SEQ ID NO: 29); and Phe-Ala-Glu-Ala-Thr-Ala-Glu-Ala-Tyr-X-Tyr-Ala-Asp-Leu-Leu-Ser (SEQ ID NO: 30), and in which the amino acid residue corresponding to position X is substituted by the photo-reactive crosslinker residue.

27. The polypeptide of embodiment 3, in which a crosslinker kappa light chain-binding domain in the set includes an engineered Protein L kappa light chain-binding domain selected from a domain C*, a domain C₁, a domain C₂, a domain C₃, a domain C₄, a domain B1, a domain B2, a domain B3, a domain B4, and a domain B5, in which the selected Protein L kappa light chain-binding domain includes the photo-reactive crosslinker residue.

28. The polypeptide of embodiment 3, in which the engineered Protein L kappa light chain-binding domain is: a domain C* including a photo-reactive crosslinker residue; a domain C₁ including a photo-reactive crosslinker residue; a domain C₂ including a photo-reactive crosslinker residue; a domain C₃ including a photo-reactive crosslinker residue; a domain C₄ including a photo-reactive crosslinker residue; a domain B1 including a photo-reactive crosslinker residue; a domain B2 including a photo-reactive crosslinker residue; a domain B3 including a photo-reactive crosslinker residue; a domain B4 including a photo-reactive crosslinker residue; or a domain B5 including a photo-reactive crosslinker residue.

29. The polypeptide of embodiment 3, in which the (photo-) reactive crosslinker residue is selected from a 4-benzoyl-L-phenylalanine (BpA) residue, a (2R)-2-amino-3-fluoro-3-(4-((2-nitrobenzyl)oxy) phenyl) propanoic acid residue (FnbY), a p-benzoyl-L-phenylalanine (pBpA), a n-(Fluoroacetyl)phenylalanine residue, a p-2′-fluoroacetyl-phenylalanine (Ffact) residue, a p-azidophenylalanine (pAzF), a p-vinylsulfonamido-(S)-phenylalanine residue, and a p-isothiocyanate phenylalanine (pNCSF) residue.

30. The polypeptide of embodiment 29, in which the activation wavelength of the photo-reactive crosslinker residue is 365 nm.

31. A blocking construct for modulating the binding activity of an antigen binding domain, the blocking construct including: the kappa light chain-binding polypeptide of any of embodiments 3-30; which is operatively connected via a flexible tether to a blocking moiety that is configured to bind to antigen binding site of the antigen binding domain.

32. A blocking construct for modulating the binding activity of an antigen binding domain, the blocking construct including: a kappa light chain-binding polypeptide including: a set of one or more crosslinker kappa light chain-binding domains, in which a crosslinker kappa light chain-binding domain in the set includes a Protein L amino acid sequence in which at least one amino acid residue in the Protein L amino acid sequence is substituted by a photo-reactive crosslinker residue having an activation wavelength; which is operatively connected via a flexible tether to a blocking moiety that is configured to bind to antigen binding site of the antigen binding domain.

33. The blocking construct of embodiment 1 or embodiment 32, in which: the kappa light chain-binding polypeptide is configured to have, when in the proximity of a kappa light chain of an antigen binding domain, a binding interaction with the kappa light chain and thereby form a non-covalent bond between the blocking construct and the antigen binding domain, and, upon exposure of a photo-reactive crosslinker residue of a kappa light chain-binding domain forming the non-covalent bond to light of its activation wavelength, to activate the photo-reactive crosslinker residue and crosslink the kappa light chain-binding domain forming the non-covalent bond to the kappa light chain and thereby form a covalent bond between the blocking construct and antigen binding domain; the blocking moiety includes an epitope configured to competitively bind to an antigen binding site of the antigen binding domain; and the flexible tether includes a flexible linker operatively connected at a proximal end to the kappa light chain-binding polypeptide and at a distal end to the blocking moiety, the flexible linker configured to have an end-to-end length to tether the blocking moiety at a sufficient movement radius for the blocking moiety to establish an intramolecular binding interaction between its epitope and the antigen binding site and to establish an effective concentration of the blocking moiety at the antigen binding site, thereby to facilitate the competitive binding of the blocking moiety at the antigen binding site and modulate the binding activity of the antigen binding domain.

34. The blocking construct of embodiment 1 or embodiment 32, in which the blocking moiety includes a polypeptide, a oligonucleotide, a glycoprotein, a fusion protein, an engineered protein, or any fragment or combination thereof.

34. The blocking construct of embodiment 1 or embodiment 32, in which the blocking moiety further includes a cleavable linker configured to cleave upon its activation by a trigger.

35. The blocking construct of embodiment 34, in which the blocking moiety is: a polypeptide blocking moiety, and the cleavable linker is a protease cleavage site configured to cleave upon its activation by a protease enzyme trigger, whereby upon activation of the protease cleavage site by the protease enzyme trigger, the protease cleavage site cleaves the blocking construct at the protease cleavage site; or a polypeptide, and the cleavable linker is a photo-cleavable linker having an activation wavelength, the photo-cleavable linker configured to cleave upon its activation by exposure to light of the activation wavelength, whereby upon activation of the photo-cleavable linker, the photo-cleavable linker cleaves the blocking construct at the position of photo-cleavable linker.

36. The blocking construct of embodiment 35, in which the photo-cleavable linker (if present) is a Fmoc cleavable linker.

37. The blocking construct of embodiment 36, in which the Fmoc cleavable linker is positioned: at the N-terminus of the polypeptide; or at the C-terminus of the polypeptide.

38. The blocking construct of embodiment 1 or embodiment 32, in which the blocking construct is crosslinked to an antigen binding domain, whereby, upon activation of the cleavable linker by the trigger, the cleavable linker cleaves the blocking construct at the cleavable linker to dissociate the epitope of the blocking moiety from the blocking construct and thereby decrease the effective concentration of the blocking moiety at the antigen binding site to further modulate the binding activity of the antigen binding domain.

39. The blocking construct of embodiment 1 or embodiment 32, in which the epitope of the blocking moiety is selected from any of the group consisting of: a FLAG epitope including the amino acid sequence Asp-Tyr-Lys-Asp-Asp-Asp-Asp-Lys (SEQ ID NO: 31); and a EGFR epitope including the amino acid sequence Gln-Gly-Gln-Ser-Gly-Gln-Cys-Ile-Ser-Pro-Arg-Gly-Cys-Pro-Asp-Gly-Pro-Tyr-Val-Met-Tyr (SEQ ID NO: 32).

40. The blocking construct of embodiment 1 or embodiment 32, in which the epitope of the blocking moiety is: a FLAG epitope including the amino acid sequence Asp-Tyr-Lys-Asp-Asp-Asp-Asp-Lys (SEQ ID NO: 31); or an EGFR epitope including the amino acid sequence Gln-Gly-Gln-Ser-Gly-Gln-Cys-Ile-Ser-Pro-Arg-Gly-Cys-Pro-Asp-Gly-Pro-Tyr-Val-Met-Tyr (SEQ ID NO: 32).

41. The blocking construct of embodiment 1 or embodiment 32, in which the kappa light chain-binding polypeptide, the blocking moiety, or the flexible tether further include a conjugation moiety.

42. The blocking construct of embodiment 41, wherein the conjugation moiety includes a sortase recognition site including the amino acid sequence Leu-Pro-Glu-Thr-Gly (SEQ ID NO: 33), or a click chemistry residue.

43. The blocking construct of embodiment 1 or embodiment 32, in which the flexible tether includes at least one of: a polypeptide flexible linker having a structure represented from N-terminus to C-terminus by the formula: (G₄S)-(EA₃K)₄-(G₄S)-(EA₃K)₄-(G₄S)-(X) (SEQ ID NO: 34), in which X is a Sortase A recognition site including the amino acid sequence: Leu-Pro-Glu-Thr-Gly (SEQ ID NO: 33); a polypeptide flexible linker having a structure represented from N-terminus to C-terminus by the formula: (G₄S)-(EA₃K)₄-(G₄S)-(EAAAK)₄-(G₄S) (SEQ ID NO: 35); a polypeptide flexible linker having a structure represented from N-terminus to C-terminus by the formula: (G₂S)-(EA₃K)₄-(G₂S)-(EA₃K)₄-(G₂S)-(X) (SEQ ID NO: 36), in which X is a Sortase A recognition site including the amino acid sequence: Leu-Pro-Glu-Thr-Gly (SEQ ID NO: 33); and a polypeptide flexible linker having a structure represented from N-terminus to C-terminus by the formula: (G₂S)-(EA₃K) 2-(G₂S)-(EA₃K)₄-(G₂S) (SEQ ID NO: 37).

44. The blocking construct of embodiment 1 or embodiment 32, in which the flexible linker includes at least one of: a polypeptide flexible linker having a structure represented from N-terminus to C-terminus by the formula: (G₄S)-(EA₃K)₄-(G₄S)-(EA₃K)₄-(G₄S)-(X) (SEQ ID NO: 34), in which X is a Sortase A recognition site including the amino acid sequence: Leu-Pro-Glu-Thr-Gly (SEQ ID NO: 33); a polypeptide flexible linker having a structure represented from N-terminus to C-terminus by the formula: (G₄S)-(EA₃K)₄-(G₄S)-(EAAAK)₄-(G₄S) (SEQ ID NO: 35); a polypeptide flexible linker having a structure represented from N-terminus to C-terminus by the formula: (G₂S)-(EA₃K)₄-(G₂S)-(EA₃K)₄-(G₂S)-(X) (SEQ ID NO: 36) in which X is a Sortase A recognition site including the amino acid sequence Leu-Pro-Glu-Thr-Gly (SEQ ID NO: 33); or a polypeptide flexible linker having a structure represented from N-terminus to C-terminus by the formula: (G₂S)-(EA₃K)₄-(G₂S)-(EA₃K)₄-(GYS) (SEQ ID NO: 37).

45. The blocking construct of embodiment 1 or embodiment 32, in which the flexible tether further includes a cleavable linker configured to cleave upon its activation by a trigger.

46. The blocking construct of embodiments 45, in which the cleavable linker includes: a protease cleavage site configured to cleave upon its activation by a protease enzyme trigger, whereby upon activation of the protease cleavage site by the protease enzyme trigger, the protease cleavage site cleaves the blocking construct at the protease cleavage site; or a photo-cleavable linker having an activation wavelength, the photo-cleavable linker configured to cleave upon its activation by exposure to light of the activation wavelength, whereby upon activation of the photo-cleavable linker, the photo-cleavable linker cleaves the blocking construct at the position of the photo-cleavable linker.

47. The blocking construct of embodiment 46, in which the photo-cleavable linker (if present) is a Fmoc cleavable linker.

48. The blocking construct of embodiment 47, in which the Fmoc cleavable linker is positioned at: the N-terminus of the polypeptide; or the C-terminus of the polypeptide.

49. The blocking construct of embodiment 1 or embodiment 32, in which the activation wavelength of the photo-cleavable linker is 365 nm.

50. The blocking construct of embodiment 1 or embodiment 32, in which the flexible tether includes a flexible portion and a rigid portion.

51. The blocking construct of embodiment 50, in which the flexible tether includes one or more repeating motifs of the structure (X—Y)n, in which X and Y are, respectively a flexible portion operatively connected to a rigid portion, and n is the number repeats.

52. The blocking construct of embodiment 51, in which n equals one, two, three, four, five, six, seven, eight, nine, ten, eleven, twelve, thirteen, fourteen, fifteen, sixteen, seventeen, eighteen, nineteen, or twenty.

53. The blocking construct of embodiment 51, in which the flexible portion is one of: a (G₂S) flexible portion including the amino acid sequence Gly-Gly-Ser; a (G3S) flexible portion including the amino acid sequence Gly-Gly-Gly-Ser; or a (G₄S) flexible portion including the amino acid sequence Gly-Gly-Gly-Gly-Ser (SEQ ID NO: 84).

54. The construct of embodiment 501, in which the rigid portion is a (EA₃K) 4 rigid portion including the amino acid sequence Glu-Ala-Ala-Ala-Lys-Glu-Ala-Ala-Ala-Lys-Glu-Ala-Ala-Ala-Lys-Glu-Ala-Ala-Ala-Lys (SEQ ID NO: 85).

55. The blocking construct of embodiment 1 or embodiment 32, in which the end-to-end length of the flexible tether is configured to be of from 1.0 angstrom (Å) to 5.0 Å, of from 5.0 Å to 10.0 Å, of from 10.0 Å to 15.0 Å, of from 15.0 Å to 20.0 Å, of from 20.0 Å to 25.0 Å, of from 25.0 Å to 30.0 Å, of from 30.0 Å to 35.0 Å, of from 35.0 Å to 40.0 Å, of from 45.0 Å to 50.0 Å, of from 50.0 Å to 55.0 Å, of from 55.0 Å to 60.0 Å, of from 60.0 Å to 65.0 Å, of from 65.0 Å to 70.0 Å, of from 75.0 Å to 80.0 Å, of from 80.0 Å to 85.0 Å, of from 85.0 Å to 90.0 Å, of from 95.0 Å to 100.0 Å, of from 105.0 Å to 110.0 Å, of from 115.0 Å to 120.0 Å, of from 125.0 Å to 130.0 Å, of from 135.0 Å to 140.0 Å, of from 140.0 Å to 145.0 Å, and of from 145.0 Å to 150.0 Å.

56. The blocking construct of embodiment 1 or embodiment 32, in which the rigid portion has a persistence length of from 1.0 angstrom (Å) to 2.0 Å, of from 2.0 Å to 3.0 Å, of from 2.0 Å to 3.0 Å, of from 3.0 Å to 4.0 Å, of from 4.0 Å to 5.0 Å, of from 6.0 Å to 7.0 Å, of from 7.0 Å to 8.0 Å, of from 8.0 Å to 9.0 Å, of from 9.0 Å to 10.0 Å, of from 10.0 Å to 11.0 Å, of from 12.0 Å to 13.0 Å, of from 13.0 Å to 14.0 Å, of from 14.0 Å to 15.0 Å, of from 16.0 Å to 17.0 Å, of from 17.0 Å to 18.0 Å, of from 18.0 Å to 19.0 Å, and of from 19.0 Å to 20.0 Å.

57. The kappa light chain-binding polypeptide of embodiment 3, in which the kappa light chain-binding polypeptide is configured to have a binding interaction with the kappa light chain of an antigen binding domain derived from, or forming any portion of, an antibody or antibody fragment selected from, an immunoglobulin, an IgA isotype antibody, an IgD isotype antibody, an IgE isotype antibody, an IgG isotype antibody, an IgM isotype antibody, a monospecific antibody, a bispecific antibody, a Fab fragment, a Fab′ fragment, an F(ab′)₂ fragment, an Fv fragment, a rigG fragment, a scFv fragment, a scFV-Fc fragment, and a minibody fragment.

58. The blocking construct of embodiment 1 or embodiment 32, in which the antigen binding domain is derived from, or forms any portion of an antibody selected from an alemtuzumab, a bevacizumab, a cetuximab, an edrecolomab, a gemtuzumab, an ibritumomab tiuxetan, a matuzumab, a panitumumab, a rituximab, and a trastuzumab.

59. A blocked immunoglobulin complex including: an immunoglobulin crosslinked to a set of one or more blocking constructs.

60. The blocked immunoglobulin complex of embodiment 59, in which the immunoglobulin is an antibody selected from an alemtuzumab, a bevacizumab, a cetuximab, an edrecolomab, a gemtuzumab, an ibritumomab tiuxetan, a matuzumab, a panitumumab, a rituximab, a trastuzumab, and an anti-FLAG antibody.

61. The blocked immunoglobulin complex of embodiment 60, in which a blocking construct in the set is selected from: any of the blocking constructs of embodiments 31-56 or 58, in which the epitope of the blocking moiety of the blocking construct is an EGFR epitope including the amino acid sequence Gln-Gly-Gln-Ser-Gly-Gln-Cys-Ile-Ser-Pro-Arg-Gly-Cys-Pro-Asp-Gly-Pro-Tyr-Val-Met-Tyr (SEQ ID NO: 32); or any of the blocking constructs of embodiments 1, 31-56, or 58, in which the epitope of the blocking moiety of the blocking construct is a FLAG epitope including the amino acid sequence Asp-Tyr-Lys-Asp-Asp-Asp-Asp-Lys (SEQ ID NO: 31).

62. The blocked immunoglobulin complex of embodiment 59, in which the immunoglobulin includes an antigen binding domain derived from, or forming any portion of, an antibody or antibody fragment.

63. The blocked immunoglobulin complex of embodiment 62, in which the antigen binding domain is derived from, or forms any portion of an immunoglobulin, an IgA isotype antibody, an IgD isotype antibody, an IgE isotype antibody, an IgG isotype antibody, an IgM isotype antibody, a monospecific antibody, a bispecific antibody, a Fab fragment, a Fab′ fragment, an F(ab′)₂ fragment, an Fv fragment, a rigG fragment, a scFv fragment, a scFV-Fc fragment, and a minibody fragment.

64. A blocked immunoglobulin complex, including: a heavy chain including SEQ ID NO: 42; and a light chain including SEQ ID NO: 43 and crosslinked to the blocking construct of any of embodiment 31-56 or 58.

65. A pharmaceutical composition including the blocked immunoglobulin complex of any one of embodiments 59-64.

66. The pharmaceutical composition of embodiment 65, further including a pharmaceutical excipient.

67. A method of treating cancer, including: administering a therapeutically effective amount of the blocked immunoglobulin complex of embodiment 59 to a subject in need thereof.

68. The method of embodiment 67, in which the immunoglobulin of the blocked immunoglobulin complex is cetuximab and thereby forms a cetuximab blocked immunoglobulin complex.

69. The method of embodiment 67, in which the method is a treatment of head and neck cancer in a subject including a first line treatment, a second line treatment, a locoregional head and neck cancer, and a metastatic head and neck cancer.

70. The method of embodiment 67, in which the method includes at least one of: the treatment of head and neck cancers associated with high expression of EGFR, including hypopharyngeal cancer, laryngeal cancer, lip and oral cavity cancer, metastatic squamous neck cancer, nasopharyngeal cancer, oropharyngeal cancer, paranasal sinus and nasal cavity cancer, and salivary gland cancer; or treatment of squamous cell carcinoma of the head and neck in a subject, the method including administering to the subject in need thereof a pharmaceutically or therapeutically useful amount of the blocked immunoglobin complex; or treatment of squamous cell carcinoma of the head and neck in a subject, the method including administering to the subject in need thereof: a pharmaceutically or therapeutically useful amount of the cetuximab blocked immunoglobulin complex; and a pharmaceutically or therapeutically useful amount of monalizumab; or treatment of squamous cell carcinoma of the head and neck in a subject, the method including administering to the subject in need thereof: a pharmaceutically or therapeutically useful amount of the cetuximab blocked immunoglobulin complex; and a pharmaceutically or therapeutically useful amount of ficlatuzumab; or treatment of squamous cell carcinoma of the head and neck in a subject, the method including administering to the subject in need thereof: a pharmaceutically or therapeutically useful amount of the cetuximab blocked immunoglobulin complex; and a pharmaceutically or therapeutically useful amount of palbociclib, or a pharmaceutically acceptable salt thereof; or treatment of squamous cell carcinoma of the head and neck in a subject, the method including administering to the subject in need thereof: a pharmaceutically or therapeutically useful amount of the cetuximab blocked immunoglobulin complex; and a pharmaceutically or therapeutically useful amount of cabozantinib, or a pharmaceutically acceptable salt thereof; or squamous cell carcinoma of the head and neck in a subject, the method including administering to the subject in need thereof: a pharmaceutically or therapeutically useful amount of the cetuximab blocked immunoglobulin complex; and a pharmaceutically or therapeutically useful amount of penpulimab; or treatment of squamous cell carcinoma of the head and neck in a subject, the method including administering to the subject in need thereof: a pharmaceutically or therapeutically useful amount of the cetuximab blocked immunoglobulin complex; and a pharmaceutically or therapeutically useful amount of pembrolizumab; or treatment of squamous cell carcinoma of the head and neck in a subject, the method including administering to the subject in need thereof: a pharmaceutically or therapeutically useful amount of the cetuximab blocked immunoglobulin complex; and a pharmaceutically or therapeutically useful amount of 5-Fluorouracil (5-FU); and a pharmaceutically or therapeutically useful amount of an agent selected from the group of cisplatin and carboplatin, or a pharmaceutically acceptable salt thereof; or treatment of squamous cell carcinoma of the head and neck in a subject, the method including administering to the subject in need thereof: a pharmaceutically or therapeutically useful amount of the cetuximab blocked immunoglobulin complex; and a pharmaceutically or therapeutically useful amount of paclitaxel, or a pharmaceutically acceptable salt thereof; and a pharmaceutically or therapeutically useful amount of carboplatin, or a pharmaceutically acceptable salt thereof.

71. The method of embodiment 67, in which the method includes treatment of colon cancer, including metastatic colorectal cancer, in which the cancer cells express epidermal growth factor receptor (EGFR) protein, in a subject, the method including administering to the subject in need thereof a pharmaceutically or therapeutically useful amount of the blocked immunoglobulin complex.

72. The method of embodiment 71, in which the method of treatment includes: RAS wild-type (WT) metastatic colorectal cancer in a subject in need thereof, the method including administering to the subject a pharmaceutically or therapeutically useful amount of the blocked immunoglobulin complex; or RAS wild-type (WT) metastatic colorectal cancer in a subject in need thereof, the method including administering to the subject a pharmaceutically or therapeutically useful amount of each of: the blocked immunoglobulin complex; and one or more chemotherapeutic agents selected from the group of oxaliplatin, irinotecan, regorafenib, trifluridin tipiracil (TAS-102), pembrolizumab, afatinib, tepotinib, leucovorin, 5-fluorouracil, capecitabine, bevacizumab, ziv-aflibercept, ramucirumab, panitumumab, leucovorin, and Trifluridine and tipiracil; or treatment of metastatic colorectal cancer in a subject in need thereof, the method including administering to the subject a pharmaceutically or therapeutically useful amount of each of: the blocked immunoglobulin complex; and one or more anticancer agents selected from the group of leucovorin, 5-FU, and oxaliplatin, or a pharmaceutically acceptable salt thereof; or treatment of metastatic colorectal cancer in a subject in need thereof, the method including administering to the subject a pharmaceutically or therapeutically useful amount of each of: the blocked immunoglobulin complex; and one or more anticancer agents selected from the group of leucovorin, 5-FU, and irinotecan, or a pharmaceutically acceptable salt thereof; or treatment of metastatic colorectal cancer in a subject in need thereof, the method including administering to the subject a pharmaceutically or therapeutically useful amount of each of: the blocked immunoglobulin complex; and one or more anticancer agents selected from the group of capecitabine and oxaliplatin, or a pharmaceutically acceptable salt thereof; or treatment of metastatic colorectal cancer in a subject in need thereof, the method including administering to the subject a pharmaceutically or therapeutically useful amount of each of: the blocked immunoglobulin complex; and one or more anticancer agents selected from the group of leucovorin, 5-FU, oxaliplatin, and irinotecan, or a pharmaceutically acceptable salt thereof; or treatment of RAS wild-type (WT) metastatic colorectal cancer in a subject in need thereof, the method including administering to the subject a pharmaceutically or therapeutically useful amount of each of: the blocked immunoglobulin complex; and afatinib, or a pharmaceutically acceptable salt thereof; or treatment of RAS wild-type (WT) metastatic colorectal cancer in a subject in need thereof, the method including administering to the subject a pharmaceutically or therapeutically useful amount of each of: the blocked immunoglobulin complex; and tefotinib, or a pharmaceutically acceptable salt thereof; or treatment of colorectal cancer in a subject, the method including administering to the subject in need thereof a pharmaceutically or therapeutically useful amount of each of: the blocked immunoglobulin complex, as described herein; and encorafenib, or a pharmaceutically acceptable salt thereof; or treatment of colorectal cancer in a subject in need thereof, the method including administering to the subject a pharmaceutically or therapeutically useful amount of each of: the blocked immunoglobulin complex; encorafenib, or a pharmaceutically acceptable salt thereof; and binimetinib, or a pharmaceutically acceptable salt thereof; or treatment of RAS wild-type (WT) metastatic colorectal cancer in a subject in need thereof, the method including administering to the subject a pharmaceutically or therapeutically useful amount of each of: the blocked immunoglobulin complex; vemurafenib, or a pharmaceutically acceptable salt thereof; and camrelizumab; or treatment of metastatic colorectal adenocarcinoma with mutant APC, mutant TP53 and mutant KRAS genes in a subject, the method including administering to the subject in need thereof a pharmaceutically or therapeutically useful amount of the blocked immunoglobulin complex.

73. The method embodiment 67, including use of the blocked immunoglobulin complex in a method of treatment of colon cancer, including metastatic colorectal cancer, in which the cancer cells contain at least one gene mutation selected from the group consisting of: a K-RAS (RAS) gene mutation, a RAF gene mutation, and a PI3K gene mutation in a subject, the method including administering to the subject in need thereof a pharmaceutically or therapeutically useful amount of the blocked immunoglobulin complexes.

74. The method of embodiment 73, in which one or more of: the K-RAS mutations include G12A, G12C, G12D, G12R, G12S, G12V, G13A, G13C, G13D, G13S, G13V, A146P, A146T, A146V, Q61H, Q61L, Q61R, and K117N mutations; or the method includes treating colon cancer with a K-RAS mutation present, including metastatic colon cancer with a K-RAS mutation present, in a subject, the method including administering to the subject a pharmaceutically or therapeutically effective amount of each of: the blocked immunoglobulin complex, and panitumumab; or the blocked immunoglobulin complex is used in a method of treatment of colon cancer, including metastatic colorectal cancer, in which the cancer cells overexpress EGFR ligand, in a subject, the method including administering to the subject in need thereof a pharmaceutically or therapeutically useful amount of the blocked immunoglobulin complex; or method includes treatment of metastatic colorectal cancer in a subject, the method including administering to the subject in need thereof a pharmaceutically or therapeutically useful amount of each of: the blocked immunoglobulin complex; irinotecan, or a pharmaceutically acceptable salt thereof; oxaliplatin, or a pharmaceutically acceptable salt thereof; and 5-fluorouracil, or a pharmaceutically acceptable salt thereof.

75. A method of modifying the binding activity of antigen binding domain including: providing a set of one or more blocking constructs of any of embodiments 1, 31-56, or 58; and crosslinking the set of one or more blocking constructs to an antigen binding domain to thereby modify the binding activity of the antigen binding domain.

76. The method of embodiment 75, further including exposing the set of one or more blocking constructs to an ultraviolet light trigger to activate the cleavable linker of the blocking construct to disassociate the blocking moiety from the antigen binding domain and reduce the effective concentration of block moiety at the antigen binding domain to thereby modify the binding activity antigen binding domain to an antigen.

77. The method of embodiment 76, in which the ultraviolet light trigger has an activation wavelength of 365 nm.

78. A method for producing a kappa light chain-binding polypeptide, including: expressing a nucleic acid sequence encoding the kappa light chain-binding polypeptide amino acid sequence of the kappa light chain-binding polypeptide of any of embodiments 3-30 or 57 in transformant cells to produce the kappa light chain-binding polypeptide; and extracting and purifying the produced kappa light chain-binding polypeptide from the transformant cells.

79. A method for producing a blocking construct, including: expressing a nucleic acid sequence encoding the amino acid sequence of the blocking construct of any of embodiments 1, 31-56, or 58 in transformant cells to produce the blocking construct; and extracting and purifying the produced blocking construct from the transformant cells.

80. The method of embodiment 79, in which one or more of: the transformant cells include Escherichia coli (E. coli) bacteria; the transformant cells include BL21 (DE3) strain E. coli bacteria; further including: growing the transformant cells in lysogeny broth (LB) for 12 hours or more at 37° C.; and diluting the LB 100 fold; the nucleic acid sequence is a codon optimized amino acid sequence optimized for expression in (E. coli) bacteria; the amino acid sequence is codon optimized by: amplifying both a vector and an insert with PCR primers containing compatible 5′ overhangs; and assembling the vector and the insert via a NEB HiFi assembly reaction; the nucleic acid sequence is introduced into the cells for expression by a vector; or the nucleic acid sequence is introduced into the cells using a pET21b(+) expression vector.

81. A method for producing a blocked immunoglobulin complex, including: expressing a nucleic acid sequence encoding the amino acid sequence of the immunoglobulin of the blocked immunoglobulin complex of any of embodiments 59-64 in transformant cells to produce the immunoglobulin; expressing a nucleic acid sequence encoding the amino acid sequence of the blocking construct of any of embodiments 1, 2, 31-56, or 58 in the transformant cells to produce the blocking construct; extracting and purifying the immunoglobulin and the blocking construct from the transformant cells; and exposing the immunoglobulin and blocking constructs to a crosslinker trigger to crosslink the immunoglobulin to the blocking constructs and thereby produce blocked immunoglobulin complex.

82. The method of embodiment 81, in which one or more of: the transformant cells include Escherichia coli (E. coli) bacteria; the transformant cells include BL21 (DE3) strain E. coli bacteria; further including: growing the transformant cells in lysogeny broth (LB) for 12 hours or more at 37° C.; and diluting the LB 100 fold; the nucleic acid sequence is a codon optimized amino acid sequence optimized for expression in (E. coli) bacteria; the amino acid sequence is codon optimized by: amplifying both a vector and an insert with PCR primers containing compatible 5′ overhangs; and assembling the vector and the insert via a NEB HiFi assembly reaction; the nucleic acid sequence is introduced into the cells for expression by a vector; or the nucleic acid sequence is introduced into the cells using a pET21b(+) expression vector.

83. A method for researching the binding activity of an immunoglobulin, including: selecting a immunoglobulin; crosslinking to the immunoglobulin a blocking construct selected from the blocking construct of any of embodiments 1, 2, 31-56, or 58; and measuring the binding activity of the immunoglobulin.

84. The method of embodiment 81, further including exposing the blocking construct to a trigger to activate its cleavable linker and thereby modulate the binding activity of the immunoglobulin.

85. A kit for use in modifying the binding activity of an antigen binding domain, including two or more components selected from: a kappa light chain-binding polypeptide of any of embodiments 3-30 or 57; a blocking construct of any of embodiments 1, 2, 31-56, or 58; a blocked immunoglobulin complex of any of embodiments 59-64; and a pharmaceutical composition of embodiment 65 or embodiment 66.

86. The kit of embodiment 85, further including instructions for combining the components.

### Examples—Introduction

Described herein is the demonstration that an antibody blocking strategy can be accomplished using site-specific conjugation methods without the re-expression of an antibody. In their natural form, antibodies have a native binding affinity or “on-state” in which they are capable of binding to their targets. This leads to challenges with undesirable interactions in a range of therapeutic, analytical, and synthetic applications. Modulating the binding kinetics of antibodies to turn them from an “off-state” to an “on-state” with temporal and spatial control can address many of these challenges. As disclosed herein, a method was demonstrated that blocked the antigen binding sites of antibodies in a predictable and reproducible way while maintaining the ability to use different types of triggers to restore normal binding activity. This was accomplished through the design of blocking constructs that used both covalent and non-covalent interactions with the native antibody. The blocking constructs included a Protein L-derived kappa light chain-binding polypeptide operatively connected to a flexible linker ending in a blocking moiety designed to interact with the antigen binding site of the antibody.

As disclosed herein, engineered kappa light chain-binding domains derived from Protein L were developed to enable photo-initiated crosslinking to the kappa light chain of an antibody, forming a covalent bond at a specific location on the kappa light chain of the antibody. It was observed that the formation of the covalent bond between the kappa light chain and an engineered kappa light chain-binding polypeptide including the Protein L amino acid sequence set forth in either SEQ ID NO: 1 or SEQ ID NO: 12 did not interfere with the antigen binding site of both a cetuximab antibody and an anti-FLAG antibody and did not require genetic modification of the antibodies themselves. As disclosed herein, the covalent bond approach facilitated successfully anchoring blocking constructs including flexible tethers and blocking moieties to both the cetuximab and anti-FLAG antibodies, keeping the blocking moiety in proximity to the antigen binding site of the antibodies. It was observed that such anchoring of the blocking moieties effectively created an artificially high concentration (i.e., established an effective concentration) of the blocking moieties in proximity to the binding sites of the antibodies. Thus, it was observed that an antibody can have a lower binding affinity for the blocking moiety than for the intended target and still be placed into an “off-state” because the blocking moiety is tethered in proximity to the antigen binding site of the antibody so it can outcompete the intended target. It was demonstrated that protease-cleavable and photo-cleavable cleavable linkers included in either in the blocking moiety or the flexible tether enabled controlled cutting of the cleavable linker allowing the blocking moiety to float away (i.e., dissociate) by Brownian motion after unbinding from the antibody. It was shown that cutting the cleavable linker can trigger anti-FLAG and cetuximab antibody activation to the “on-state”.

Skilled persons will understand that Protein L binding domains can bind to the kappa light chain of the antigen binding domains of a range of antibodies. Thus, the kappa light chain-binding polypeptides, blocking construct, and blocked immunoglobulin complex compositions and related methods disclosed herein are useful for therapeutic treatments and research applications related to antibodies including a kappa light chain. Thus, antibodies that may be selectively activated or deactivated, (i.e., modulated between their “on” and “off” state) are useful for reducing the side effects of cancer immunotherapy by localizing the native binding activity of the therapy to where it is needed.

The noncovalent blocking of the antigen binding site was achieved using blocking moieties designed specifically for the antibodies. The blocking moieties had a relatively low binding constant when in free form, but had an artificially elevated binding constants when operatively connected via the flexible tether to kappa light chain binding polypeptide covalently bound to the kappa light chain of the antibody. The covalent bond kept the blocking moiety in proximity to the antigen binding site by intramolecular interaction, encouraging rebinding after the natural unbinding of the blocking moiety. The proximity of tethered blocking moieties to the antigen binding sites of the antibodies created an effectively high local concentration of the blocking moieties at the antigen binding sites, allowing blocking moieties to successfully outcompete the intended antibody targets and keep the antibodies in an “off-state.”

The flexible tether was designed to be cleaved, and, once cleaved, to release the blocking moiety, allow it to naturally unbind and move out of proximity of the antibody away due to Brownian motion, preventing it from rebinding. This effectively converts the antibody to an “on-state”. In this way, stabile long-term blocking of antigen binding sites along with the capability of quickly restoring the native binding activity of an antibody upon activation without making any changes to the structure of the native antibody itself was achieved. The flexible tether designs and embodiments included herein allow for tethers with including both proteinaceous and non-proteinaceous linkers, the linkers configured to cleave and activate the antibody in response to light, and thereby create photoactivated therapeutic antibodies. Enzymatic cleavage may also be employed as the activating trigger as well, which enables flexibility of the activation mechanism for specific applications.

The covalent binding of a flexible tether molecule to the antibody was achieved using site-specific conjugation methods.

At least some of the subject matter discussed herein and/or in the following Examples was published on Dec. 10, 2022, as Brasino et al., Comm. Biol. 5, Art. 1357, 2022 (doi.org/10.1038/s42003-022-04094-1).

### Example 1: Designing a Tetherable Blocking Moiety Using Protein L

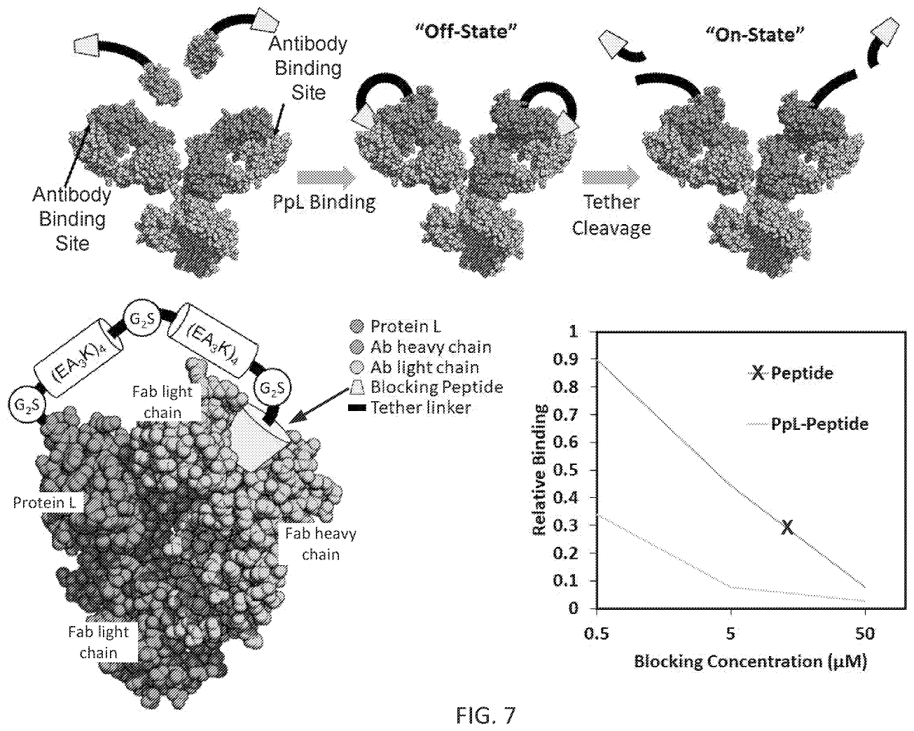

FIGS. 7A, 7B, 8 and 9 demonstrate the attachment of a blocking construct including a tethered blocking moiety to a FLAG antibody with successful inactivation and subsequent photoactivation of the FLAG antibody.

FIG. 7A shows a schematic of an antibody activation strategy. Currently, antibody blocking techniques do not exist that can simultaneously create predictable long-term blocking of the antigen binding site and also allow for quick spatially controlled unblocking and activation of the antibody. Here, the challenge of pairing controlled blockade and spatially specific activation of the antibody was addressed through the design of a tether-based blocking construct that takes advantage of simultaneous covalent and noncovalent attachments to the antibody as shown in FIG. 7A. The noncovalent blocking of the antigen binding site allows for selective and quick activation after the flexible tether is cleaved from the covalently bound blocking construct as described herein.

To keep the blocking moiety in proximity to an antibody's binding pocket, a flexible tether needed to be bound to the antibody itself. To site-specifically attach the flexible tether to the antibody, a single B domain of Protein L (PpL) was used, due to its documented site-specific binding to kappa light chain which are found a majority of human (and mouse) antibodies. Protein L from the Peptostreptococcus magnus (a.k.a., Finegoldia magna) bacteria contains several repeated B domains and C domains which are kappa light chain-binding domains that bind to subtypes of the kappa light chain, without interfering with antigen recognition. Moreover, unlike similar antibody binding proteins, PpL has no affinity for the antibody fragment crystallizable (Fc) region, which mediates the function of immunotherapeutic antibodies.

FIG. 7B is a graphical rendering of the crystal structure (PDB 1MHH) of Protein L (PpL) bound to a Fab fragment of an IgG isotype antibody and shows a graphical representation of a flexible linker having from N-terminus to C-Terminus a structure represented by the polypeptide formula: (G₂S)-(EA₃K)₄-(G₂S)-(EA₂K)₄-(G₂S) (SEQ ID NO: 37). To design a flexible tether, the distance between the C-terminus of the PpL protein and the binding pocket of the antibody was estimated to be 7 nm as shown in FIG. 7B. It was reasoned that a tether needed to be a flexible tether in order to reach over the lip of the antigen binding sites (i.e., antigen binding pocket) of many antibodies. To satisfy both these constraints, a flexible tether including a synthetic linker composed of synthetic alpha helices for appropriate length was created (Li et al., Appl. Microbiol. Biotechnol. 100:215-225, 2016), separated by short stretches of glycine-serine sequences for flexibility as shown in FIG. 7B.

As shown in FIG. 7B, the path of a flexible linker is shown in cartoon form with the different segments labeled with their sequence. The flexible linker shown is attached to the C-terminus of PpL and reaches up and across the antigen binding pocket between the light and heavy chains of an antigen binding domain. In order to bend, the flexible linker shown in FIG. 7B was broken into two alpha helical segments made to be 3 nm each.

FIG. 7C is a line graph showing that a FLAG blocking moiety linked to a Protein L (PpL) kappa light chain binding-domain blocks an anti-FLAG antibody better than use of a FLAG blocking moiety alone. The relative binding was gauged through ELISA and is expressed in relation to anti-FLAG antibody alone.

### Example 2: Covalent Attachment of a Flexible Tether to an Antibody Through Use of a Photo-Reactive Crosslinkable Protein L Kappa Light Chain-Binding Domain

Covalent attachment of a PpL kappa light chain-binding domains to an antibody was accomplished by modifying the PpL kappa light chain-binding domains to contain a non-canonical amino acid with a reactive side chain. Using an amber codon suppression technique known in the art (Chin et al., PNAS 99:11020-11024, 2002), the photo-crosslinker non-canonical amino acid 4-benzoyl phenylalanine (BpA) was substituted at various positions within a predicted binding interface between the PpL kappa light chain-binding domains and the kappa light chain to determine which site resulted in the best binding efficiency. Skilled persons will understand that BpA or other photo-reactive non-canonical amino acid residues may be used to create site specific covalent attachments (i.e., crosslinking) between proteins including proteins G and A to IgG (Kanje et al., Bioconjugate Chem. 27:2095-2102, 2016; Perols & Karlström, Bioconjugate Chem. 25:481-488, 2014). However, the use of photo-reactive non-canonical amino acid residues with PpL has not been demonstrated previously.

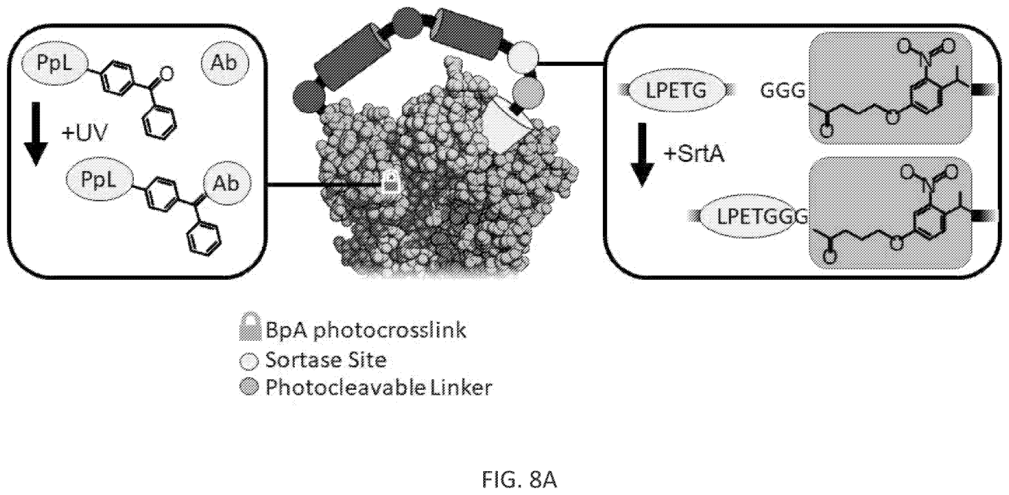

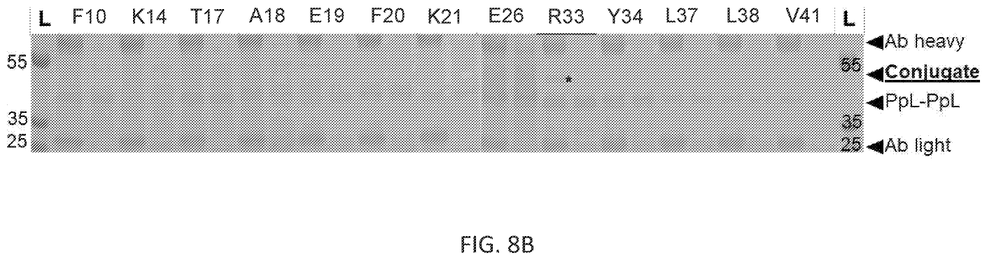

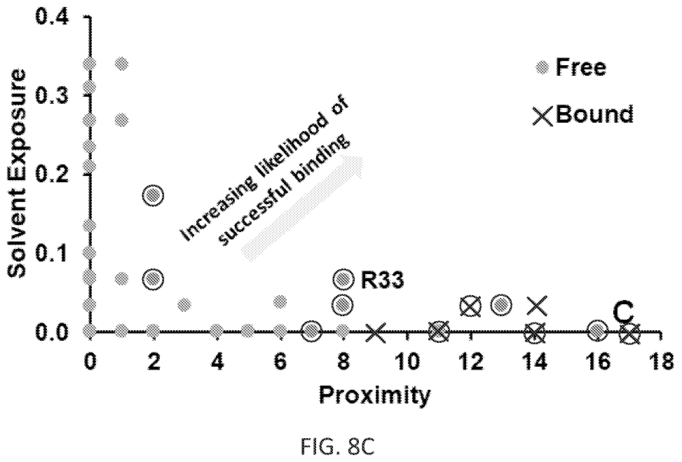

FIG. 8B is an image of a reducing SDS PAGE gel with 50 μM of Protein L irradiated with 1 μM mouse IgG1 kappa antibody showing different substitution positions chosen on the amino acid sequence of Protein L to introduce the photo-reactive non-canonical amino acid Benzoyl-4-Phenylalanine. Crosslinking between the kappa light chain of the IgG1 kappa antibody and PpL kappa light chain binding domains was only observed with PpL kappa light chain-binding domains including a R33Bpa substitution.

FIGS. 8A and 8B shows the successful photoconjugation of Protein L to the kappa light chain of an anti-FLAG antibody to block the anti-FLAG antibody followed by photoactivation. As shown in FIGS. 8A and 8B, different substitution positions were chosen on protein L to introduce the photo-reactive non-canonical amino acid Benzoyl-4-Phenylalanine (BpA). A reducing SDS-PAGE gel with 50 μM of each PpL mutant was irradiated with 1 μM mouse IgG1 kappa antibody showed a photo-crosslinked product between the kappa light chain and PpL at residue position with a R33BpA mutation (* marks the R33BpA band in the gel).

Initial experiments using this PpL-Linker-peptide blocking construct demonstrated that both a blocking moiety including the FLAG epitope amino acid sequence: “Asp-Tyr-Lys-Asp-Asp-Asp-Asp-Lys”(SEQ ID NO: 31), operatively connected to a crosslinker kappa light chain-binding polypeptide including the Protein L amino acid sequence set forth in SEQ ID NO: 1, worked together to block an Anti-FLAG antibody (clone 1557CT661.18.1), helping validate the blocking construct design as shown in FIG. 8B. However, it was predicted that the blocking efficiency could be further increased by covalently binding PpL to the antibody to avoid transient binding, thereby keeping the binding peptide tethered to the antibody and keeping the antibody in the “off-state” until the tether was cleaved. As shown in FIG. 8B, a PpL, covalently bound to an anti-FLAG antibody and operatively connected to a blocking moiety including a FLAG epitope, the FLAG epitope including the amino acid sequence: “Asp-Tyr-Lys-Asp-Asp-Asp-Asp-Lys” (SEQ ID NO: 31), was shown to block the anti-FLAG antibody better than the combining of anti-FLAG antibody with unconjugated blocking moiety. The relative binding affinity was gauged through ELISA and is expressed in relation anti-FLAG antibody alone.

As shown in FIG. 8B, the PpL-linker-FLAG construct with BpA was modified at various locations and each individual mutant screened for photo-crosslinking to a mouse IgG1 kappa Anti-CD3 antibody. The use of the mouse IgG1 kappa Anti-CD3 antibody, which does not bind the FLAG antigen, precluded any interference due to binding of the blocking moiety to the antibody. Of all mutants screened, only one, with BpA substituted for the R33 residue (PpL-R33BpA) showed significant photo-crosslinking to the mouse IgG1 kappa Anti-CD3 antibody.

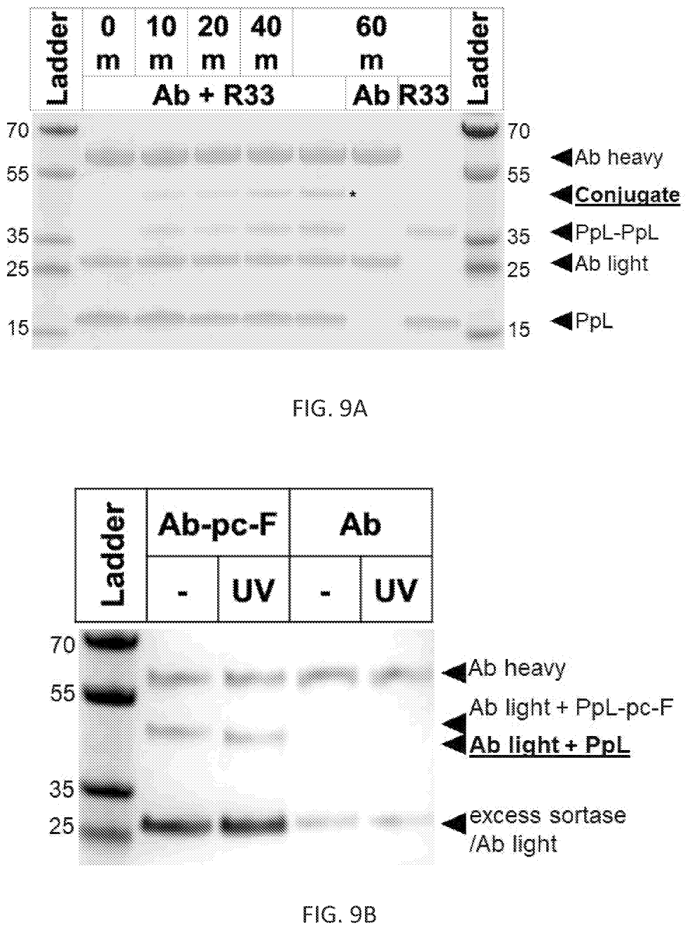

FIGS. 9A and 9B are images of reducing SDS PAGE gels showing, respectively, 100 μM of PpLR33BpA (R33) with 4 μM mouse IgG1 kappa antibody (Ab) irradiated under 360 nm light for the time indicated, and the R33 mutant fused to the flexible tether of FIG. 7B and crosslinked to an anti-FLAG antibody and then operatively connected enzymatically to a blocking moiety including a photo-cleavable linker. As shown in FIG. 9A, a reducing SDS-PAGE gel showing 100 μM of PpLR33BpA (R33) with 4 μM mouse IgG1 kappa antibody (Ab) was irradiated under 360 nm light for the time indicated.

As shown in FIG. 9B, the specificity of conjugation of the PpLR33BpA was then explored. The R33 mutant fused to the flexible tether was photoconjugated to the anti-FLAG antibody and then enzymatically attached to a photocleavable blocking moiety. Light exposure for 10 minutes led to photocleavage and loss of the blocking moiety

Photo-crosslinking occurred between the PpL construct and the IgG light chain, while no attachment to the heavy chain was observed. PpL dimers were also observed, but both dimers and un-reacted monomers were cleaned from the photoconjugated antibody through size exclusion techniques. PpL binds most subtypes of the kappa light chain and these subtypes are common in biological applications, leading to PpL binding more than half of all immunoglobulins in human serum and over a third in mice (Nilson et al., J. Immunol. Methods 164:33-40, 1993). As such, this novel photo-crosslinking mutant should allow covalent attachment to antibodies including kappa light chains used in the lab and clinic as both research tools and therapeutic compositions. In some embodiments, this conjugation strategy may facilitate the site-specific conjugation of antibody Fab fragments, for which there are currently few options known in the art.

### Example 3: Blocking and Activating an Anti-FLAG Antibody

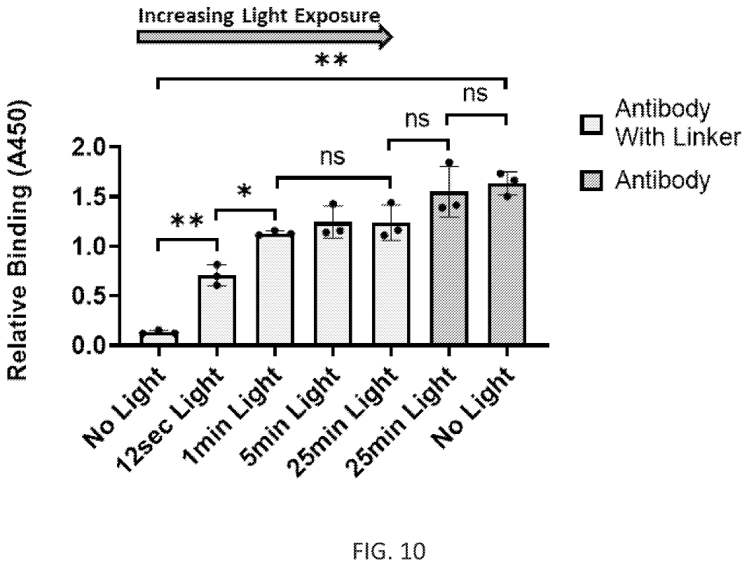

FIG. 10 is a graph showing the binding activity of anti-FLAG antibody alone modified with a blocking moiety including a photocleavable linker after photoirradiation for the indicated time (n=3). FIG. 10 shows the binding activity of anti-FLAG antibody alone modified with the photocleavable blocking moiety (as also shown in FIG. 9B), after photoirradiation for the indicated time (n=3). The tethered blocking moiety successfully reduced the binding efficiency of the anti-FLAG antibody to its target and was removed with brief irradiation, leading to light activation of the antibody binding. All gels were labeled with size control ladder bands in kDa.

The blocking construct of claim 10, in which the kappa light chain-binding polypeptide, the blocking moiety, or the flexible tether further include a conjugation moiety selected from any of the group consisting of: a sortase recognition site including the amino acid sequence Leu-Pro-Glu-Thr-Gly (SEQ ID NO: 33).

Next, as shown in FIG. 9B, it was observed that the R33 mutant could be used to modify an anti-FLAG antibody. Furthermore, reversable blocking of the anti-FLAG antibody covalently bound to a kappa light chain-binding polypeptide including an R33 substituted kappa light chain-binding domain (i.e., a R33 substituted kappa light chain-binding polypeptide) was achieved by operatively connecting a blocking moiety with an n-terminal photo-reactive cleavable linker. In this instance, to avoid cleaving the photo-reactive cleavable linker during the photo-conjugation reaction, the blocking moiety was attached only after the R33 mutant (SEQ ID NO: 1, or SEQ ID NO: 12) was operatively connected by photoconjugation to the antibody. To enable photoconjugation to the antibody, a Sortase A recognition site including the amino acid sequence: “Leu-Pro-Glu-Thr-Gly” (SEQ ID NO: 33) was included at the C-terminus and a polypeptide blocking moiety (SEQ ID NO: 31), the polypeptide blocking moiety including a photo-reactive cleavable linker and a Tri-Glycine motif at its N-terminus Sortase A enzyme was then used to create an amide bond between the C-terminus of the Sortase A recognition site and the N-terminal glycine of the synthetic peptide. Light exposure lead to the photocleavage of the blocking moiety as observed by gel shown in FIG. 9B. This corresponded to successful restoration of anti-FLAG binding abilities, while the anti-FLAG antibody itself was unaffected as shown in FIG. 10. This demonstrated the ability to temporally control the activation of the anti-FLAG antibody and enable the use of light to control the spatial activation of the antibody within a sample.

### Example 4: Enzymatic and Photo-Reactive Blocking and Activating the Therapeutic Antibody Cetuximab

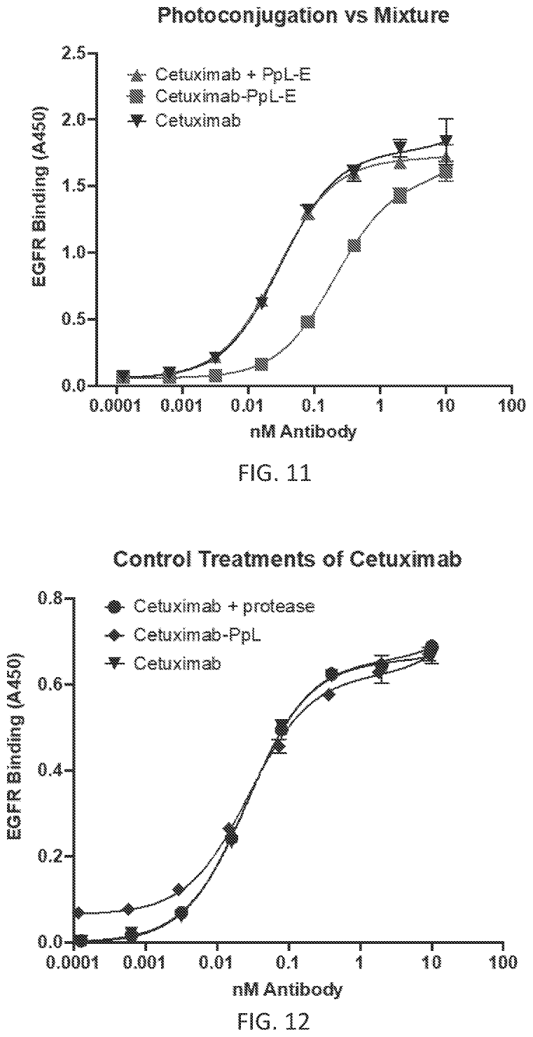

This photo-conjugation and reversable blocking strategy was then tested on the therapeutic antibody cetuximab, which binds to epidermal growth factor receptor (EGFR) and is routinely used in immunotherapy for multiple cancers (Desnoyers et al., Science Translational Medicine 5: 207ra144-207ra144, 2013). For this, a blocking moiety including the amino acid sequence: Gln-Gly-Gln-Ser-Gly-Gln-Cys-Ile-Ser-Pro-Arg-Gly-Cys-Pro-Asp-Gly-Pro-Tyr-Val-Met-Tyr (SEQ ID NO: 32), known to transiently block cetuximab was fused at the C-terminal end of a flexible tether attached to a R33-substituted kappa light chain polypeptide (SEQ ID NO: 1 or SEQ ID NO: 12), which readily photo-conjugated to the kappa light chain of the cetuximab. The resulting photo-conjugate was filtered to remove un-bound PpL and then tested for affinity to EGFR via ELISA, where it was found to bind with a significantly lower binding affinity than the un-modified cetuximab (as shown in FIG. 11).

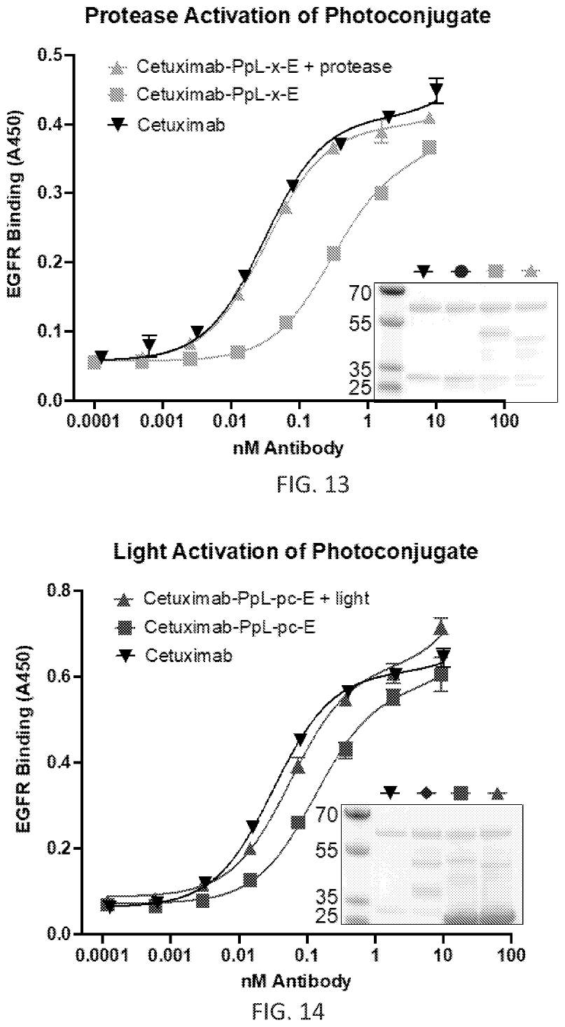

For comparison, no difference in binding affinity was observed when cetuximab was mixed with a two-fold excess of control blocking construct, which lacked the photo-crosslinking mutation. This demonstrated the utility of covalent bonding through protein L kappa light chain binding domain photo-conjugation, as the combined avidity of both the blocking moiety and unmodified protein L were insufficient to keep the construct in place and block cetuximab binding to EGFR antigen. There was also no change in affinity when a construct without a blocking moiety was photo-conjugated. As shown in FIG. 13, the covalent bonding of an engineered crosslinker PpL (SEQ ID NO: 1 or SEQ ID NO: 12) to cetuximab (even with an attached linker but no blocking moiety) did not interfere with cetuximab's native binding activity, and indicated that this modification strategy will not adversely affect the therapeutic behavior of the antibody once the flexible tether is cleaved.

FIG. 11 is a line graph comparing cetuximab affinity for EGFR after being photoconjugated to a EGFR blocking construct versus being combined with non-conjugated EGFR blocking construct. As shown in FIG. 11, cetuximab was photoconjugated with an EGFR blocking construct including a R33-substituted kappa light chain-binding polypeptide to form a blocked immunoglobulin complex (C-PpL-E) and combined with a non-conjugated EGFR blocking construct including wild type PpL kappa light chain-binding polypeptides (PpL-E). The addition of two molar excess PpL-E had no significant effect on cetuximab binding affinity suggesting that photoconjugating the R33-substituted kappa light chain-binding polypeptide to the cetuximab antibody to form a blocked immunoglobulin complex facilitates establishing an effective concentration of a blocking construct at the antigen binding site of the cetuximab antibody (Kd values-cetuximab 31 PM, C-PpL-E 201 pM, cetuximab+PpL-E 27 pM). Thus, kappa light chain-binding polypeptides including kappa light chain-binding domains having photo-reactive crosslinkers (crosslinker kappa light chain binding domains) are useful for anchoring blocking constructs to immunoglobulins to form blocked immunoglobulin complexes.

FIG. 12 is a line graph showing that a chymotrypsin treatment had no detectable effect on the binding affinity of cetuximab itself, nor did the photoconjugation of a blocking construct lacking a blocking moiety including an EGFR epitope (SEQ ID NO: 32) (C-PpL-No) (Kd values—cetuximab 26 pM, cetuximab+Digest 26 pM, C-PpL-No 28 PM) (n=3).

FIG. 13 is a line graph and an inset image, the inset image is a reducing SDS PAGE gel and ELISA (n=3) showing cetuximab alone vs. cetuximab photoconjugated to a PpL-R33BpA blocking construct (SEQ ID NO: 1 operatively connected to SEQ ID NO: 32 via SEQ ID NO: 37) including a chymotrypsin cleavable linker (C-PpL-X-E), with and without protease treatment with chymotrypsin. As shown in FIG. 13, the line graph shows that the cetuximab photoconjugated to the PpL-R33BpA blocking construct had a 9-fold lower EGFR binding affinity compared to the EGFR binding affinity of cetuximab alone. The protease treatment of this photoconjugate resulted in a decrease in the molecular weight of the photoconjugated light chain (corresponding to loss of the blocking moiety) and rescued cetuximab affinity (Kd values—C-PpL-x-E 293 pM, C-PpL-x-E+Protease 33 pM, cetuximab 31 pM).

Protease activation was then used to test if the photo-conjugate binding could be restored by removing the blocking moiety. For this, a short chymotrypsin cleavable peptide amino acid sequence (i.e., a protease cleavage site) was inserted between the linker and blocking moiety sequence to form an enzymatically cleavable blocking construct and the enzymatically cleavable blocking construct was conjugated to cetuximab. As shown in FIG. 13, chymotrypsin exposure did not affect the cetuximab binding activity. A SDS PAGE gel was used to monitor the successful photo-conjugation of the enzyme cleavable tether to cetuximab as well as cleavage of the blocking moiety from the light chain upon chymotrypsin incubation (as shown in the inset of FIG. 13). The photo-conjugation of the enzymatically cleavable blocking construct to cetuximab successfully blocked cetuximab from binding EGFR until activation with the protease chymotrypsin, at which point its affinity increased 9-fold as determined by ELISA (as shown in FIG. 14).

FIG. 14 is a line graph and inset image, the inset image is a reducing SDS PAGE gel and ELISA (n=3) of comparing the EGFR binding between cetuximab and cetuximab blocked immunoglobulin complexes configured with and without EFGR epitopes. Cetuximab was photoconjugated to a blocking construct lacking a blocking moiety having an EGFR epitope (the blocking construct including SEQ ID NO: 1 operatively connected to SEQ ID NO: 37 only) (C-PpL-No) and a blocking construct including an EGFR epitope (SEQ ID NO: 1 operatively connected to SEQ ID NO: 32 via SEQ ID NO: 37) (C-PpL-PC-E) to form, respectively, C-PpL-No and C-PpL-PC-E blocked immunoglobulin complexes. The EGFR binding of the cetuximab, C-PpL-No blocked immunoglobulin complex, and C-PpL-PC-E blocked immunoglobulin complex was measured after 10 minutes with and without light exposure. As shown in FIG. 14, the C-PpL-PC-E blocked immunoglobulin complex had a decreased affinity for EGFR and the C-PpL-No blocked immunoglobulin complex had a decreased affinity for EGFR. Light exposure of C-PpL-PC-E blocked immunoglobulin complex lead to a decrease in molecular weight of the kappa light chain (corresponding to the loss of the blocking moiety), and EGFR affinity being largely restored (Kd values-cetuximab 31 pM, C-PpL-PC-E 131 pM, C-PpL-PC-E+UV 57 pM) (n=3).) All gels were labeled with size control ladder bands in kDa.

To activate cetuximab with light, a blocking moiety was attached to a flexible tether including a 4-{4-[1-(9-Fluorenylmethyloxycarbonylamino)ethyl]-2-methoxy-5-nitrophenoxy}butanoic acid (Fmoc) photocleavable linker (CAS 162827-98-7) using the same activation strategy disclosed herein for modulating the binding activity of anti-FLAG antibody. As shown in FIG. 14, SDS PAGE gel analysis indicated the successful attachment of the blocking construct tethering the photocleavable blocking moiety to cetuximab with release of the blocking moiety upon 365 nm light exposure. Attaching the photocleavable blocking moiety (via the photoconjugated blocking construct) to cetuximab led to a 4-fold decrease in cetuximab's binding affinity to EGFR, and light exposure restored cetuximab to its native or original binding affinity. This 4-fold activation was lower than that of the protease-cleavable blocking construct due to the higher apparent binding affinity of the protease-cleavable blocking construct prior to being exposed to light.

It was observed that blocking constructs produced as fusion proteins (i.e. the kappa light chain-binding polypeptide, flexible linker, and blocking moiety being expressed a single chain of amino acids) photoconjugated more completely to cetuximab than blocking constructs produced using a sortase reaction requiring a sortase recognition site. Photo-conjugation could be enhanced by fusing a blocking moiety to the C-terminus of the Sortase site, however, this then hindered the Sortase reaction from completely substituting this bound blocking moiety for the synthetic photocleavable version. Judging by PAGE gel analysis, the majority of cetuximab light chains are conjugated with Protein L in either format. In some embodiments, a cetuximab blocked immunoglobulin complex including one light chain left un-conjugated to a blocking construct as disclosed herein may produce cetuximab with even lower affinities before activation with light or protease treatment.

### Example 5: Methods for Expressing Protein L Kappa Light Chain-Binding Polypeptides

A single monomer of the multimeric Protein L (PpL) was produced to create a precise anchoring point for the blocking constructs disclosed herein For this, an engineered C* domain was used (SEQ ID NO: 1 or SEQ ID NO: 12) which is nearly identical to the C4 domain of wildtype Protein L (Uniprot Q51918). Skilled persons will understand that the C* domain forms the same secondary structure as its wildtype counterpart and binds the kappa light chain with a dissociation constant of 130 nM (Graille et al., Structure 9:679-687, 2001).

It was reasoned that a useful blocking construct would include a flexible tether configured to have an end-to-end length to tether the blocking moiety at a sufficient movement radius for the blocking moiety to establish an intramolecular binding interaction between its epitope and the antigen binding site sequence. Accordingly, a polypeptide flexible tether including the amino acid sequence set forth in SEQ ID NO: 37, was operatively connected to the C-terminus of the crosslinker kappa light chain-binding polypeptide set forth in SEQ ID NO: 1 to form a PpL fusion protein.

For flexibility, these alpha helices were separated from the crosslinker kappa light chain-binding polypeptide and each other by short sequences of glycine and serine. The amino acid sequence of the fusion protein was then codon optimized for expression in E. coli and synthesized (Integrated DNA Technologies) before being inserted into the pET21b(+) expression vector (EMD Millipore). This was done by amplifying both vector and insert with PCR primers containing compatible 5′ overhangs and then assembling them via the NEB HiFi assembly reaction (New England Biolabs). Proper insertion was confirmed via sanger sequencing (Genewiz). For expression, the BL21 (DE3) strain of E. coli (ThermoFisher) was transformed with this plasmid and maintained in 100 μg/ml ampicillin (GoldBio) for selection. Transformants were grown in 5 ml of LB overnight at 37° C., followed by a 100-fold dilution into LB the following morning. Once this new culture reached mid-log growth, as indicated by an OD₆₀₀ of 0.4, IPTG was added to a final concentration of 1 mM to induce the expression the PpL fusion protein. Cultures were allowed to express for 4 hours followed by centrifugation at 10,000 g to collect cells and remove culture media. Cells were then lysed by freezing pellets overnight at −20° C. followed by resuspension in 30 ml of equilibration buffer (20 mM Phosphate Buffer at pH 7.6, 300 mM NaCl, 10 mM Imidazole), followed by sonication using a probe sonicator (Qsonica, model Q500) with half inch probe diameter for 4 min total with 30 min on/off pulses, 40% amplitude. Insoluble material was then removed through centrifugation at 12,000 g for 20 min. The PpL fusion proteins expressed on pET vectors contained a c-terminal 6×His tag and were purified using immobilized metal affinity chromatography. 50 μL of sedimented Ni-NTA coated agarose beads (ThermoFisher) were added to the soluble fraction and allowed to bind for 1 hour at 4° C. in an end over end mixer. Beads were then removed via centrifugation at 700 g for 2 min and then washed four times with 400 μL wash buffer (20 mM PB, 300 mM NaCl, 25 mM Imidazole). Finally, the PpL fusion protein was eluted from the beads in 200 μL elution buffer (20 mM PB, 300 mM NaCl, 250 mM Imidazole). The eluate was then transferred into PBS using 7 kDa MWCO desalting columns (ThermoFisher) and quantified via A280 signal with the predicted extinction coefficient of the eluted PpL fusion proteins.

### Example 6: Mutagenesis of Protein L for Photo-Crosslinking

To further modify PpL to covalently attach to the kappa light chain, the non-canonical amino acid p-Benzoyl Phenylalanine (BpA) was substituted at multiple locations in and around the previously determined binding interface (Graille et al., J. Biol. Chem. 277:47500-47506, 2002). Fourteen amino acids were initially chosen for substitution. Using PCR mutagenesis (Q5 site-directed mutagenesis kit, New England Biolabs), the codon for each amino acid was mutated to the amber stop codon (TAG) to allow for BpA incorporation via the amber suppression method (Young et al., J. Mol. Biol. 395:361-374, 2010). pET vectors containing the mutated PpL proteins were then co-transformed into the BL21 (DE3) E. coli strain along with the pEVOL-pBpF plasmid (provided by the lab of Peter G. Schultz) which contains both the aaRS and tRNA needed to incorporate BpA at amber codons. The resulting transformants were grown under selection with 100 μg/ml ampicillin and 25 μg/ml chloramphenicol. For expression, transformants were grown overnight in 5 ml of LB at 37° C. followed by 1:100 dilution the following morning. This production culture was typically as little as 50 ml but could be scaled up as necessary. Cultures were grown until mid-log phase (OD₆₀₀=0.4) at which point IPTG was added to 1 mM final to induce mutant PpL expression and arabinose was added to 0.2% (wt/vol) final to induce aaRS and tRNA expression from pEVOL. At the same time BpA was added directly to the cultures for a final concentration of 1 mM. Cultures were allowed to express for 4 hours followed by centrifugation at 10,000 g to collect cells and remove culture media. Purification of mutant PpL was performed in the same manner as PpL detailed above.

Photo-Crosslinking—In initial screens, PpL mutants and antibodies were diluted into PBS pH7.6 such that the final concentrations were 50 μM and 2 μM respectively and loaded into thin walled 200 μL polypropylene microtubes (PCR tubes). This mixture was then irradiated for 1 hour under near UV (e.g., 365 nm) light at an intensity of 6.4 mW/cm² from an LED source (M365LP1, Thor Labs) at a distance of 14 cm. Products were reduced using dithiothreitol (DTT) solution (available commercially from ThermoFisher Scientific; Cat. No. R0861) and separated on 4-12% BisTris PAGE gels (ThermoFisher) to observe photo-crosslinking. Photocleavage was accomplished using the same irradiation setup.

Measuring Relative Binding Affinity—The ELISA's were performed using NeutrAvidin coated plates with SuperBlock Blocking Buffer (ThermoFisher, Cat. No: 15127). Each incubation step was allowed to proceed for 1 hour at room temperature with shaking at 300 rpm. Between each incubation step the ELISA was washed by hand via Multichannel with 200 μL of a Tris-buffered saline/tween solution (TBST) three times. (Skilled persons will understand that in molecular biology arts polysorbate 20 surfactant is known as “Tween.”) The plate was stamped out after washing to remove any remaining TBST before loading the next reagent. Two columns for each combination to be tested (Cetuximab; unconjugated cetuximab and blocking construct; and Cetuximab blocked immunoglobulin complex) had 100 μL of 0.1 nM Biotinylated EGFR (Acro Biosystems #EGR-H82E3) in TBST+3% BSA loaded into each well. One column for each Ab to be tested was loaded with 100 μL of just TBST+3% BSA as a non-specific binding control. The plate was then allowed to incubate. A concentration curve of each combination was prepared in TBST+3% BSA. The curves had a starting concentration of 10 nM and were serial diluted 1:5. The plate was then washed, and 100 μL of each point of each curve were loaded into their three respective columns and allowed to incubate. The plate was washed and then 100 μL of Protein G-HRP (Invitrogen #101223) diluted 1:5000 in TBST+3% BSA was loaded into each well and allowed to incubate. The plate was washed again and 100 μL of 1-Step Ultra TMB (Thermo #34029) was loaded into each well. After 10 minutes the reaction was quenched by adding 100 μL of 1M H₂SO₄ to each well, and the absorbance at 450 nm was read using a Tecan Spark 20M plate reader.

Sortase-mediated attachment of blocking moieties—Sortase A was expressed in E. coli using plasmid pET28a-SrtAdelta59 (Addgene #51138; Guimaraes et al., Nat Protoc 8:1787-1799, 2013), and purified using Ni-NTA coated agarose beads (ThermoFisher). A photocleavable blocking moiety was synthesized (Biopeptide Inc.) with three N-terminal glycine residues followed by a photocleavable linker (Santa Cruz Biotechnology) and an EGFR blocking sequence. Cetuximab crosslinked to PpL fusion protein was diluted to 1 μM in TBS with 10 mM CaCl₂) along with 50 μM purified Sortase and 200 μM of the synthesized blocking moiety to operatively connect the synthesized blocking moieties to the PpL fusion proteins by sortase reaction.

### Example 7: Blocking and Activating Antibodies Using Light for Therapeutic Compositions and Methods

Here, the successful blocking and unblocking of the anti-FLAG antibody and cetuximab antibodies demonstrated that the compositions and methods disclosed herein are useful as both research tools and therapeutic treatments. It was demonstrated that using Protein L derived kappa light chain-binding domains engineered as crosslinkers provided a consistent anchor mechanism for tethering a blocking moiety to an immunoglobulin and establishing an effective concentration of the blocking moiety at the antigen binding site of the immunoglobulin. Tethering the blocking moiety near the binding site of the antibody allowed the blocking moiety to outcompete the intended target thereby putting the antibodies into an “off-state.”

Moreover, both enzymatic and photocleavage of embodiments of the cleavable linker resulted in restoration of the binding capabilities of the antibodies. This flexibility in activation mechanisms increases the types of applications this technique can be used for. For example, in some embodiments, a tumor-directed blocked immunoglobulin complex may include a blocking construct in which the cleavable linker is a protease cleavage site configured to cleave upon its activation by a tumor-related protease. Whereby, when in the proximity of a tumor expressing the tumor-related protease, the protease cleavage site is activated by the tumor-related protease enzyme, cleaving the blocking moiety from the blocking construct and thereby selectively restoring the native binding activity of the immunoglobulin. Skilled persons will understand that the ability to selectively activate an immunoglobulin in the proximity of a tumor is useful for enhancing the specificity of the immunoglobulin-tumor binding interaction.

The use of light as an activating trigger is particularly useful for therapeutic applications. It allows for the use of photocleavable linkers that are resistant to enzymatic based cleavage making light exposure the main source of antibody activation. This addresses the challenges of using biochemical or enzymatic based triggers that are overexpressed in tumors (Breistøl et al., Eur J Cancer 34:1602-1606, 1998; Gopin et al., Bioconjugate Chem. 17:1432-1440, 2006; Gopin et al., Bioconjugate Chem. 17:1432-1440, 2006) but also present in non-target tissues (Park, et al., Clin Cancer Res 8:1172-1181, 2002; Park, et al., Clin Cancer Res 8:1172-1181, 2002), especially in the liver (Ibsen et al., Pharm Res 27:1848-1860, 2010; Miwa et al., Eur J Cancer 34:1274-1281, 1998), which could result in large scale non-localized activation of the antibody. It also allows for the activation of the antibody in tissue regions that do not have significant overexpression of tumor related proteases, such as in the tumor draining lymph nodes, where immunotherapy is the most effective (Francis et al., Sci Transl Med. 12, eaay3575, 2020; Kwon et al., Theranostics 9:8332-8343, 2019; Murthy et al., J Natl Cancer Inst. 109(12): djx097, 2017). Light activation can also be applied to a larger population of patients because it is independent of specific tumor based biochemistry which can have high variability between cancer patients (Fradet et al., PNAS 84:7227-7231, 1987).

The wavelength of light is critical to achieve spatial localization within the body. Here 365 nm light was chosen, which has been shown to be effective at triggering photocleavage of our construct and has low absorption by internal tissue (Yang et al., J Clin Laser Med Surg 19:35-39, 2001) as well as DNA (Sutherland et al., Radiat Res 86:399-409, 1981) reducing possible tissue damage from light exposure. This wavelength has sufficient penetration depth in internal tissue to activate photocleavable prodrugs at the periphery of a 1 cm diameter tumor when delivered to the tumor's center (Ibsen et al., Photochem Photobiol 89:698-708, 2013). The 365 nm light is highly scattered by the tissue (Van Staveren et al., Appl. Opt., 30:4507-4514, 1991) which helps to create a more uniform exposure of the tissue region of interest from a single point of delivery (Ibsen et al., Photochem Photobiol 89:698-708, 2013). One of the benefits of 365 nm light is that although it has significant penetration depth through internal tissues it does not penetrate deeply through skin where the intensity is reduced by 99% in the first 1 mm due to melanin absorption (Elisseeff et al., PNAS 96:3104-3107, 1999). This lack of pentation through skin prevents uncontrolled activation of the antibody from external light sources helping to localize activation to just the internal tissue region where the light has been purposely delivered. In some embodiments, the 365 nm light can be delivered through the skin to the tissue region of interest by fiber optic coupled light emitting diode systems (Ibsen et al., Photochem Photobiol 89:698-708, 2013) or through miniaturized light emitting diode technology where elements can be made with submillimeter dimensions (Wilson, Proceedings of the 22nd Annual ACM Symposium on User Interface Software and Technology, ACM, 2009) allowing them to be implanted using biopsy needles.

For Cetuximab, the blocking and activating effect was observed to be smaller with the photocleavable technique compared to the enzymatic technique. This may be caused by the synthesis process used to attach the flexible tether. The activation wavelength of the photo-reactive group in protein L was the same wavelength that could trigger cleaving the flexible tether, making the attachment a two-step process reducing overall yield and causing blocked and unblocked antibodies to be present in the sample. This was not a problem for the enzymatic cleavable blocking construct because it could be synthesized in a single photo crosslinking step.

Here, the successful blocking and controlled unblocking of antibodies was demonstrated for the first time using a blocking construct that takes advantage of both precise covalent and noncovalent interactions with the native antibody. The development of photo-reactive crosslinker versions of protein L binding domains allows for the predictable and reproducible covalent anchoring of the blocking construct to a known location on the kappa light chain of a native antibody. This location is close enough to the antigen binding site of the native antibody to allow for effective noncovalent interaction with the tethered blocking moiety and far enough away to prevent interference to antibody binding from the protein L molecule itself. Covalently binding the protein L to the antibody ensures that the blocking moiety will be kept in close proximity to the antigen binding site. This allows for the use of blocking moieties that have a lower binding affinity for an antibody (relative to the antigen targeted by the antibody) to competitively bind with the intended target antigen at the antigen binding site of the antibody, effectively putting the antibody into an “off-state.” Blocking moiety with such relatively lower binding affinity are useful for restoring the native binding activity of an antibody quickly. A relatively lower binding affinity prevents the re-binding of cleaved blocking moiety to an antigen binding site after it dissociates from the blocking construct due to Brownian motion.

As disclosed herein, two different activation mechanisms were demonstrated for successfully cleaving a flexible tether and activating antibody. The first was a protease based cleavage technique using chymotrypsin. The inactivated cetuximab showed a ˜9 fold lower affinity than cetuximab alone, but affinity was rescued with protease treatment that cleaved the tether and activated the antibody. The second was the use of a photocleavable moiety allowing 365 nm light to cleave the linker showing a 4.2 fold reduction in binding affinity. The binding affinity for EGFR was largely restored upon light exposure. This technique was demonstrated using both the anti-FLAG antibody and the therapeutic antibody cetuximab making the technique useful for therapeutic applications to reduce side effects from systemic administration of an active antibody.

### CLOSING PARAGRAPHS

As will be understood by one of ordinary skill in the art, each embodiment disclosed herein can comprise, consist essentially of or consist of its particular stated element, step, ingredient or component. Thus, the terms “include” or “including” or “such as” are intended to be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment.

Unless otherwise indicated, all numbers expressing quantities of ingredients, properties such as molecular weight, reaction conditions, and so forth used in the specification and claims are to be understood as being modified in all instances by the term “about” or “approximately”. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value. Alternatively, “about” may mean within 1 or more than 1 standard deviation, per the practice in the given value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles, other written text, and web site content throughout this specification (referenced materials herein). Each of the referenced materials are individually incorporated herein by reference in their entirety for their referenced teaching(s), as of the filing date of the first application in the priority chain in which the specific reference was included. For instance, with regard to chemical compounds, nucleic acid, and amino acids sequences referenced herein that are available in a public database, the information in the database entry is incorporated herein by reference as of the date of an application in the priority chain in which the database identifier for that compound or sequence was first included in the text.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the example(s) or when application of the meaning renders any construction meaningless or essentially meaningless. In cases where the construction of the term would render it meaningless or essentially meaningless, the definition should be taken from Webster's Dictionary, 11th Edition or a dictionary known to those of ordinary skill in the art, such as the Oxford Dictionary of Biochemistry and Molecular Biology, 2^{nd} Edition (Ed. Anthony Smith, Oxford University Press, Oxford, 2006), and/or A Dictionary of Chemistry, 8^{th} Edition (Ed. J. Law & R. Rennie, Oxford University Press, 2020).

We claim:

1. A blocking construct for modulating binding activity of an antigen binding domain, the blocking construct comprising:
a kappa light chain-binding polypeptide comprising a Protein L amino acid sequence in which at least one amino acid residue in the Protein L amino acid sequence is substituted by a photo-reactive crosslinker residue having an activation wavelength;
a blocking moiety comprising an epitope configured to bind competitively to an antigen binding site of the antigen binding domain; and
a flexible tether, operatively connecting the kappa light chain-binding polypeptide to the blocking moiety.

2. The blocking construct of claim 1, wherein the antigen binding domain is specific for the FLAG-tag, and the epitope of the blocking moiety comprises the amino acid sequence Asp-Tyr-Lys-Asp-Asp-Asp-Asp-Lys (SEQ ID NO: 31).

3. A kappa light chain-binding polypeptide, comprising:
a set of one or more crosslinker kappa light chain-binding domains, in which a crosslinker kappa light chain-binding domain in the set comprises a Protein L amino acid sequence in which at least one amino acid residue in the Protein L amino acid sequence is substituted by a photo-reactive crosslinker residue having an activation wavelength.

4. The polypeptide of claim 3, in which the Protein L amino acid sequence is selected from the Protein L amino acid sequence set forth in any one of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10, in which the amino acid residue being substituted corresponds to position 33 of the selected amino acid sequence.

5. The polypeptide of claim 3, in which a crosslinker kappa light chain-binding domain in the set comprises the Protein L amino acid sequence set forth in in any one of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11.

6. The polypeptide of claim 3, in which the photo-reactive crosslinker residue is selected from a 4-benzoyl-L-phenylalanine (BpA) residue, a (2R)-2-amino-3-fluoro-3-(4-((2-nitrobenzyl)oxy) phenyl) propanoic acid residue (FnbY), a p-benzoyl-L-phenylalanine (pBpA), a n-(Fluoroacetyl)phenylalanine residue, a p-2′-fluoroacetyl-phenylalanine (Ffact) residue, a p-azidophenylalanine (pAzF), a p-vinylsulfonamido-(S)-phenylalanine residue, and a p-isothiocyanate phenylalanine (pNCSF) residue.

7. The polypeptide of claim 3, in which the activation wavelength of the photo-reactive crosslinker residue is 365 nm.

8. The polypeptide of claim 3, in which a crosslinker kappa light chain-binding domain in the set comprises a Protein L amino acid sequence selected from the Protein L amino acid sequence set forth in any one of: SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, or SEQ ID NO: 22.

9. The polypeptide of claim 3, in which a crosslinker kappa light chain-binding domain in the set comprises a Protein L polypeptide structure represented from N-terminus to C-terminus by the formula: β₁-L₁-β₂-α-L₂-β₃-L₃-β₄, in which:
β₁ is a first beta-sheet motif comprising a Protein L amino acid sequence selected from the Protein L amino acid sequence corresponding to positions 1 to 9 as set forth in: SEQ ID NO: 1; or SEQ ID NO: 1, in which the amino acid residue corresponding to position 6 as set forth in SEQ ID NO: 1 is substituted by alanine; or SEQ ID NO: 1, in which the amino acid residues corresponding to positions 1, 6, 8, and 9 as set forth in SEQ ID NO: 1 are substituted by, respectively, glutamine, glutamate, isoleucine, and tyrosine;
β₂ is a second beta-sheet motif comprising a Protein L amino acid sequence selected from the Protein L amino acid sequence corresponding to positions 15 to 23 as set forth in: SEQ ID NO: 1; or SEQ ID NO: 1, in which the amino acid residue corresponding to position 15 as set forth in SEQ ID NO: 1 is substituted by threonine; or SEQ ID NO: 1, in which the amino acid residues corresponding to positions 15 and 17 as set forth in SEQ ID NO: 1 are substituted by, respectively, threonine and asparagine; or SEQ ID NO: 1, in which the amino acid residues corresponding to positions 15 and 19 as set forth in SEQ ID NO: 1 are substituted by, respectively, valine and threonine;
β₃ is a third beta-sheet motif comprising a Protein L amino acid sequence selected from the Protein L amino acid sequence corresponding to positions 45 to 50 as set forth in: SEQ ID NO: 1; or SEQ ID NO: 1, in which the amino acid residues corresponding to positions 47, 49, and 50 as set forth in SEQ ID NO: 1 are substituted by, respectively, valine, valine, and alanine;
β₄ is a fourth beta-sheet motif comprising a Protein L amino acid sequence selected from the Protein L amino acid sequence corresponding to positions 55 to 61 as set forth in: SEQ ID NO: 1; or SEQ ID NO: 1, in which the amino acid residues corresponding to positions 55 and 56 as set forth in SEQ ID NO: 1 are substituted by, respectively, threonine and leucine; or SEQ ID NO: 1, in which the amino acid residues corresponding to positions 55 and 56 as set forth in SEQ ID NO: 1 are substituted by, respectively, threonine and isoleucine; or SEQ ID NO: 1, in which the amino acid residues corresponding to positions 55, 56, and 59 as set forth in SEQ ID NO: 1 are substituted by, respectively, threonine, isoleucine, and arginine;
α is an alpha helix motif comprising a Protein L amino acid sequence selected from the Protein L amino acid sequence corresponding to positions 24 to 39 as set forth in: SEQ ID NO: 1; or SEQ ID NO: 1, in which the amino acid residue corresponding to position 25 as set forth in SEQ ID NO: 1 is substituted by alanine; or SEQ ID NO: 1, in which the amino acid residues corresponding to positions 25 and 39 as set forth in SEQ ID NO: 1 are substituted by, respectively, alanine and serine; or SEQ ID NO: 1, in which the amino acid residues corresponding to positions 25, 26, 28, 29, 30, 37, and 39 as set forth in SEQ ID NO: 1 are substituted by, respectively, alanine, lysine, valine, serine, aspartate, alanine, and lysine; or SEQ ID NO: 1, in which the amino acid residues corresponding to positions 26, 29, 37, and 39 as set forth in SEQ ID NO: 1 are substituted by, respectively, lysine, serine, threonine, and lysine; or SEQ ID NO: 1, in which the amino acid residue corresponding to position 30 as set forth in SEQ ID NO: 1 is substituted by lysine; or SEQ ID NO: 1, in which the amino acid residues corresponding to positions 30 and 36 as set forth in SEQ ID NO: 1 are substituted by, respectively, lysine and asparagine; or SEQ ID NO: 1, in which the amino acid residue corresponding to position 33 as set forth in the selected amino acid sequence is substituted by a photo-reactive crosslinker residue having an activation wavelength, the photo-reactive crosslinker residue; or SEQ ID NO: 1, in which the amino acid residues corresponding to positions 37 and 39 as set forth in SEQ ID NO: 1 are substituted by, respectively, alanine and lysine;
L₁ is an amino acid linker comprising a Protein L amino acid sequence selected from the Protein L amino acid sequence corresponding to positions 10 to 14 as set forth in: SEQ ID NO: 1; or SEQ ID NO: 1, in which the amino acid residue corresponding to position 10 as set forth in SEQ ID NO: 1 is substituted by tyrosine; or SEQ ID NO: 1, in which the amino acid residue corresponding to position 11 as set forth in SEQ ID NO: 1 is substituted by glutamate; or SEQ ID NO: 1, in which the amino acid residue corresponding to position 12 as set forth in SEQ ID NO: 1 is substituted by asparagine; or SEQ ID NO: 1, in which the amino acid residue corresponding to position 13 as set forth in SEQ ID NO: 1 is substituted by serine;
L₂ is an amino acid linker comprising a Protein L amino acid sequence selected from the Protein L amino acid sequence corresponding to positions 40 to 44 as set forth in: SEQ ID NO: 1; or SEQ ID NO: 1, in which the amino acid residue corresponding to position 41 as set forth in SEQ ID NO: 1 is substituted by aspartate; or SEQ ID NO: 1, in which the amino acid residue corresponding to position 41 as set forth in SEQ ID NO: 1 is substituted by glutamate; or SEQ ID NO: 1, in which the amino acid residues corresponding to positions 41 and 44 as set forth in SEQ ID NO: 1 are substituted by, respectively, glutamate and lysine; or SEQ ID NO: 1, in which the amino acid residues corresponding to positions 41, 42, and 44 as set forth in SEQ ID NO: 1 are substituted by, respectively, glutamate, histidine, and lysine; and
L₃ is an amino acid linker comprising a Protein L amino acid sequence selected from the Protein L amino acid sequence corresponding to positions 51 to 54 as set forth in: SEQ ID NO: 1; or SEQ ID NO: 1, in which the amino acid residue corresponding to position 52 as set forth in SEQ ID NO: 1 is substituted by lysine.

10. The polypeptide of claim 3, in which β1 is a first beta-sheet motif comprising a Protein L amino acid sequence selected from the Protein L amino acid sequence corresponding to positions 1 to 9 as set forth in SEQ ID NO 1.

11. The polypeptide of claim 10, in which:
the amino acid residue corresponding to position 6 as set forth in SEQ ID NO: 1 is substituted by alanine; or
the amino acid residues corresponding to positions 1, 6, 8, and 9 as set forth in SEQ ID NO: 1 are substituted by, respectively, glutamine, glutamate, isoleucine, and tyrosine.

12. The polypeptide of claim 3, in which β2 is a second beta-sheet motif comprising a Protein L amino acid sequence selected from the Protein L amino acid sequence corresponding to positions 15 to 23 as set forth in SEQ ID NO: 1.

13. The polypeptide of claim 12, in which:
the amino acid residue corresponding to position 15 as set forth in SEQ ID NO: 1 is substituted by threonine or
the amino acid residues corresponding to positions 15 and 17 as set forth in SEQ ID NO: 1 are substituted by, respectively, threonine and asparagine; or
the amino acid residues corresponding to positions 15 and 19 as set forth in SEQ ID NO: 1 are substituted by, respectively, valine and threonine.

14. The polypeptide of claim 3, in which 33 is a third beta-sheet motif comprising a Protein L amino acid sequence selected from the Protein L amino acid sequence corresponding to positions 45 to 50 as set forth in SEQ ID NO: 1

15. The polypeptide of claim 14, in which the amino acid residues corresponding to positions 47, 49, and 50 as set forth in SEQ ID NO: 1 are substituted by, respectively, valine, valine, and alanine.

16. The polypeptide of claim 3, in which 34 is a fourth beta-sheet motif comprising a Protein L amino acid sequence selected from the Protein L amino acid sequence corresponding to positions 55 to 61 as set forth in SEQ ID NO: 1.

17. The polypeptide of claim 16, in which:
the amino acid residues corresponding to positions 55 and 56 as set forth in SEQ ID NO: 1 are substituted by, respectively, threonine and leucine; or
the amino acid residues corresponding to positions 55 and 56 as set forth in SEQ ID NO: 1 are substituted by, respectively, threonine and isoleucine; or
the amino acid residues corresponding to positions 55, 56, and 59 as set forth in SEQ ID NO: 1 are substituted by, respectively, threonine, isoleucine, and arginine.

18. The polypeptide of claim 3, in which α is an alpha helix motif comprising a Protein L amino acid sequence selected from the Protein L amino acid sequence corresponding to positions 24 to 39 as set forth in SEQ ID NO: 1.

19. The polypeptide of claim 18, in which:
the amino acid residue corresponding to position 25 as set forth in SEQ ID NO: 1 is substituted by alanine; or
the amino acid residues corresponding to positions 25 and 39 as set forth in SEQ ID NO: 1 are substituted by, respectively, alanine and serine; or
the amino acid residues corresponding to positions 25, 26, 28, 29, 30, 37, and 39 as set forth in SEQ ID NO: 1 are substituted by, respectively, alanine, lysine, valine, serine, aspartate, alanine, and lysine; or
the amino acid residues corresponding to positions 26, 29, 37, and 39 as set forth in SEQ ID NO: 1 are substituted by, respectively, lysine, serine, threonine, and lysine; or
the amino acid residue corresponding to position 30 as set forth in SEQ ID NO: 1 is substituted by lysine; or
the amino acid residues corresponding to positions 30 and 36 as set forth in SEQ ID NO: 1 are substituted by, respectively, lysine and asparagine; or
the amino acid residues corresponding to positions 30 and 36 as set forth in SEQ ID NO: 1 are substituted by, respectively, lysine and asparagine; or
the amino acid residue corresponding to position 33 as set forth in the selected amino acid sequence is substituted by a photo-reactive crosslinker residue having an activation wavelength; or
the amino acid residues corresponding to positions 37 and 39 as set forth in SEQ ID NO: 1 are substituted by, respectively, alanine and lysine.

20. The polypeptide of claim 3, in which L₁ is an amino acid linker comprising a Protein L amino acid sequence selected from the Protein L amino acid sequence corresponding to positions 10 to 14 as set forth in SEQ ID NO: 1.

21. The polypeptide of claim 20, in which:
the amino acid residue corresponding to position 10 as set forth in SEQ ID NO: 1 is substituted by tyrosine; or
the amino acid residue corresponding to position 11 as set forth in SEQ ID NO: 1 is substituted by glutamate; or
the amino acid residue corresponding to position 12 as set forth in SEQ ID NO: 1 is substituted by asparagine; or
the amino acid residue corresponding to position 13 as set forth in SEQ ID NO: 1 is substituted by serine.

22. The polypeptide of claim 3, in which L₂ is an amino acid linker comprising a Protein L amino acid sequence selected from the Protein L amino acid sequence corresponding to positions 40 to 44 as set forth in SEQ ID NO: 1.

23. The polypeptide of claim 22, in which:
the amino acid residue corresponding to position 41 as set forth in SEQ ID NO: 1 is substituted by aspartate; or
the amino acid residue corresponding to position 41 as set forth in SEQ ID NO: 1 is substituted by glutamate; or
the amino acid residues corresponding to positions 41 and 44 as set forth in SEQ ID NO: 1 are substituted by, respectively, glutamate and lysine; or
the amino acid residues corresponding to positions 41 and 44 as set forth in SEQ ID NO: 1 are substituted by, respectively, glutamate and lysine.

24. The polypeptide of claim 3, in which L₃ is an amino acid linker comprising a Protein L amino acid sequence selected from the Protein L amino acid sequence corresponding to positions 51 to 54 as set forth in SEQ ID NO: 1.

25. The polypeptide of claim 3, in which L₃ is an amino acid linker comprising a Protein L amino acid sequence selected from the Protein L amino acid sequence corresponding to positions 51 to 54 as set forth in SEQ ID NO: 1, in which the amino acid residue corresponding to position 52 as set forth in SEQ ID NO: 1 is substituted by lysine.

26. The polypeptide of claim 3, in which a crosslinker kappa light chain-binding domain in the set comprises an engineered Protein L kappa light chain-binding domain comprising a crosslinker alpha helix motif having a structure represented from N-terminus to C-terminus by the amino acid sequence set forth in any of: Phe-Glu-Glu-Ala-Thr-Ala-Glu-Ala-Tyr-X-Tyr-Ala-Asp-Leu-Leu-Ala (SEQ ID NO: 23); Phe-Ala-Lys-Ala-Val-Ser-Asp-Ala-Tyr-X-Tyr-Ala-Asp-Ala-Leu-Lys (SEQ ID NO: 24); Phe-Glu-Glu-Ala-Thr-Ala-Lys-Ala-Tyr-X-Tyr-Ala-Asp-Leu-Leu-Ala (SEQ ID NO: 25); Phe-Glu-Glu-Ala-Thr-Ala-Lys-Ala-Tyr-X-Tyr-Ala-Asn-Leu-Leu-Ala (SEQ ID NO: 26); Phe-Glu-Lys-Ala-Thr-Ser-Glu-Ala-Tyr-X-Tyr-Ala-Asp-Thr-Leu-Lys (SEQ ID NO: 27); Phe-Glu-Glu-Ala-Thr-Ala-Glu-Ala-Tyr-X-Tyr-Ala-Asp-Ala-Leu-Lys (SEQ ID NO: 28); Phe-Ala-Glu-Ala-Thr-Ala-Glu-Ala-Tyr-X-Tyr-Ala-Asp-Leu-Leu-Ala (SEQ ID NO: 29); and Phe-Ala-Glu-Ala-Thr-Ala-Glu-Ala-Tyr-X-Tyr-Ala-Asp-Leu-Leu-Ser (SEQ ID NO: 30), and in which the amino acid residue corresponding to position X is substituted by the photo-reactive crosslinker residue.

27. The polypeptide of claim 3, in which a crosslinker kappa light chain-binding domain in the set comprises an engineered Protein L kappa light chain-binding domain selected from a domain C*, a domain C₁, a domain C₂, a domain C₃, a domain C₄, a domain B1, a domain B2, a domain B3, a domain B4, and a domain B5, in which the selected Protein L kappa light chain-binding domain comprises the photo-reactive crosslinker residue.

28. The polypeptide of claim 3, in which the engineered Protein L kappa light chain-binding domain is:
a domain C* comprising a photo-reactive crosslinker residue;
a domain C₁ comprising a photo-reactive crosslinker residue;
a domain C₂ comprising a photo-reactive crosslinker residue;
a domain C₃ comprising a photo-reactive crosslinker residue;
a domain C₄ comprising a photo-reactive crosslinker residue;
a domain B1 comprising a photo-reactive crosslinker residue;
a domain B2 comprising a photo-reactive crosslinker residue;
a domain B3 comprising a photo-reactive crosslinker residue;
a domain B4 comprising a photo-reactive crosslinker residue; or
a domain B5 comprising a photo-reactive crosslinker residue.

29. The polypeptide of claim 3, in which the photo-reactive crosslinker residue is selected from a 4-benzoyl-L-phenylalanine (BpA) residue, a (2R)-2-amino-3-fluoro-3-(4-((2-nitrobenzyl)oxy) phenyl) propanoic acid residue (FnbY), a p-benzoyl-L-phenylalanine (pBpA), a n-(Fluoroacetyl)phenylalanine residue, a p-2′-fluoroacetyl-phenylalanine (Ffact) residue, a p-azidophenylalanine (pAzF), a p-vinylsulfonamido-(S)-phenylalanine residue, and a p-isothiocyanate phenylalanine (pNCSF) residue.

30. The polypeptide of claim 29, in which the activation wavelength of the photo-reactive crosslinker residue is 365 nm.

31. A blocking construct for modulating the binding activity of an antigen binding domain, the blocking construct comprising:
the kappa light chain-binding polypeptide of any of claims 3-30; which is
operatively connected via a flexible tether to a blocking moiety that is configured to bind to antigen binding site of the antigen binding domain.

32. A blocking construct for modulating the binding activity of an antigen binding domain, the blocking construct comprising:
a kappa light chain-binding polypeptide comprising:
a set of one or more crosslinker kappa light chain-binding domains, in which a crosslinker kappa light chain-binding domain in the set comprises a Protein L amino acid sequence in which at least one amino acid residue in the Protein L amino acid sequence is substituted by a photo-reactive crosslinker residue having an activation wavelength; which is
operatively connected via a flexible tether to a blocking moiety that is configured to bind to antigen binding site of the antigen binding domain.

33. The blocking construct of claim 1 or claim 32, in which:
the kappa light chain-binding polypeptide is configured to have, when in the proximity of a kappa light chain of an antigen binding domain, a binding interaction with the kappa light chain and thereby form a non-covalent bond between the blocking construct and the antigen binding domain, and, upon exposure of a photo-reactive crosslinker residue of a kappa light chain-binding domain forming the non-covalent bond to light of its activation wavelength, to activate the photo-reactive crosslinker residue and crosslink the kappa light chain-binding domain forming the non-covalent bond to the kappa light chain and thereby form a covalent bond between the blocking construct and antigen binding domain;
the blocking moiety comprises an epitope configured to competitively bind to an antigen binding site of the antigen binding domain; and
the flexible tether comprises a flexible linker operatively connected at a proximal end to the kappa light chain-binding polypeptide and at a distal end to the blocking moiety, the flexible linker configured to have an end-to-end length to tether the blocking moiety at a sufficient movement radius for the blocking moiety to establish an intramolecular binding interaction between its epitope and the antigen binding site and to establish an effective concentration of the blocking moiety at the antigen binding site, thereby to facilitate the competitive binding of the blocking moiety at the antigen binding site and modulate the binding activity of the antigen binding domain.

34. The blocking construct of claim 1 or claim 32, in which the blocking moiety comprises a polypeptide, a oligonucleotide, a glycoprotein, a fusion protein, an engineered protein, or any fragment or combination thereof.

34. The blocking construct of claim 1 or claim 32, in which the blocking moiety further comprises a cleavable linker configured to cleave upon its activation by a trigger.

35. The blocking construct of claim 34, in which the blocking moiety is:
a polypeptide blocking moiety, and the cleavable linker is a protease cleavage site configured to cleave upon its activation by a protease enzyme trigger, whereby upon activation of the protease cleavage site by the protease enzyme trigger, the protease cleavage site cleaves the blocking construct at the protease cleavage site; or
a polypeptide, and the cleavable linker is a photo-cleavable linker having an activation wavelength, the photo-cleavable linker configured to cleave upon its activation by exposure to light of the activation wavelength, whereby upon activation of the photo-cleavable linker, the photo-cleavable linker cleaves the blocking construct at the position of photo-cleavable linker.

36. The blocking construct of claim 35, in which the photo-cleavable linker (if present) is a Fmoc cleavable linker.

37. The blocking construct of claim 36, in which the Fmoc cleavable linker is positioned:
at the N-terminus of the polypeptide; or
at the C-terminus of the polypeptide.

38. The blocking construct of claim 1 or claim 32, in which the blocking construct is crosslinked to an antigen binding domain, whereby, upon activation of the cleavable linker by the trigger, the cleavable linker cleaves the blocking construct at the cleavable linker to dissociate the epitope of the blocking moiety from the blocking construct and thereby decrease the effective concentration of the blocking moiety at the antigen binding site to further modulate the binding activity of the antigen binding domain.

39. The blocking construct of claim 1 or claim 32, in which the epitope of the blocking moiety is selected from any of the group consisting of:
a FLAG epitope comprising the amino acid sequence Asp-Tyr-Lys-Asp-Asp-Asp-Asp-Lys (SEQ ID NO: 31); and
a EGFR epitope comprising the amino acid sequence Gln-Gly-Gln-Ser-Gly-Gln-Cys-Ile-Ser-Pro-Arg-Gly-Cys-Pro-Asp-Gly-Pro-Tyr-Val-Met-Tyr (SEQ ID NO: 32).

40. The blocking construct of claim 1 or claim 32, in which the epitope of the blocking moiety is:
a FLAG epitope comprising the amino acid sequence Asp-Tyr-Lys-Asp-Asp-Asp-Asp-Lys (SEQ ID NO: 31); or
an EGFR epitope comprising the amino acid sequence Gln-Gly-Gln-Ser-Gly-Gln-Cys-Ile-Ser-Pro-Arg-Gly-Cys-Pro-Asp-Gly-Pro-Tyr-Val-Met-Tyr (SEQ ID NO: 32).

41. The blocking construct of claim 1 or claim 32, in which the kappa light chain-binding polypeptide, the blocking moiety, or the flexible tether further comprise a conjugation moiety.

42. The blocking construct of claim 41, wherein the conjugation moiety comprises a sortase recognition site comprising the amino acid sequence Leu-Pro-Glu-Thr-Gly (SEQ ID NO: 33), or a click chemistry residue.

43. The blocking construct of claim 1 or claim 32, in which the flexible tether comprises at least one of:
a polypeptide flexible linker having a structure represented from N-terminus to C-terminus by the formula: (G₄S)-(EA₃K)₄-(G₄S)-(EA₃K)₄-(G₄S)-(X) (SEQ ID NO: 34), in which X is a Sortase A recognition site comprising the amino acid sequence: Leu-Pro-Glu-Thr-Gly (SEQ ID NO: 33);
a polypeptide flexible linker having a structure represented from N-terminus to C-terminus by the formula: (G₄S)-(EA₃K)₄-(G₄S)-(EAAAK)₄-(G₄S) (SEQ ID NO: 35);
a polypeptide flexible linker having a structure represented from N-terminus to C-terminus by the formula: (G₂S)-(EA₃K)₄-(G₂S)-(EA₃K)₄-(G₂S)-(X) (SEQ ID NO: 36), in which X is a Sortase A recognition site comprising the amino acid sequence: Leu-Pro-Glu-Thr-Gly (SEQ ID NO: 33); and
a polypeptide flexible linker having a structure represented from N-terminus to C-terminus by the formula: (G₂S)-(EA₃K)₂-(G₂S)-(EA₃K)₄-(G₂S) (SEQ ID NO: 37).

44. The blocking construct of claim 1 or claim 32, in which the flexible linker comprises at least one of:
a polypeptide flexible linker having a structure represented from N-terminus to C-terminus by the formula: (G₄S)-(EA₃K)₄-(G₄S)-(EA₃K)₄-(G₄S)-(X) (SEQ ID NO: 34), in which X is a Sortase A recognition site comprising the amino acid sequence: Leu-Pro-Glu-Thr-Gly (SEQ ID NO: 33);
a polypeptide flexible linker having a structure represented from N-terminus to C-terminus by the formula: (G₄S)-(EA₃K)₄-(G₄S)-(EAAAK)₄-(G₄S) (SEQ ID NO: 35);
a polypeptide flexible linker having a structure represented from N-terminus to C-terminus by the formula: (G₂S)-(EA₃K)₄-(G₂S)-(EA₃K)₄-(G₂S)-(X) (SEQ ID NO: 36) in which X is a Sortase A recognition site comprising the amino acid sequence Leu-Pro-Glu-Thr-Gly (SEQ ID NO: 33); or
a polypeptide flexible linker having a structure represented from N-terminus to C-terminus by the formula: (G₂S)-(EA₃K)₄-(G₂S)-(EA₃K)₄-(GYS) (SEQ ID NO: 37).

45. The blocking construct of claim 1 or claim 32, in which the flexible tether further comprises a cleavable linker configured to cleave upon its activation by a trigger.

46. The blocking construct of claim 45, in which the cleavable linker comprises:
a protease cleavage site configured to cleave upon its activation by a protease enzyme trigger, whereby upon activation of the protease cleavage site by the protease enzyme trigger, the protease cleavage site cleaves the blocking construct at the protease cleavage site; or
a photo-cleavable linker having an activation wavelength, the photo-cleavable linker configured to cleave upon its activation by exposure to light of the activation wavelength, whereby upon activation of the photo-cleavable linker, the photo-cleavable linker cleaves the blocking construct at the position of the photo-cleavable linker.

47. The blocking construct of claim 46, in which the photo-cleavable linker (if present) is a Fmoc cleavable linker.

48. The blocking construct of claim 47, in which the Fmoc cleavable linker is positioned at:
the N-terminus of the polypeptide; or
the C-terminus of the polypeptide.

49. The blocking construct of claim 1 or claim 32, in which the activation wavelength of the photo-cleavable linker is 365 nm.

50. The blocking construct of claim 1 or claim 32, in which the flexible tether comprises a flexible portion and a rigid portion.

51. The blocking construct of claim 50, in which the flexible tether comprises one or more repeating motifs of the structure (X—Y)ₙ, in which X and Y are, respectively a flexible portion operatively connected to a rigid portion, and n is the number repeats.

52. The blocking construct of claim 51, in which n equals one, two, three, four, five, six, seven, eight, nine, ten, eleven, twelve, thirteen, fourteen, fifteen, sixteen, seventeen, eighteen, nineteen, or twenty.

53. The blocking construct of claim 51, in which the flexible portion is one of:
a (G₂S) flexible portion comprising the amino acid sequence Gly-Gly-Ser;
a (G₃S) flexible portion comprising the amino acid sequence Gly-Gly-Gly-Ser; or
a (G₄S) flexible portion comprising the amino acid sequence Gly-Gly-Gly-Gly-Ser (SEQ ID NO: 84).

54. The construct of claim 501, in which the rigid portion is a (EA₃K)₄ rigid portion comprising the amino acid sequence Glu-Ala-Ala-Ala-Lys-Glu-Ala-Ala-Ala-Lys-Glu-Ala-Ala-Ala-Lys-Glu-Ala-Ala-Ala-Lys (SEQ ID NO: 85).

55. The blocking construct of claim 1 or claim 32, in which the end-to-end length of the flexible tether is configured to be of from 1.0 angstrom (Å) to 5.0 Å, of from 5.0 Å to 10.0 Å, of from 10.0 Å to 15.0 Å, of from 15.0 Å to 20.0 Å, of from 20.0 Å to 25.0 Å, of from 25.0 Å to 30.0 Å, of from 30.0 Å to 35.0 Å, of from 35.0 Å to 40.0 Å, of from 45.0 Å to 50.0 Å, of from 50.0 Å to 55.0 Å, of from 55.0 Å to 60.0 Å, of from 60.0 Å to 65.0 Å, of from 65.0 Å to 70.0 Å, of from 75.0 Å to 80.0 Å, of from 80.0 Å to 85.0 Å, of from 85.0 Å to 90.0 Å, of from 95.0 Å to 100.0 Å, of from 105.0 Å to 110.0 Å, of from 115.0 Å to 120.0 Å, of from 125.0 Å to 130.0 Å, of from 135.0 Å to 140.0 Å, of from 140.0 Å to 145.0 Å, and of from 145.0 Å to 150.0 Å.

56. The blocking construct of claim 1 or claim 32, in which the rigid portion has a persistence length of from 1.0 angstrom (Å) to 2.0 Å, of from 2.0 Å to 3.0 Å, of from 2.0 Å to 3.0 Å, of from 3.0 Å to 4.0 Å, of from 4.0 Å to 5.0 Å, of from 6.0 Å to 7.0 Å, of from 7.0 Å to 8.0 Å, of from 8.0 Å to 9.0 Å, of from 9.0 Å to 10.0 Å, of from 10.0 Å to 11.0 Å, of from 12.0 Å to 13.0 Å, of from 13.0 Å to 14.0 Å, of from 14.0 Å to 15.0 Å, of from 16.0 Å to 17.0 Å, of from 17.0 Å to 18.0 Å, of from 18.0 Å to 19.0 Å, and of from 19.0 Å to 20.0 Å.

57. The kappa light chain-binding polypeptide of claim 3, in which the kappa light chain-binding polypeptide is configured to have a binding interaction with the kappa light chain of an antigen binding domain derived from, or forming any portion of, an antibody or antibody fragment selected from, an immunoglobulin, an IgA isotype antibody, an IgD isotype antibody, an IgE isotype antibody, an IgG isotype antibody, an IgM isotype antibody, a monospecific antibody, a bispecific antibody, a Fab fragment, a Fab′ fragment, an F(ab′)₂ fragment, an Fv fragment, a rigG fragment, a scFv fragment, a scFV-Fc fragment, and a minibody fragment.

58. The blocking construct of claim 1 or claim 32, in which the antigen binding domain is derived from, or forms any portion of an antibody selected from an alemtuzumab, a bevacizumab, a cetuximab, an edrecolomab, a gemtuzumab, an ibritumomab tiuxetan, a matuzumab, a panitumumab, a rituximab, and a trastuzumab.

59. A blocked immunoglobulin complex comprising: an immunoglobulin crosslinked to a set of one or more blocking constructs.

60. The blocked immunoglobulin complex of claim 59, in which the immunoglobulin is an antibody selected from an alemtuzumab, a bevacizumab, a cetuximab, an edrecolomab, a gemtuzumab, an ibritumomab tiuxetan, a matuzumab, a panitumumab, a rituximab, a trastuzumab, and an anti-FLAG antibody.

61. The blocked immunoglobulin complex of claim 60, in which a blocking construct in the set is selected from:
any of the blocking constructs of claim 31-56 or 58, in which the epitope of the blocking moiety of the blocking construct is an EGFR epitope comprising the amino acid sequence Gln-Gly-Gln-Ser-Gly-Gln-Cys-Ile-Ser-Pro-Arg-Gly-Cys-Pro-Asp-Gly-Pro-Tyr-Val-Met-Tyr (SEQ ID NO: 32); or
any of the blocking constructs of claim 1, 31-56, or 58, in which the epitope of the blocking moiety of the blocking construct is a FLAG epitope comprising the amino acid sequence Asp-Tyr-Lys-Asp-Asp-Asp-Asp-Lys (SEQ ID NO: 31).

62. The blocked immunoglobulin complex of claim 59, in which the immunoglobulin comprises an antigen binding domain derived from, or forming any portion of, an antibody or antibody fragment.

63. The blocked immunoglobulin complex of claim 62, in which the antigen binding domain is derived from, or forms any portion of an immunoglobulin, an IgA isotype antibody, an IgD isotype antibody, an IgE isotype antibody, an IgG isotype antibody, an IgM isotype antibody, a monospecific antibody, a bispecific antibody, a Fab fragment, a Fab′ fragment, an F(ab′)₂ fragment, an Fv fragment, a rigG fragment, a scFv fragment, a scFV-Fc fragment, and a minibody fragment.

64. A blocked immunoglobulin complex, comprising:
a heavy chain comprising SEQ ID NO: 42; and
a light chain comprising SEQ ID NO: 43 which is crosslinked to the blocking construct of any of claim 31-56 or 58.

65. A pharmaceutical composition comprising the blocked immunoglobulin complex of any one of claims 59-64.

66. The pharmaceutical composition of claim 65, further comprising a pharmaceutical excipient.

67. A method of treating cancer, comprising: administering a therapeutically effective amount of the blocked immunoglobulin complex of claim 59 to a subject in need thereof.

68. The method of claim 67, in which the immunoglobulin of the blocked immunoglobulin complex is cetuximab and thereby forms a cetuximab blocked immunoglobulin complex.

69. The method of claim 67, in which the method is a treatment of head and neck cancer in a subject including a first line treatment, a second line treatment, a locoregional head and neck cancer, and a metastatic head and neck cancer.

70. The method of claim 67, in which the method comprises at least one of:
the treatment of head and neck cancers associated with high expression of EGFR, including hypopharyngeal cancer, laryngeal cancer, lip and oral cavity cancer, metastatic squamous neck cancer, nasopharyngeal cancer, oropharyngeal cancer, paranasal sinus and nasal cavity cancer, and salivary gland cancer; or
treatment of squamous cell carcinoma of the head and neck in a subject, the method comprising administering to the subject in need thereof a pharmaceutically or therapeutically useful amount of the blocked immunoglobin complex; or
treatment of squamous cell carcinoma of the head and neck in a subject, the method comprising administering to the subject in need thereof: a pharmaceutically or therapeutically useful amount of the cetuximab blocked immunoglobulin complex; and a pharmaceutically or therapeutically useful amount of monalizumab; or
treatment of squamous cell carcinoma of the head and neck in a subject, the method comprising administering to the subject in need thereof: a pharmaceutically or therapeutically useful amount of the cetuximab blocked immunoglobulin complex; and a pharmaceutically or therapeutically useful amount of ficlatuzumab; or
treatment of squamous cell carcinoma of the head and neck in a subject, the method comprising administering to the subject in need thereof: a pharmaceutically or therapeutically useful amount of the cetuximab blocked immunoglobulin complex; and a pharmaceutically or therapeutically useful amount of palbociclib, or a pharmaceutically acceptable salt thereof; or
treatment of squamous cell carcinoma of the head and neck in a subject, the method comprising administering to the subject in need thereof: a pharmaceutically or therapeutically useful amount of the cetuximab blocked immunoglobulin complex; and a pharmaceutically or therapeutically useful amount of cabozantinib, or a pharmaceutically acceptable salt thereof; or
squamous cell carcinoma of the head and neck in a subject, the method comprising administering to the subject in need thereof: a pharmaceutically or therapeutically useful amount of the cetuximab blocked immunoglobulin complex; and a pharmaceutically or therapeutically useful amount of penpulimab; or
treatment of squamous cell carcinoma of the head and neck in a subject, the method comprising administering to the subject in need thereof: a pharmaceutically or therapeutically useful amount of the cetuximab blocked immunoglobulin complex; and a pharmaceutically or therapeutically useful amount of pembrolizumab; or
treatment of squamous cell carcinoma of the head and neck in a subject, the method comprising administering to the subject in need thereof: a pharmaceutically or therapeutically useful amount of the cetuximab blocked immunoglobulin complex; and a pharmaceutically or therapeutically useful amount of 5-Fluorouracil (5-FU); and a pharmaceutically or therapeutically useful amount of an agent selected from the group of cisplatin and carboplatin, or a pharmaceutically acceptable salt thereof; or
treatment of squamous cell carcinoma of the head and neck in a subject, the method comprising administering to the subject in need thereof: a pharmaceutically or therapeutically useful amount of the cetuximab blocked immunoglobulin complex; and a pharmaceutically or therapeutically useful amount of paclitaxel, or a pharmaceutically acceptable salt thereof; and
a pharmaceutically or therapeutically useful amount of carboplatin, or a pharmaceutically acceptable salt thereof.

71. The method of claim 67, in which the method includes treatment of colon cancer, including metastatic colorectal cancer, in which the cancer cells express epidermal growth factor receptor (EGFR) protein, in a subject, the method comprising administering to the subject in need thereof a pharmaceutically or therapeutically useful amount of the blocked immunoglobulin complex.

72. The method of claim 71, in which the method of treatment includes:
RAS wild-type (WT) metastatic colorectal cancer in a subject in need thereof, the method comprising administering to the subject a pharmaceutically or therapeutically useful amount of the blocked immunoglobulin complex; or
RAS wild-type (WT) metastatic colorectal cancer in a subject in need thereof, the method comprising administering to the subject a pharmaceutically or therapeutically useful amount of each of: the blocked immunoglobulin complex; and one or more chemotherapeutic agents selected from the group of oxaliplatin, irinotecan, regorafenib, trifluridin tipiracil (TAS-102), pembrolizumab, afatinib, tepotinib, leucovorin, 5-fluorouracil, capecitabine, bevacizumab, ziv-aflibercept, ramucirumab, panitumumab, leucovorin, and Trifluridine with tipiracil; or
treatment of metastatic colorectal cancer in a subject in need thereof, the method comprising administering to the subject a pharmaceutically or therapeutically useful amount of each of: the blocked immunoglobulin complex; and one or more anticancer agents selected from the group of leucovorin, 5-FU, and oxaliplatin, or a pharmaceutically acceptable salt thereof; or
treatment of metastatic colorectal cancer in a subject in need thereof, the method comprising administering to the subject a pharmaceutically or therapeutically useful amount of each of: the blocked immunoglobulin complex; and one or more anticancer agents selected from the group of leucovorin, 5-FU, and irinotecan, or a pharmaceutically acceptable salt thereof; or
treatment of metastatic colorectal cancer in a subject in need thereof, the method comprising administering to the subject a pharmaceutically or therapeutically useful amount of each of: the blocked immunoglobulin complex; and one or more anticancer agents selected from the group of capecitabine and oxaliplatin, or a pharmaceutically acceptable salt thereof; or
treatment of metastatic colorectal cancer in a subject in need thereof, the method comprising administering to the subject a pharmaceutically or therapeutically useful amount of each of: the blocked immunoglobulin complex; and one or more anticancer agents selected from the group of leucovorin, 5-FU, oxaliplatin, and irinotecan, or a pharmaceutically acceptable salt thereof; or
treatment of RAS wild-type (WT) metastatic colorectal cancer in a subject in need thereof, the method comprising administering to the subject a pharmaceutically or therapeutically useful amount of each of: the blocked immunoglobulin complex; and afatinib, or a pharmaceutically acceptable salt thereof; or
treatment of RAS wild-type (WT) metastatic colorectal cancer in a subject in need thereof, the method comprising administering to the subject a pharmaceutically or therapeutically useful amount of each of: the blocked immunoglobulin complex; and tefotinib, or a pharmaceutically acceptable salt thereof; or
treatment of colorectal cancer in a subject, the method comprising administering to the subject in need thereof a pharmaceutically or therapeutically useful amount of each of: the blocked immunoglobulin complex, as described herein; and encorafenib, or a pharmaceutically acceptable salt thereof; or
treatment of colorectal cancer in a subject in need thereof, the method comprising administering to the subject a pharmaceutically or therapeutically useful amount of each of: the blocked immunoglobulin complex; encorafenib, or a pharmaceutically acceptable salt thereof; and binimetinib, or a pharmaceutically acceptable salt thereof; or
treatment of RAS wild-type (WT) metastatic colorectal cancer in a subject in need thereof, the method comprising administering to the subject a pharmaceutically or therapeutically useful amount of each of: the blocked immunoglobulin complex; vemurafenib, or a pharmaceutically acceptable salt thereof; and camrelizumab; or
treatment of metastatic colorectal adenocarcinoma with mutant APC, mutant TP53 and mutant KRAS genes in a subject, the method comprising administering to the subject in need thereof a pharmaceutically or therapeutically useful amount of the blocked immunoglobulin complex.

73. The method claim 67, comprising use of the blocked immunoglobulin complex in a method of treatment of colon cancer, including metastatic colorectal cancer, in which the cancer cells contain at least one gene mutation selected from the group consisting of: a K-RAS (RAS) gene mutation, a RAF gene mutation, and a PI3K gene mutation in a subject, the method comprising administering to the subject in need thereof a pharmaceutically or therapeutically useful amount of the blocked immunoglobulin complexes.

74. The method of claim 73, in which one or more of:
the K-RAS mutations include G12A, G12C, G12D, G12R, G12S, G12V, G13A, G13C, G13D, G13S, G13V, A146P, A146T, A146V, Q61H, Q61L, Q61R, and K117N mutations; or
the method includes treating colon cancer with a K-RAS mutation present, including metastatic colon cancer with a K-RAS mutation present, in a subject, the method comprising administering to the subject a pharmaceutically or therapeutically effective amount of each of: the blocked immunoglobulin complex, and panitumumab; or
the blocked immunoglobulin complex is used in a method of treatment of colon cancer, including metastatic colorectal cancer, in which the cancer cells overexpress EGFR ligand, in a subject, the method comprising administering to the subject in need thereof a pharmaceutically or therapeutically useful amount of the blocked immunoglobulin complex; or
method includes treatment of metastatic colorectal cancer in a subject, the method comprising administering to the subject in need thereof a pharmaceutically or therapeutically useful amount of each of: the blocked immunoglobulin complex; irinotecan, or a pharmaceutically acceptable salt thereof; oxaliplatin, or a pharmaceutically acceptable salt thereof; and 5-fluorouracil, or a pharmaceutically acceptable salt thereof.

75. A method of modifying the binding activity of antigen binding domain comprising:
providing a set of one or more blocking constructs of any of claim 1, 31-56, or 58; and
crosslinking the set of one or more blocking constructs to an antigen binding domain to thereby modify the binding activity of the antigen binding domain.

76. The method of claim 75, further comprising exposing the set of one or more blocking constructs to an ultraviolet light trigger to activate the cleavable linker of the blocking construct to disassociate the blocking moiety from the antigen binding domain and reduce the effective concentration of block moiety at the antigen binding domain to thereby modify the binding activity antigen binding domain to an antigen.

77. The method of claim 76, in which the ultraviolet light trigger has an activation wavelength of 365 nm.

78. A method for producing a kappa light chain-binding polypeptide, comprising:
expressing a nucleic acid sequence encoding the kappa light chain-binding polypeptide amino acid sequence of the kappa light chain-binding polypeptide of any of claim 3-30 or 57 in transformant cells to produce the kappa light chain-binding polypeptide; and
extracting and purifying the produced kappa light chain-binding polypeptide from the transformant cells.

79. A method for producing a blocking construct, comprising:
expressing a nucleic acid sequence encoding the amino acid sequence of the blocking construct of any of claim 1, 31-56, or 58 in transformant cells to produce the blocking construct; and
extracting and purifying the produced blocking construct from the transformant cells.

80. The method of claim 79, in which one or more of:
the transformant cells comprise Escherichia coli (E. coli) bacteria;
the transformant cells comprise BL21 (DE3) strain E. coli bacteria;
further comprising: growing the transformant cells in lysogeny broth (LB) for 12 hours or more at 37° C.; and diluting the LB 100 fold;
the nucleic acid sequence is a codon optimized amino acid sequence optimized for expression in (E. coli) bacteria;
the amino acid sequence is codon optimized by: amplifying both a vector and an insert with PCR primers containing compatible 5′ overhangs; and assembling the vector and the insert via a NEB HiFi assembly reaction;
the nucleic acid sequence is introduced into the cells for expression by a vector; or
the nucleic acid sequence is introduced into the cells using a pET21b(+) expression vector.

81. A method for producing a blocked immunoglobulin complex, comprising:
expressing a nucleic acid sequence encoding the amino acid sequence of the immunoglobulin of the blocked immunoglobulin complex of any of claims 59-64 in transformant cells to produce the immunoglobulin;
expressing a nucleic acid sequence encoding the amino acid sequence of the blocking construct of any of claim 1, 2, 31-56, or 58 in the transformant cells to produce the blocking construct;
extracting and purifying the immunoglobulin and the blocking construct from the transformant cells; and
exposing the immunoglobulin and blocking constructs to a crosslinker trigger to crosslink the immunoglobulin to the blocking constructs and thereby produce blocked immunoglobulin complex.

82. The method of claim 81, in which one or more of:
the transformant cells comprise Escherichia coli (E. coli) bacteria;
the transformant cells comprise BL21 (DE3) strain E. coli bacteria;
further comprising: growing the transformant cells in lysogeny broth (LB) for 12 hours or more at 37° C.; and diluting the LB 100 fold;
the nucleic acid sequence is a codon optimized amino acid sequence optimized for expression in (E. coli) bacteria;
the amino acid sequence is codon optimized by: amplifying both a vector and an insert with PCR primers containing compatible 5′ overhangs; and assembling the vector and the insert via a NEB HiFi assembly reaction;
the nucleic acid sequence is introduced into the cells for expression by a vector; or
the nucleic acid sequence is introduced into the cells using a pET21b(+) expression vector.

83. A method for researching the binding activity of an immunoglobulin, comprising:
selecting a immunoglobulin;
crosslinking to the immunoglobulin a blocking construct selected from the blocking construct of any of claim 1, 2, 31-56, or 58; and
measuring the binding activity of the immunoglobulin.

84. The method of claim 81, further comprising exposing the blocking construct to a trigger to activate its cleavable linker and thereby modulate the binding activity of the immunoglobulin.

85. A kit for use in modifying the binding activity of an antigen binding domain, comprising two or more components selected from: a kappa light chain-binding polypeptide of any of claim 3-30 or 57; a blocking construct of any of claim 1, 2, 31-56, or 58; a blocked immunoglobulin complex of any of claims 59-64; and a pharmaceutical composition of claim 65 or claim 66.

86. The kit of claim 85, further comprising instructions for combining the components.


SEQUENCE LISTING (90 sequences)

SEQ ID NO: 1 - protein, 61 aa, synthetic construct
EVTIKVNLIFADGKIQTAEFKGTFEEATAEAYRYADLLAKVNGEYTADLEDGGNHMNIKFA

SEQ ID NO: 2 - protein, 61 aa, synthetic construct
EVTIKANLIFADGSTQNAEFKGTFAKAVSDAYAYADALKKDNGEYTVDVADKGLTLNIKFA

SEQ ID NO: 3 - protein, 61 aa, synthetic construct
EVTIKVNLIFADGKTQTAEFKGTFEEATAKAYAYADLLAKENGEYTADLEDGGNTINIKFA

SEQ ID NO: 4 - protein, 61 aa, synthetic construct
EVTIKVNLIFADGKIQTAEFKGTFEEATAKAYAYANLLAKENGEYTADLEDGGNTINIKFA

SEQ ID NO: 5 - protein, 61 aa, synthetic construct
EVTIKVNLIFADGKTQTAEFKGTFEEATAEAYRYADLLAKVNGEYTADLEDGGYTINIKFA

SEQ ID NO: 6 - protein, 61 aa, synthetic construct
EVTIKANLIFANGSTQTAEFKGTFEKATSEAYAYADTLKKDNGEYTVDVADKGYTLNIKFA

SEQ ID NO: 7 - protein, 61 aa, synthetic construct
EVTIKANLIYADGKTQTAEFKGTFEEATAEAYRYADALKKDNGEYTVDVADKGYTLNIKFA

SEQ ID NO: 8 - protein, 61 aa, synthetic construct
EVTIKANLIYADGKTQTAEFKGTFEEATAEAYRYADLLAKENGKYTVDVADKGYTLNIKFA

SEQ ID NO: 9 - protein, 61 aa, synthetic construct
EVTIKANLIYADGKTQTAEFKGTFAEATAEAYRYADLLAKENGKYTADLEDGGYTINIRFA

SEQ ID NO: 10 - protein, 61 aa, synthetic construct
QVTIEANIYYEDGTVQTATFKGTFAEATAEAYRYADLLSKEHGKYTADLEDGGYTINIRFA

SEQ ID NO: 11 - protein, 992 aa, synthetic construct
MKINKKLLMAALAGAIVVGGGANAYAAEEDNTDNNLSMDEISDAYFDYHGDVSDSVDPVEEEIDEALAKALAEAKETAKKHIDSLNHLSETAKKLAKNDIDSATTINAINDIVARADVMERKTAEKEEAEKLAAAKETAKKHIDELKHLADKTKELAKRDIDSATTINAINDIVARADVMERKTAEKEEAEKLAAAKETAKKHIDELKHLADKTKELAKRDIDSATTIDAINDIVARADVMERKLSEKETPEPEEEVTIKANLIFADGSTQNAEFKGTFAKAVSDAYAYADALKKDNGEYTVDVADKGLTLNIKFAGKKEKPEEPKEEVTIKVNLIFADGKTQTAEFKGTFEEATAKAYAYADLLAKENGEYTADLEDGGNTINIKFAGKETPETPEEPKEEVTIKVNLIFADGKIQTAEFKGTFEEATAKAYAYANLLAKENGEYTADLEDGGNTINIKFAGKETPETPEEPKEEVTIKVNLIFADGKTQTAEFKGTFEEATAEAYRYADLLAKVNGEYTADLEDGGYTINIKFAGKEQPGENPGITIDEWLLKNAKEEAIKELKEAGITSDLYFSLINKAKTVEGVEALKNEILKAHAGEETPELKDGYATYEEAEAAAKEALKNDDVNNAYEIVQGADGRYYYVLKIEVADEEEPGEDTPEVQEGYATYEEAEAAAKEALKEDKVNNAYEVVQGADGRYYYVLKIEDKEDEQPGEEPGENPGITIDEWLLKNAKEDAIKELKEAGISSDIYFDAINKAKTVEGVEALKNEILKAHAEKPGENPGITIDEWLLKNAKEAAIKELKEAGITAEYLFNLINKAKTVEGVESLKNEILKAHAEKPGENPGITIDEWLLKNAKEDAIKELKEAGITSDIYFDAINKAKTIEGVEALKNEILKAHKKDEEPGKKPGEDKKPEDKKPGEDKKPEDKKPGEDKKPEDKKPGKTDKDSPNKKKKAKLPKAGSEAEILTLAAAALSTAAGAYVSLKKRK

SEQ ID NO: 12 - protein, 61 aa, synthetic construct
  SITE 33: note = X is a photoreactive crosslinker residue
EVTIKVNLIFADGKIQTAEFKGTFEEATAEAYXYADLLAKVNGEYTADLEDGGNHMNIKFA

SEQ ID NO: 13 - protein, 61 aa, synthetic construct
  SITE 33: note = X is a photoreactive crosslinker residue
EVTIKANLIFADGSTQNAEFKGTFAKAVSDAYXYADALKKDNGEYTVDVADKGLTLNIKFA

SEQ ID NO: 14 - protein, 61 aa, synthetic construct
  SITE 33: note = X is a photoreactive crosslinker residue
EVTIKVNLIFADGKTQTAEFKGTFEEATAKAYXYADLLAKENGEYTADLEDGGNTINIKFA

SEQ ID NO: 15 - protein, 61 aa, synthetic construct
  SITE 33: note = X is a photoreactive crosslinker residue
EVTIKVNLIFADGKIQTAEFKGTFEEATAKAYXYANLLAKENGEYTADLEDGGNTINIKFA

SEQ ID NO: 16 - protein, 61 aa, synthetic construct
  SITE 33: note = X is a photoreactive crosslinker residue
EVTIKVNLIFADGKTQTAEFKGTFEEATAEAYXYADLLAKVNGEYTADLEDGGYTINIKFA

SEQ ID NO: 17 - protein, 61 aa, synthetic construct
  SITE 33: note = X is a photoreactive crosslinker residue
EVTIKANLIFANGSTQTAEFKGTFEKATSEAYXYADTLKKDNGEYTVDVADKGYTLNIKFA

SEQ ID NO: 18 - protein, 61 aa, synthetic construct
  SITE 33: note = X is a photoreactive crosslinker residue
EVTIKANLIYADGKTQTAEFKGTFEEATAEAYXYADALKKDNGEYTVDVADKGYTLNIKFA

SEQ ID NO: 19 - protein, 61 aa, synthetic construct
EVTIKANLIYADGKTQTAEFKGTFEEATAEAYXYADLLAKENGKYTVDVADKGYTLNIKFA

SEQ ID NO: 20 - protein, 61 aa, synthetic construct
  SITE 33: note = X is a photoreactive crosslinker residue
EVTIKANLIYADGKTQTAEFKGTFAEATAEAYXYADLLAKENGKYTADLEDGGYTINIRFA

SEQ ID NO: 21 - protein, 61 aa, synthetic construct
  SITE 33: note = X is a photoreactive crosslinker residue
QVTIEANIYYEDGTVQTATFKGTFAEATAEAYXYADLLSKEHGKYTADLEDGGYTINIRFA

SEQ ID NO: 22 - protein, 992 aa, synthetic construct
  SITE 508: note = X is a photoreactive crosslinker residue
MKINKKLLMAALAGAIVVGGGANAYAAEEDNTDNNLSMDEISDAYFDYHGDVSDSVDPVEEEIDEALAKALAEAKETAKKHIDSLNHLSETAKKLAKNDIDSATTINAINDIVARADVMERKTAEKEEAEKLAAAKETAKKHIDELKHLADKTKELAKRDIDSATTINAINDIVARADVMERKTAEKEEAEKLAAAKETAKKHIDELKHLADKTKELAKRDIDSATTIDAINDIVARADVMERKLSEKETPEPEEEVTIKANLIFADGSTQNAEFKGTFAKAVSDAYAYADALKKDNGEYTVDVADKGLTLNIKFAGKKEKPEEPKEEVTIKVNLIFADGKTQTAEFKGTFEEATAKAYAYADLLAKENGEYTADLEDGGNTINIKFAGKETPETPEEPKEEVTIKVNLIFADGKIQTAEFKGTFEEATAKAYAYANLLAKENGEYTADLEDGGNTINIKFAGKETPETPEEPKEEVTIKVNLIFADGKTQTAEFKGTFEEATAEAYXYADLLAKVNGEYTADLEDGGYTINIKFAGKEQPGENPGITIDEWLLKNAKEEAIKELKEAGITSDLYFSLINKAKTVEGVEALKNEILKAHAGEETPELKDGYATYEEAEAAAKEALKNDDVNNAYEIVQGADGRYYYVLKIEVADEEEPGEDTPEVQEGYATYEEAEAAAKEALKEDKVNNAYEVVQGADGRYYYVLKIEDKEDEQPGEEPGENPGITIDEWLLKNAKEDAIKELKEAGISSDIYFDAINKAKTVEGVEALKNEILKAHAEKPGENPGITIDEWLLKNAKEAAIKELKEAGITAEYLFNLINKAKTVEGVESLKNEILKAHAEKPGENPGITIDEWLLKNAKEDAIKELKEAGITSDIYFDAINKAKTIEGVEALKNEILKAHKKDEEPGKKPGEDKKPEDKKPGEDKKPEDKKPGEDKKPEDKKPGKTDKDSPNKKKKAKLPKAGSEAEILTLAAAALSTAAGAYVSLKKRK

SEQ ID NO: 23 - protein, 16 aa, synthetic construct
  SITE 10: note = X is a photoreactive crosslinker residue
FEEATAEAYXYADLLA

SEQ ID NO: 24 - protein, 16 aa, synthetic construct
  SITE 10: note = X is a photoreactive linker residue
FAKAVSDAYXYADALK

SEQ ID NO: 25 - protein, 16 aa, synthetic construct
  SITE 10: note = X is a photoreactive crosslinker residue
FEEATAKAYXYADLLA

SEQ ID NO: 26 - protein, 16 aa, synthetic construct
  SITE 10: note = X is a photoreactive crosslinker residue
FEEATAKAYXYANLLA

SEQ ID NO: 27 - protein, 16 aa, synthetic construct
  SITE 10: note = X is a photoreactive reactive crosslinker residue
FEKATSEAYXYADTLK

SEQ ID NO: 28 - protein, 16 aa, synthetic construct
  SITE 10: note = X is a photoreactive crosslinker residue
FEEATAEAYXYADALK

SEQ ID NO: 29 - protein, 16 aa, synthetic construct
  SITE 10: note = X is a photoreactive crosslinker residue
FAEATAEAYXYADLLA

SEQ ID NO: 30 - protein, 16 aa, synthetic construct
  SITE 10: note = X is substituted by a photoreactive crosslinker residue
FAEATAEAYXYADLLS

SEQ ID NO: 31 - protein, 8 aa, synthetic construct
DYKDDDDK

SEQ ID NO: 32 - protein, 21 aa, synthetic construct
QGQSGQCISPRGCPDGPYVMY

SEQ ID NO: 33 - protein, 5 aa, synthetic construct
LPETG

SEQ ID NO: 34 - protein, 55 aa, synthetic construct
  SITE 55: note = Linked to a Sortase A recognition site comprising the amino acid sequence Leu Pro Glu Thr Gly
GGGGSEAAAKEAAAKEAAAKEAAAKGGGGSEAAAKEAAAKEAAAKEAAAKGGGGS

SEQ ID NO: 35 - protein, 55 aa, synthetic construct; identical to SEQ ID NO: 34

SEQ ID NO: 36 - protein, 49 aa, synthetic construct
  SITE 49: note = Linked to a Sortase A recognition site comprising the amino acid sequence Leu Pro Glu Thr Gly
GGSEAAAKEAAAKEAAAKEAAAKGGSEAAAKEAAAKEAAAKEAAAKGGS

SEQ ID NO: 37 - protein, 49 aa, synthetic construct; identical to SEQ ID NO: 36

SEQ ID NO: 38 - DNA, 183 bp, synthetic construct
  source 1..183: mol_type = other DNA
gaagtaacaatcaaggtcaatttgatcttcgcggatgggaagattcagacggctgagttcaaggggacgtttgaagaagcaacggccgaggcgtaccgttacgctgatcttttggcaaaagtgaacggtgagtacactgctgacttagaggatggagggaaccacatgaacattaaattcgcg

SEQ ID NO: 39 - DNA, 528 bp, synthetic construct
  source 1..528: mol_type = other DNA
ggcctggtggcgctgacccatcgcattctggaactgtatagcgcgctggcggcgagcaacctggaaattctggaaccgcatgaagcgctggcggcgagcccgggcctgtatagcgaacgcacccatcgcggcctgaacgcgagcaacgcgctggcgggcctgccgcatgaactgtatagcggcctgtatacccatcgcccgcatgaagcgctggcgctgtatagcgcgctggcggtggcgctgagcgaacgcgcgagcccggcgctggcgacctatcgcgcgctggcgacctatcgcgcgctggcggcgagcccggcgctggcgctggaactgtatagcctgtatagcgcgagcccggcgagcaacggcctgtatggcctgacctatcgcacccatcgcgtggcgctggcgagcccggtggcgctggcgctggcggcgagcccgctgtatagcggcctgtatctggaaacccatcgcctggaagcgagcaacattctggaactgtatagcccgcatgaagcgctggcg

SEQ ID NO: 40 - DNA, 516 bp, synthetic construct
  source 1..516: mol_type = other DNA
ggcctggtggcgctgacccatcgcattctggaactgtatagcgtggcgctggcgagcaacctggaaattctggaaccgcatgaagcgctggcggcgagcccgggcctgtatctgtatagcacccatcgcggcctgaacacccatcgcgcgctggcgggcctgccgcatgaactgtatagcggcctgtatacccatcgcccgcatgaaggcctgggcctggcgctggcgacccatcgcgcgctggcgctgtatagcgcgctggcgacctatcgcgcgctggcgacctatcgcgcgctggcggcgagcccgctggaactggaagcgctggcgctgtatagcggcctggcgagcaacggcctgtatggcctgacctatcgcacccatcgcgcgctggcggcgagcccgctggaaggcctggcgagcccgggcctgtatggcctgtatgcgagcaacacccatcgcattctggaagcgagcaacattctggaactgtatagcccgcatgaagcgctggcg

SEQ ID NO: 41 - DNA, 516 bp, synthetic construct
  source 1..516: mol_type = other DNA
ggcctggtggcgctgacccatcgcattctggaactgtatagcgtggcgctggcgagcaacctggaaattctggaaccgcatgaagcgctggcggcgagcccgggcctgtatctgtatagcattctggaaggcctgaacacccatcgcgcgctggcgggcctgccgcatgaactgtatagcggcctgtatacccatcgcccgcatgaaggcctgggcctggcgctggcgacccatcgcgcgctggcgctgtatagcgcgctggcgacctatcgcgcgctggcgacctatcgcgcgctggcggcgagcaacctggaactggaagcgctggcgctgtatagcggcctggcgagcaacggcctgtatggcctgacctatcgcacccatcgcgcgctggcggcgagcccgctggaaggcctggcgagcccgggcctgtatggcctgtatgcgagcaacacccatcgcattctggaagcgagcaacattctggaactgtatagcccgcatgaagcgctggcg

SEQ ID NO: 42 - DNA, 516 bp, synthetic construct
  source 1..516: mol_type = other DNA
ggcctggtggcgctgacccatcgcattctggaactgtatagcgtggcgctggcgagcaacctggaaattctggaaccgcatgaagcgctggcggcgagcccgggcctgtatctgtatagcacccatcgcggcctgaacacccatcgcgcgctggcgggcctgccgcatgaactgtatagcggcctgtatacccatcgcccgcatgaaggcctgggcctggcgctggcgacccatcgcgcgctggcgggcctggcgctggcgacctatcgcgcgcgcggcacctatcgcgcgctggcggcgagcccgctggaactggaagcgctggcgctgtatagcgtggcgctggcgagcaacggcctgtatggcctgacctatcgcacccatcgcgcgctggcggcgagcccgctggaaggcctggcgagcccgggcctgtatggcctgtatacctatcgcacccatcgcattctggaagcgagcaacattctggaactgtatagcccgcatgaagcgctggcg

SEQ ID NO: 43 - DNA, 525 bp, synthetic construct
  source 1..525: mol_type = other DNA
ggcctggtggcgctgacccatcgcattctggaactgtatagcgcgctggcggcgagcaacctggaaattctggaaccgcatgaagcgctggcggcgagcaacggcctgtatagcgaacgcacccatcgcggcctgaacacccatcgcgcgctggcgggcctgccgcatgaactgtatagcggcctgtatacccatcgcccgcatgaaggcctgctgtatagcgcgctggcgacccatcgcagcgaacgcggcctggcgctggcgacctatcgcgcgctggcgacctatcgcgcgctggcggcgagcccgacccatcgcctggaactgtatagcctgtatagcgcgagcccggcgagcaacggcctgtatggcctgacctatcgcacccatcgcgtggcgctggcgagcccggtggcgctggcgctggcggcgagcccgctgtatagcggcctgtatacctatcgcacccatcgcctggaagcgagcaacattctggaactgtatagcccgcatgaagcgctggcg

SEQ ID NO: 44 - DNA, 522 bp, synthetic construct
  source 1..522: mol_type = other DNA
ggcctggtggcgctgacccatcgcattctggaactgtatagcgcgctggcggcgagcaacctggaaattctggaaacctatcgcgcgctggcggcgagcccgggcctgtatctgtatagcacccatcgcggcctgaacacccatcgcgcgctggcgggcctgccgcatgaactgtatagcggcctgtatacccatcgcccgcatgaaggcctgggcctggcgctggcgacccatcgcgcgctggcgggcctggcgctggcgacctatcgcgcgcgcggcacctatcgcgcgctggcggcgagcccggcgctggcgctggaactgtatagcctgtatagcgcgagcccggcgagcaacggcctgtatggcctgacctatcgcacccatcgcgtggcgctggcgagcccggtggcgctggcgctggcggcgagcccgctgtatagcggcctgtatacctatcgcacccatcgcctggaagcgagcaacattctggaactgtatagcccgcatgaagcgctggcg

SEQ ID NO: 45 - DNA, 519 bp, synthetic construct
  source 1..519: mol_type = other DNA
ggcctggtggcgctgacccatcgcattctggaactgtatagcgcgctggcggcgagcaacctggaaattctggaaacctatcgcgcgctggcggcgagcccgggcctgtatctgtatagcacccatcgcggcctgaacacccatcgcgcgctggcgggcctgccgcatgaactgtatagcggcctgtatacccatcgcccgcatgaaggcctgggcctggcgctggcgacccatcgcgcgctggcgggcctggcgctggcgacctatcgcgcgcgcggcacctatcgcgcgctggcggcgagcccgctggaactggaagcgctggcgctgtatagcggcctggcgagcaacggcctgtatctgtatagcacctatcgcacccatcgcgtggcgctggcgagcccggtggcgctggcgctggcggcgagcccgctgtatagcggcctgtatacctatcgcacccatcgcctggaagcgagcaacattctggaactgtatagcccgcatgaagcgctggcg

SEQ ID NO: 46 - DNA, 519 bp, synthetic construct
  source 1..519: mol_type = other DNA
ggcctggtggcgctgacccatcgcattctggaactgtatagcgcgctggcggcgagcaacctggaaattctggaaacctatcgcgcgctggcggcgagcccgggcctgtatctgtatagcacccatcgcggcctgaacacccatcgcgcgctggcgggcctgccgcatgaactgtatagcggcctgtatacccatcgcccgcatgaagcgctggcgggcctggcgctggcgacccatcgcgcgctggcgggcctggcgctggcgacctatcgcgcgcgcggcacctatcgcgcgctggcggcgagcccgctggaactggaagcgctggcgctgtatagcggcctggcgagcaacggcctgtatctgtatagcacctatcgcacccatcgcgcgctggcggcgagcccgctggaaggcctggcgagcccgggcctgtatggcctgtatacctatcgcacccatcgcattctggaagcgagcaacattctggaagcgcgcggcccgcatgaagcgctggcg

SEQ ID NO: 47 - DNA, 522 bp, synthetic construct
  source 1..522: mol_type = other DNA
ggcctgaacgtggcgctgacccatcgcattctggaaggcctggcgctggcggcgagcaacattctggaaacctatcgcacctatcgcggcctggcgagcccgggcctgtatacccatcgcgtggcgctgggcctgaacacccatcgcgcgctggcgacccatcgcccgcatgaactgtatagcggcctgtatacccatcgcccgcatgaagcgctggcgggcctggcgctggcgacccatcgcgcgctggcgggcctggcgctggcgacctatcgcgcgcgcggcacctatcgcgcgctggcggcgagcccgctggaactggaaagcgaacgcctgtatagcggcctgcatattagcggcctgtatctgtatagcacctatcgcacccatcgcgcgctggcggcgagcccgctggaaggcctggcgagcccgggcctgtatggcctgtatacctatcgcacccatcgcattctggaagcgagcaacattctggaagcgcgcggcccgcatgaagcgctggcg

SEQ ID NO: 48 - DNA, 8196 bp, synthetic construct
  source 1..8196: mol_type = other DNA
atggaaaccctgtatagcattctggaagcgagcaacctgtatagcctgtatagcctggaactggaaatggaaaccgcgctggcggcgctggcgctggaagcgctggcgggcctgtatgcgctggcgattctggaagtggcgctggtggcgctgggcctgtatggcctgtatggcctgtatgcgctggcggcgagcaacgcgctggcgacctatcgcgcgctggcggcgctggcgggcctgggcctggcgagcccggcgagcaacacccatcgcgcgagcccggcgagcaacgcgagcaacctggaaagcgaacgcatggaaaccgcgagcccgggcctgattctggaaagcgaacgcgcgagcccggcgctggcgacctatcgcccgcatgaagcgagcccgacctatcgccatattagcggcctgtatgcgagcccggtggcgctgagcgaacgcgcgagcccgagcgaacgcgtggcgctggcgagcccgccgcgcgtggcgctgggcctgggcctgggcctgattctggaagcgagcccgggcctggcgctggcgctggaagcgctggcgctgtatagcgcgctggcgctggaagcgctggcgggcctggcgctggcgctgtatagcggcctgacccatcgcgcgctggcgctgtatagcctgtatagccatattagcattctggaagcgagcccgagcgaacgcctggaagcgagcaaccatattagcctggaaagcgaacgcggcctgacccatcgcgcgctggcgctgtatagcctgtatagcctggaagcgctggcgctgtatagcgcgagcaacgcgagcccgattctggaagcgagcccgagcgaacgcgcgctggcgacccatcgcacccatcgcattctggaagcgagcaacgcgctggcgattctggaagcgagcaacgcgagcccgattctggaagtggcgctggcgctggcggcgcgcggcgcgctggcggcgagcccggtggcgctgatggaaaccggcctggcgcgcggcctgtatagcacccatcgcgcgctggcgggcctgctgtatagcggcctgggcctggcgctggcgggcctgctgtatagcctggaagcgctggcggcgctggcggcgctggcgctgtatagcggcctgacccatcgcgcgctggcgctgtatagcctgtatagccatattagcattctggaagcgagcccgggcctgctggaactgtatagccatattagcctggaagcgctggcggcgagcccgctgtatagcacccatcgcctgtatagcggcctgctggaagcgctggcgctgtatagcgcgcgcggcgcgagcccgattctggaagcgagcccgagcgaacgcgcgctggcgacccatcgcacccatcgcattctggaagcgagcaacgcgctggcgattctggaagcgagcaacgcgagcccgattctggaagtggcgctggcgctggcggcgcgcggcgcgctggcggcgagcccggtggcgctgatggaaaccggcctggcgcgcggcctgtatagcacccatcgcgcgctggcgggcctgctgtatagcggcctgggcctggcgctggcgggcctgctgtatagcctggaagcgctggcggcgctggcggcgctggcgctgtatagcggcctgacccatcgcgcgctggcgctgtatagcctgtatagccatattagcattctggaagcgagcccgggcctgctggaactgtatagccatattagcctggaagcgctggcggcgagcccgctgtatagcacccatcgcctgtatagcggcctgctggaagcgctggcgctgtatagcgcgcgcggcgcgagcccgattctggaagcgagcccgagcgaacgcgcgctggcgacccatcgcacccatcgcattctggaagcgagcccggcgctggcgattctggaagcgagcaacgcgagcccgattctggaagtggcgctggcgctggcggcgcgcggcgcgctggcggcgagcccggtggcgctgatggaaaccggcctggcgcgcggcctgtatagcctggaaagcgaacgcggcctgctgtatagcggcctgacccatcgcccgcgcggcctgccgcgcggcctgggcctgggcctggtggcgctgacccatcgcattctggaactgtatagcgcgctggcggcgagcaacctggaaattctggaaccgcatgaagcgctggcggcgagcccgggcctgtatagcgaacgcacccatcgcggcctgaacgcgagcaacgcgctggcgggcctgccgcatgaactgtatagcggcctgtatacccatcgcccgcatgaagcgctggcgctgtatagcgcgctggcggtggcgctgagcgaacgcgcgagcccggcgctggcgacctatcgcgcgctggcgacctatcgcgcgctggcggcgagcccggcgctggcgctggaactgtatagcctgtatagcgcgagcccggcgagcaacggcctgtatggcctgacctatcgcacccatcgcgtggcgctggcgagcccggtggcgctggcgctggcggcgagcccgctgtatagcggcctgtatctggaaacccatcgcctggaagcgagcaacattctggaactgtatagcccgcatgaagcgctggcgggcctgtatctgtatagcctgtatagcggcctgctgtatagcccgcgcggcctgggcctgccgcgcctgtatagcggcctgggcctggtggcgctgacccatcgcattctggaactgtatagcgtggcgctggcgagcaacctggaaattctggaaccgcatgaagcgctggcggcgagcccgggcctgtatctgtatagcacccatcgcggcctgaacacccatcgcgcgctggcgggcctgccgcatgaactgtatagcggcctgtatacccatcgcccgcatgaaggcctgggcctggcgctggcgacccatcgcgcgctggcgctgtatagcgcgctggcgacctatcgcgcgctggcgacctatcgcgcgctggcggcgagcccgctggaactggaagcgctggcgctgtatagcggcctggcgagcaacggcctgtatggcctgacctatcgcacccatcgcgcgctggcggcgagcccgctggaaggcctggcgagcccgggcctgtatggcctgtatgcgagcaacacccatcgcattctggaagcgagcaacattctggaactgtatagcccgcatgaagcgctggcgggcctgtatctgtatagcggcctgacccatcgcccgcgcggcctgacccatcgcccgcgcggcctgggcctgccgcgcctgtatagcggcctgggcctggtggcgctgacccatcgcattctggaactgtatagcgtggcgctggcgagcaacctggaaattctggaaccgcatgaagcgctggcggcgagcccgggcctgtatctgtatagcattctggaaggcctgaacacccatcgcgcgctggcgggcctgccgcatgaactgtatagcggcctgtatacccatcgcccgcatgaaggcctgggcctggcgctggcgacccatcgcgcgctggcgctgtatagcgcgctggcgacctatcgcgcgctggcgacctatcgcgcgctggcggcgagcaacctggaactggaagcgctggcgctgtatagcggcctggcgagcaacggcctgtatggcctgacctatcgcacccatcgcgcgctggcggcgagcccgctggaaggcctggcgagcccgggcctgtatggcctgtatgcgagcaacacccatcgcattctggaagcgagcaacattctggaactgtatagcccgcatgaagcgctggcgggcctgtatctgtatagcggcctgacccatcgcccgcgcggcctgacccatcgcccgcgcggcctgggcctgccgcgcctgtatagcggcctgggcctggtggcgctgacccatcgcattctggaactgtatagcgtggcgctggcgagcaacctggaaattctggaaccgcatgaagcgctggcggcgagcccgggcctgtatctgtatagcacccatcgcggcctgaacacccatcgcgcgctggcgggcctgccgcatgaactgtatagcggcctgtatacccatcgcccgcatgaaggcctgggcctggcgctggcgacccatcgcgcgctggcgggcctggcgctggcgacctatcgcgcgcgcggcacctatcgcgcgctggcggcgagcccgctggaactggaagcgctggcgctgtatagcgtggcgctggcgagcaacggcctgtatggcctgacctatcgcacccatcgcgcgctggcggcgagcccgctggaaggcctggcgagcccgggcctgtatggcctgtatacctatcgcacccatcgcattctggaagcgagcaacattctggaactgtatagcccgcatgaagcgctggcgggcctgtatctgtatagcggcctgggcctgaacccgcgcggcctgtatggcctggcgagcaacccgcgcggcctgtatattctggaaacccatcgcattctggaagcgagcccgggcctgacccgcccgctggaactggaactgtatagcgcgagcaacgcgctggcgctgtatagcggcctgggcctggcgctggcgattctggaactgtatagcggcctgctggaactgtatagcggcctggcgctggcgggcctgtatattctggaaacccatcgcagcgaacgcgcgagcccgctggaaacctatcgcccgcatgaaagcgaacgcctggaaattctggaagcgagcaacctgtatagcgcgctggcgctgtatagcacccatcgcgtggcgctgggcctgggcctgtatgtggcgctgggcctggcgctggcgctggaactgtatagcgcgagcaacggcctgattctggaactggaactgtatagcgcgctggcgcatattagcgcgctggcgggcctgtatggcctgggcctgacccatcgcccgcgcggcctgctggaactgtatagcgcgagcccgggcctgtatacctatcgcgcgctggcgacccatcgcacctatcgcggcctgggcctggcgctggcgggcctggcgctggcggcgctggcggcgctggcgctgtatagcggcctggcgctggcgctggaactgtatagcgcgagcaacgcgagcccggcgagcccggtggcgctggcgagcaacgcgagcaacgcgctggcgacctatcgcggcctgattctggaagtggcgctgggcctgaacggcctgtatgcgctggcggcgagcccgggcctgtatgcgcgcggcacctatcgcacctatcgcacctatcgcgtggcgctgctggaactgtatagcattctggaaggcctggtggcgctggcgctggcggcgagcccgggcctgggcctgggcctgccgcgcggcctgtatggcctggcgagcccgacccatcgcccgcgcggcctggtggcgctgggcctgaacggcctgggcctgtatacctatcgcgcgctggcgacccatcgcacctatcgcggcctgggcctggcgctggcgggcctggcgctggcggcgctggcggcgctggcgctgtatagcggcctggcgctggcgctggaactgtatagcggcctggcgagcccgctgtatagcgtggcgctggcgagcaacgcgagcaacgcgctggcgacctatcgcggcctggtggcgctggtggcgctgggcctgaacggcctgtatgcgctggcggcgagcccgggcctgtatgcgcgcggcacctatcgcacctatcgcacctatcgcgtggcgctgctggaactgtatagcattctggaaggcctggcgagcccgctgtatagcggcctggcgagcccgggcctgggcctgaacccgcgcggcctgtatggcctgggcctgccgcgcggcctgtatggcctggcgagcaacccgcgcggcctgtatattctggaaacccatcgcattctggaagcgagcccgggcctgacccgcccgctggaactggaactgtatagcgcgagcaacgcgctggcgctgtatagcggcctggcgagcccggcgctggcgattctggaactgtatagcggcctgctggaactgtatagcggcctggcgctggcgggcctgtatattctggaaagcgaacgcagcgaacgcgcgagcccgattctggaaacctatcgcccgcatgaagcgagcccggcgctggcgattctggaagcgagcaacctgtatagcgcgctggcgctgtatagcacccatcgcgtggcgctgggcctgggcctgtatgtggcgctgggcctggcgctggcgctggaactgtatagcgcgagcaacggcctgattctggaactggaactgtatagcgcgctggcgcatattagcgcgctggcgggcctgctgtatagcccgcgcggcctgtatggcctggcgagcaacccgcgcggcctgtatattctggaaacccatcgcattctggaagcgagcccgggcctgacccgcccgctggaactggaactgtatagcgcgagcaacgcgctggcgctgtatagcggcctggcgctggcggcgctggcgattctggaactgtatagcggcctgctggaactgtatagcggcctggcgctggcgggcctgtatattctggaaacccatcgcgcgctggcgggcctgacctatcgcctggaaccgcatgaagcgagcaacctggaaattctggaagcgagcaacctgtatagcgcgctggcgctgtatagcacccatcgcgtggcgctgggcctgggcctgtatgtggcgctgggcctgagcgaacgcctggaactgtatagcgcgagcaacggcctgattctggaactggaactgtatagcgcgctggcgcatattagcgcgctggcgggcctgctgtatagcccgcgcggcctgtatggcctggcgagcaacccgcgcggcctgtatattctggaaacccatcgcattctggaagcgagcccgggcctgacccgcccgctggaactggaactgtatagcgcgagcaacgcgctggcgctgtatagcggcctggcgagcccggcgctggcgattctggaactgtatagcggcctgctggaactgtatagcggcctggcgctggcgggcctgtatattctggaaacccatcgcagcgaacgcgcgagcccgattctggaaacctatcgcccgcatgaagcgagcccggcgctggcgattctggaagcgagcaacctgtatagcgcgctggcgctgtatagcacccatcgcattctggaaggcctgggcctgtatgtggcgctgggcctggcgctggcgctggaactgtatagcgcgagcaacggcctgattctggaactggaactgtatagcgcgctggcgcatattagcctgtatagcctgtatagcgcgagcccgggcctgggcctgccgcgcggcctgtatctgtatagcctgtatagcccgcgcggcctgtatggcctggcgagcccgctgtatagcctgtatagcccgcgcggcctggcgagcccgctgtatagcctgtatagcccgcgcggcctgtatggcctggcgagcccgctgtatagcctgtatagcccgcgcggcctggcgagcccgctgtatagcctgtatagcccgcgcggcctgtatggcctggcgagcccgctgtatagcctgtatagcccgcgcggcctggcgagcccgctgtatagcctgtatagcccgcgcggcctgtatctgtatagcacccatcgcgcgagcccgctgtatagcgcgagcccgagcgaacgcccgcgcgcgagcaacctgtatagcctgtatagcctgtatagcctgtatagcgcgctggcgctgtatagcctggaaccgcgcctgtatagcgcgctggcgggcctgtatagcgaacgcggcctggcgctggcgggcctgattctggaactggaaacccatcgcctggaagcgctggcggcgctggcggcgctggcggcgctggcgctggaaagcgaacgcacccatcgcgcgctggcggcgctggcgggcctgtatgcgctggcgacctatcgcgtggcgctgagcgaacgcctggaactgtatagcctgtatagcgcgcgcggcctgtatagc

SEQ ID NO: 49 - DNA, 516 bp, synthetic construct
  source 1..516: mol_type = other DNA
  misc_feature 271..273: note = NNN is a codon encoding a photoreactive crosslinker residue
ggcctggtggcgctgacccatcgcattctggaactgtatagcgtggcgctggcgagcaacctggaaattctggaaccgcatgaagcgctggcggcgagcccgggcctgtatctgtatagcattctggaaggcctgaacacccatcgcgcgctggcgggcctgccgcatgaactgtatagcggcctgtatacccatcgcccgcatgaaggcctgggcctggcgctggcgacccatcgcgcgctggcgggcctggcgctggcgacctatcgcnnngcggcgacctatcgcgcgctggcggcgagcccgctggaactggaagcgctggcgctgtatagcgtggcgctggcgagcaacggcctgtatggcctgacctatcgcacccatcgcgcgctggcggcgagcccgctggaaggcctggcgagcccgggcctgtatggcctgtatgcgagcaaccatattagcatggaaaccgcgagcaacattctggaactgtatagcccgcatgaagcgctggcg

SEQ ID NO: 50 - DNA, 528 bp, synthetic construct
  source 1..528: mol_type = other DNA
  misc_feature 280..282: note = NNN is a codon encoding a photo reactive crosslinker residue
ggcctggtggcgctgacccatcgcattctggaactgtatagcgcgctggcggcgagcaacctggaaattctggaaccgcatgaagcgctggcggcgagcccgggcctgtatagcgaacgcacccatcgcggcctgaacgcgagcaacgcgctggcgggcctgccgcatgaactgtatagcggcctgtatacccatcgcccgcatgaagcgctggcgctgtatagcgcgctggcggtggcgctgagcgaacgcgcgagcccggcgctggcgacctatcgcnnngcggcgacctatcgcgcgctggcggcgagcccggcgctggcgctggaactgtatagcctgtatagcgcgagcccggcgagcaacggcctgtatggcctgacctatcgcacccatcgcgtggcgctggcgagcccggtggcgctggcgctggcggcgagcccgctgtatagcggcctgtatctggaaacccatcgcctggaagcgagcaacattctggaactgtatagcccgcatgaagcgctggcg

SEQ ID NO: 51 - DNA, 516 bp, synthetic construct
  source 1..516: mol_type = other DNA
  misc_feature 274..276: note = NNN is a codon encoding a photoreactive crosslinker residue
ggcctggtggcgctgacccatcgcattctggaactgtatagcgtggcgctggcgagcaacctggaaattctggaaccgcatgaagcgctggcggcgagcccgggcctgtatctgtatagcacccatcgcggcctgaacacccatcgcgcgctggcgggcctgccgcatgaactgtatagcggcctgtatacccatcgcccgcatgaaggcctgggcctggcgctggcgacccatcgcgcgctggcgctgtatagcgcgctggcgacctatcgcnnngcggcgacctatcgcgcgctggcggcgagcccgctggaactggaagcgctggcgctgtatagcggcctggcgagcaacggcctgtatggcctgacctatcgcacccatcgcgcgctggcggcgagcccgctggaaggcctggcgagcccgggcctgtatggcctgtatgcgagcaacacccatcgcattctggaagcgagcaacattctggaactgtatagcccgcatgaagcgctggcg

SEQ ID NO: 52 - DNA, 516 bp, synthetic construct
  source 1..516: mol_type = other DNA
  misc_feature 274..276: note = NNN is a codon encoding a photoreactive crosslinker residue
ggcctggtggcgctgacccatcgcattctggaactgtatagcgtggcgctggcgagcaacctggaaattctggaaccgcatgaagcgctggcggcgagcccgggcctgtatctgtatagcattctggaaggcctgaacacccatcgcgcgctggcgggcctgccgcatgaactgtatagcggcctgtatacccatcgcccgcatgaaggcctgggcctggcgctggcgacccatcgcgcgctggcgctgtatagcgcgctggcgacctatcgcnnngcggcgacctatcgcgcgctggcggcgagcaacctggaactggaagcgctggcgctgtatagcggcctggcgagcaacggcctgtatggcctgacctatcgcacccatcgcgcgctggcggcgagcccgctggaaggcctggcgagcccgggcctgtatggcctgtatgcgagcaacacccatcgcattctggaagcgagcaacattctggaactgtatagcccgcatgaagcgctggcg

SEQ ID NO: 53 - DNA, 516 bp, synthetic construct
  source 1..516: mol_type = other DNA
  misc_feature 271..273: note = NNN is a codon encoding a photoreactive crosslinker residue
ggcctggtggcgctgacccatcgcattctggaactgtatagcgtggcgctggcgagcaacctggaaattctggaaccgcatgaagcgctggcggcgagcccgggcctgtatctgtatagcacccatcgcggcctgaacacccatcgcgcgctggcgggcctgccgcatgaactgtatagcggcctgtatacccatcgcccgcatgaaggcctgggcctggcgctggcgacccatcgcgcgctggcgggcctggcgctggcgacctatcgcnnngcggcgacctatcgcgcgctggcggcgagcccgctggaactggaagcgctggcgctgtatagcgtggcgctggcgagcaacggcctgtatggcctgacctatcgcacccatcgcgcgctggcggcgagcccgctggaaggcctggcgagcccgggcctgtatggcctgtatacctatcgcacccatcgcattctggaagcgagcaacattctggaactgtatagcccgcatgaagcgctggcg

SEQ ID NO: 54 - DNA, 525 bp, synthetic construct
  source 1..525: mol_type = other DNA
  misc_feature 274..276: note = NNN is a codon encoding a photoreactive crosslinker residue
ggcctggtggcgctgacccatcgcattctggaactgtatagcgcgctggcggcgagcaacctggaaattctggaaccgcatgaagcgctggcggcgagcaacggcctgtatagcgaacgcacccatcgcggcctgaacacccatcgcgcgctggcgggcctgccgcatgaactgtatagcggcctgtatacccatcgcccgcatgaaggcctgctgtatagcgcgctggcgacccatcgcagcgaacgcggcctggcgctggcgacctatcgcnnngcggcgacctatcgcgcgctggcggcgagcccgacccatcgcctggaactgtatagcctgtatagcgcgagcccggcgagcaacggcctgtatggcctgacctatcgcacccatcgcgtggcgctggcgagcccggtggcgctggcgctggcggcgagcccgctgtatagcggcctgtatacctatcgcacccatcgcctggaagcgagcaacattctggaactgtatagcccgcatgaagcgctggcg

SEQ ID NO: 55 - DNA, 522 bp, synthetic construct
  source 1..522: mol_type = other DNA
  misc_feature 271..273: note = NNN is a codon encoding a photoreactive crosslinker residue
ggcctggtggcgctgacccatcgcattctggaactgtatagcgcgctggcggcgagcaacctggaaattctggaaacctatcgcgcgctggcggcgagcccgggcctgtatctgtatagcacccatcgcggcctgaacacccatcgcgcgctggcgggcctgccgcatgaactgtatagcggcctgtatacccatcgcccgcatgaaggcctgggcctggcgctggcgacccatcgcgcgctggcgggcctggcgctggcgacctatcgcnnngcggcgacctatcgcgcgctggcggcgagcccggcgctggcgctggaactgtatagcctgtatagcgcgagcccggcgagcaacggcctgtatggcctgacctatcgcacccatcgcgtggcgctggcgagcccggtggcgctggcgctggcggcgagcccgctgtatagcggcctgtatacctatcgcacccatcgcctggaagcgagcaacattctggaactgtatagcccgcatgaagcgctggcg

SEQ ID NO: 56 - DNA, 519 bp, synthetic construct
  source 1..519: mol_type = other DNA
  misc_feature 271..273: note = NNN is a codon encoding a photoreactive crosslinker residue
ggcctggtggcgctgacccatcgcattctggaactgtatagcgcgctggcggcgagcaacctggaaattctggaaacctatcgcgcgctggcggcgagcccgggcctgtatctgtatagcacccatcgcggcctgaacacccatcgcgcgctggcgggcctgccgcatgaactgtatagcggcctgtatacccatcgcccgcatgaaggcctgggcctggcgctggcgacccatcgcgcgctggcgggcctggcgctggcgacctatcgcnnngcggcgacctatcgcgcgctggcggcgagcccgctggaactggaagcgctggcgctgtatagcggcctggcgagcaacggcctgtatctgtatagcacctatcgcacccatcgcgtggcgctggcgagcccggtggcgctggcgctggcggcgagcccgctgtatagcggcctgtatacctatcgcacccatcgcctggaagcgagcaacattctggaactgtatagcccgcatgaagcgctggcg

SEQ ID NO: 57 - DNA, 519 bp, synthetic construct
  source 1..519: mol_type = other DNA
  misc_feature 274..276: note = NNN is a codon encoding a photoreactive crosslinker residue
ggcctggtggcgctgacccatcgcattctggaactgtatagcgcgctggcggcgagcaacctggaaattctggaaacctatcgcgcgctggcggcgagcccgggcctgtatctgtatagcacccatcgcggcctgaacacccatcgcgcgctggcgggcctgccgcatgaactgtatagcggcctgtatacccatcgcccgcatgaagcgctggcgggcctggcgctggcgacccatcgcgcgctggcgggcctggcgctggcgacctatcgcnnngcggcgacctatcgcgcgctggcggcgagcccgctggaactggaagcgctggcgctgtatagcggcctggcgagcaacggcctgtatctgtatagcacctatcgcacccatcgcgcgctggcggcgagcccgctggaaggcctggcgagcccgggcctgtatggcctgtatacctatcgcacccatcgcattctggaagcgagcaacattctggaagcgcgcggcccgcatgaagcgctggcg

SEQ ID NO: 58 - DNA, 522 bp, synthetic construct
  source 1..522: mol_type = other DNA
  misc_feature 277..279: note = NNN is a codon encoding a photoreactive crosslinker residue
ggcctgaacgtggcgctgacccatcgcattctggaaggcctggcgctggcggcgagcaacattctggaaacctatcgcacctatcgcggcctggcgagcccgggcctgtatacccatcgcgtggcgctgggcctgaacacccatcgcgcgctggcgacccatcgcccgcatgaactgtatagcggcctgtatacccatcgcccgcatgaagcgctggcgggcctggcgctggcgacccatcgcgcgctggcgggcctggcgctggcgacctatcgcnnngcggcgacctatcgcgcgctggcggcgagcccgctggaactggaaagcgaacgcctgtatagcggcctgcatattagcggcctgtatctgtatagcacctatcgcacccatcgcgcgctggcggcgagcccgctggaaggcctggcgagcccgggcctgtatggcctgtatacctatcgcacccatcgcattctggaagcgagcaacattctggaagcgcgcggcccgcatgaagcgctggcg

SEQ ID NO: 59 - DNA, 8196 bp, synthetic construct
  source 1..8196: mol_type = other DNA
  misc_feature 4234..4236: note = NNN is a codon encoding a photoreactive crosslinker residue
atggaaaccctgtatagcattctggaagcgagcaacctgtatagcctgtatagcctggaactggaaatggaaaccgcgctggcggcgctggcgctggaagcgctggcgggcctgtatgcgctggcgattctggaagtggcgctggtggcgctgggcctgtatggcctgtatggcctgtatgcgctggcggcgagcaacgcgctggcgacctatcgcgcgctggcggcgctggcgggcctgggcctggcgagcccggcgagcaacacccatcgcgcgagcccggcgagcaacgcgagcaacctggaaagcgaacgcatggaaaccgcgagcccgggcctgattctggaaagcgaacgcgcgagcccggcgctggcgacctatcgcccgcatgaagcgagcccgacctatcgccatattagcggcctgtatgcgagcccggtggcgctgagcgaacgcgcgagcccgagcgaacgcgtggcgctggcgagcccgccgcgcgtggcgctgggcctgggcctgggcctgattctggaagcgagcccgggcctggcgctggcgctggaagcgctggcgctgtatagcgcgctggcgctggaagcgctggcgggcctggcgctggcgctgtatagcggcctgacccatcgcgcgctggcgctgtatagcctgtatagccatattagcattctggaagcgagcccgagcgaacgcctggaagcgagcaaccatattagcctggaaagcgaacgcggcctgacccatcgcgcgctggcgctgtatagcctgtatagcctggaagcgctggcgctgtatagcgcgagcaacgcgagcccgattctggaagcgagcccgagcgaacgcgcgctggcgacccatcgcacccatcgcattctggaagcgagcaacgcgctggcgattctggaagcgagcaacgcgagcccgattctggaagtggcgctggcgctggcggcgcgcggcgcgctggcggcgagcccggtggcgctgatggaaaccggcctggcgcgcggcctgtatagcacccatcgcgcgctggcgggcctgctgtatagcggcctgggcctggcgctggcgggcctgctgtatagcctggaagcgctggcggcgctggcggcgctggcgctgtatagcggcctgacccatcgcgcgctggcgctgtatagcctgtatagccatattagcattctggaagcgagcccgggcctgctggaactgtatagccatattagcctggaagcgctggcggcgagcccgctgtatagcacccatcgcctgtatagcggcctgctggaagcgctggcgctgtatagcgcgcgcggcgcgagcccgattctggaagcgagcccgagcgaacgcgcgctggcgacccatcgcacccatcgcattctggaagcgagcaacgcgctggcgattctggaagcgagcaacgcgagcccgattctggaagtggcgctggcgctggcggcgcgcggcgcgctggcggcgagcccggtggcgctgatggaaaccggcctggcgcgcggcctgtatagcacccatcgcgcgctggcgggcctgctgtatagcggcctgggcctggcgctggcgggcctgctgtatagcctggaagcgctggcggcgctggcggcgctggcgctgtatagcggcctgacccatcgcgcgctggcgctgtatagcctgtatagccatattagcattctggaagcgagcccgggcctgctggaactgtatagccatattagcctggaagcgctggcggcgagcccgctgtatagcacccatcgcctgtatagcggcctgctggaagcgctggcgctgtatagcgcgcgcggcgcgagcccgattctggaagcgagcccgagcgaacgcgcgctggcgacccatcgcacccatcgcattctggaagcgagcccggcgctggcgattctggaagcgagcaacgcgagcccgattctggaagtggcgctggcgctggcggcgcgcggcgcgctggcggcgagcccggtggcgctgatggaaaccggcctggcgcgcggcctgtatagcctggaaagcgaacgcggcctgctgtatagcggcctgacccatcgcccgcgcggcctgccgcgcggcctgggcctgggcctggtggcgctgacccatcgcattctggaactgtatagcgcgctggcggcgagcaacctggaaattctggaaccgcatgaagcgctggcggcgagcccgggcctgtatagcgaacgcacccatcgcggcctgaacgcgagcaacgcgctggcgggcctgccgcatgaactgtatagcggcctgtatacccatcgcccgcatgaagcgctggcgctgtatagcgcgctggcggtggcgctgagcgaacgcgcgagcccggcgctggcgacctatcgcgcgctggcgacctatcgcgcgctggcggcgagcccggcgctggcgctggaactgtatagcctgtatagcgcgagcccggcgagcaacggcctgtatggcctgacctatcgcacccatcgcgtggcgctggcgagcccggtggcgctggcgctggcggcgagcccgctgtatagcggcctgtatctggaaacccatcgcctggaagcgagcaacattctggaactgtatagcccgcatgaagcgctggcgggcctgtatctgtatagcctgtatagcggcctgctgtatagcccgcgcggcctgggcctgccgcgcctgtatagcggcctgggcctggtggcgctgacccatcgcattctggaactgtatagcgtggcgctggcgagcaacctggaaattctggaaccgcatgaagcgctggcggcgagcccgggcctgtatctgtatagcacccatcgcggcctgaacacccatcgcgcgctggcgggcctgccgcatgaactgtatagcggcctgtatacccatcgcccgcatgaaggcctgggcctggcgctggcgacccatcgcgcgctggcgctgtatagcgcgctggcgacctatcgcgcgctggcgacctatcgcgcgctggcggcgagcccgctggaactggaagcgctggcgctgtatagcggcctggcgagcaacggcctgtatggcctgacctatcgcacccatcgcgcgctggcggcgagcccgctggaaggcctggcgagcccgggcctgtatggcctgtatgcgagcaacacccatcgcattctggaagcgagcaacattctggaactgtatagcccgcatgaagcgctggcgggcctgtatctgtatagcggcctgacccatcgcccgcgcggcctgacccatcgcccgcgcggcctgggcctgccgcgcctgtatagcggcctgggcctggtggcgctgacccatcgcattctggaactgtatagcgtggcgctggcgagcaacctggaaattctggaaccgcatgaagcgctggcggcgagcccgggcctgtatctgtatagcattctggaaggcctgaacacccatcgcgcgctggcgggcctgccgcatgaactgtatagcggcctgtatacccatcgcccgcatgaaggcctgggcctggcgctggcgacccatcgcgcgctggcgctgtatagcgcgctggcgacctatcgcgcgctggcgacctatcgcgcgctggcggcgagcaacctggaactggaagcgctggcgctgtatagcggcctggcgagcaacggcctgtatggcctgacctatcgcacccatcgcgcgctggcggcgagcccgctggaaggcctggcgagcccgggcctgtatggcctgtatgcgagcaacacccatcgcattctggaagcgagcaacattctggaactgtatagcccgcatgaagcgctggcgggcctgtatctgtatagcggcctgacccatcgcccgcgcggcctgacccatcgcccgcgcggcctgggcctgccgcgcctgtatagcggcctgggcctggtggcgctgacccatcgcattctggaactgtatagcgtggcgctggcgagcaacctggaaattctggaaccgcatgaagcgctggcggcgagcccgggcctgtatctgtatagcacccatcgcggcctgaacacccatcgcgcgctggcgggcctgccgcatgaactgtatagcggcctgtatacccatcgcccgcatgaaggcctgggcctggcgctggcgacccatcgcgcgctggcgggcctggcgctggcgacctatcgcnnngcggcgacctatcgcgcgctggcggcgagcccgctggaactggaagcgctggcgctgtatagcgtggcgctggcgagcaacggcctgtatggcctgacctatcgcacccatcgcgcgctggcggcgagcccgctggaaggcctggcgagcccgggcctgtatggcctgtatacctatcgcacccatcgcattctggaagcgagcaacattctggaactgtatagcccgcatgaagcgctggcgggcctgtatctgtatagcggcctgggcctgaacccgcgcggcctgtatggcctggcgagcaacccgcgcggcctgtatattctggaaacccatcgcattctggaagcgagcccgggcctgacccgcccgctggaactggaactgtatagcgcgagcaacgcgctggcgctgtatagcggcctgggcctggcgctggcgattctggaactgtatagcggcctgctggaactgtatagcggcctggcgctggcgggcctgtatattctggaaacccatcgcagcgaacgcgcgagcccgctggaaacctatcgcccgcatgaaagcgaacgcctggaaattctggaagcgagcaacctgtatagcgcgctggcgctgtatagcacccatcgcgtggcgctgggcctgggcctgtatgtggcgctgggcctggcgctggcgctggaactgtatagcgcgagcaacggcctgattctggaactggaactgtatagcgcgctggcgcatattagcgcgctggcgggcctgtatggcctgggcctgacccatcgcccgcgcggcctgctggaactgtatagcgcgagcccgggcctgtatacctatcgcgcgctggcgacccatcgcacctatcgcggcctgggcctggcgctggcgggcctggcgctggcggcgctggcggcgctggcgctgtatagcggcctggcgctggcgctggaactgtatagcgcgagcaacgcgagcccggcgagcccggtggcgctggcgagcaacgcgagcaacgcgctggcgacctatcgcggcctgattctggaagtggcgctgggcctgaacggcctgtatgcgctggcggcgagcccgggcctgtatgcgcgcggcacctatcgcacctatcgcacctatcgcgtggcgctgctggaactgtatagcattctggaaggcctggtggcgctggcgctggcggcgagcccgggcctgggcctgggcctgccgcgcggcctgtatggcctggcgagcccgacccatcgcccgcgcggcctggtggcgctgggcctgaacggcctgggcctgtatacctatcgcgcgctggcgacccatcgcacctatcgcggcctgggcctggcgctggcgggcctggcgctggcggcgctggcggcgctggcgctgtatagcggcctggcgctggcgctggaactgtatagcggcctggcgagcccgctgtatagcgtggcgctggcgagcaacgcgagcaacgcgctggcgacctatcgcggcctggtggcgctggtggcgctgggcctgaacggcctgtatgcgctggcggcgagcccgggcctgtatgcgcgcggcacctatcgcacctatcgcacctatcgcgtggcgctgctggaactgtatagcattctggaaggcctggcgagcccgctgtatagcggcctggcgagcccgggcctgggcctgaacccgcgcggcctgtatggcctgggcctgccgcgcggcctgtatggcctggcgagcaacccgcgcggcctgtatattctggaaacccatcgcattctggaagcgagcccgggcctgacccgcccgctggaactggaactgtatagcgcgagcaacgcgctggcgctgtatagcggcctggcgagcccggcgctggcgattctggaactgtatagcggcctgctggaactgtatagcggcctggcgctggcgggcctgtatattctggaaagcgaacgcagcgaacgcgcgagcccgattctggaaacctatcgcccgcatgaagcgagcccggcgctggcgattctggaagcgagcaacctgtatagcgcgctggcgctgtatagcacccatcgcgtggcgctgggcctgggcctgtatgtggcgctgggcctggcgctggcgctggaactgtatagcgcgagcaacggcctgattctggaactggaactgtatagcgcgctggcgcatattagcgcgctggcgggcctgctgtatagcccgcgcggcctgtatggcctggcgagcaacccgcgcggcctgtatattctggaaacccatcgcattctggaagcgagcccgggcctgacccgcccgctggaactggaactgtatagcgcgagcaacgcgctggcgctgtatagcggcctggcgctggcggcgctggcgattctggaactgtatagcggcctgctggaactgtatagcggcctggcgctggcgggcctgtatattctggaaacccatcgcgcgctggcgggcctgacctatcgcctggaaccgcatgaagcgagcaacctggaaattctggaagcgagcaacctgtatagcgcgctggcgctgtatagcacccatcgcgtggcgctgggcctgggcctgtatgtggcgctgggcctgagcgaacgcctggaactgtatagcgcgagcaacggcctgattctggaactggaactgtatagcgcgctggcgcatattagcgcgctggcgggcctgctgtatagcccgcgcggcctgtatggcctggcgagcaacccgcgcggcctgtatattctggaaacccatcgcattctggaagcgagcccgggcctgacccgcccgctggaactggaactgtatagcgcgagcaacgcgctggcgctgtatagcggcctggcgagcccggcgctggcgattctggaactgtatagcggcctgctggaactgtatagcggcctggcgctggcgggcctgtatattctggaaacccatcgcagcgaacgcgcgagcccgattctggaaacctatcgcccgcatgaagcgagcccggcgctggcgattctggaagcgagcaacctgtatagcgcgctggcgctgtatagcacccatcgcattctggaaggcctgggcctgtatgtggcgctgggcctggcgctggcgctggaactgtatagcgcgagcaacggcctgattctggaactggaactgtatagcgcgctggcgcatattagcctgtatagcctgtatagcgcgagcccgggcctgggcctgccgcgcggcctgtatctgtatagcctgtatagcccgcgcggcctgtatggcctggcgagcccgctgtatagcctgtatagcccgcgcggcctggcgagcccgctgtatagcctgtatagcccgcgcggcctgtatggcctggcgagcccgctgtatagcctgtatagcccgcgcggcctggcgagcccgctgtatagcctgtatagcccgcgcggcctgtatggcctggcgagcccgctgtatagcctgtatagcccgcgcggcctggcgagcccgctgtatagcctgtatagcccgcgcggcctgtatctgtatagcacccatcgcgcgagcccgctgtatagcgcgagcccgagcgaacgcccgcgcgcgagcaacctgtatagcctgtatagcctgtatagcctgtatagcgcgctggcgctgtatagcctggaaccgcgcctgtatagcgcgctggcgggcctgtatagcgaacgcggcctggcgctggcgggcctgattctggaactggaaacccatcgcctggaagcgctggcggcgctggcggcgctggcggcgctggcgctggaaagcgaacgcacccatcgcgcgctggcggcgctggcgggcctgtatgcgctggcgacctatcgcgtggcgctgagcgaacgcctggaactgtatagcctgtatagcgcgcgcggcctgtatagc

SEQ ID NO: 60 - DNA, 129 bp, synthetic construct
  source 1..129: mol_type = other DNA
  misc_feature 73..75: note = NNN is a codon encoding a photoreactive crosslinker residue
ccgcatgaaggcctgggcctggcgctggcgacccatcgcgcgctggcgggcctggcgctggcgacctatcgcnnngcggcgacctatcgcgcgctggcggcgagcccgctggaactggaagcgctggcg

SEQ ID NO: 61 - DNA, 141 bp, synthetic construct
  source 1..141: mol_type = other DNA
  misc_feature 82..84: note = NNN is a codon encoding a photoreactive crosslinker residue
ccgcatgaagcgctggcgctgtatagcgcgctggcggtggcgctgagcgaacgcgcgagcccggcgctggcgacctatcgcnnngcggcgacctatcgcgcgctggcggcgagcccggcgctggcgctggaactgtatagc

SEQ ID NO: 62 - DNA, 132 bp, synthetic construct
  source 1..132: mol_type = other DNA
  misc_binding 76..78: bound_moiety = NNN is a codon encoding a photoreactive crosslinker residue
ccgcatgaaggcctgggcctggcgctggcgacccatcgcgcgctggcgctgtatagcgcgctggcgacctatcgcnnngcggcgacctatcgcgcgctggcggcgagcccgctggaactggaagcgctggcg

SEQ ID NO: 63 - DNA, 132 bp, synthetic construct
  source 1..132: mol_type = other DNA
  misc_feature 76..78: note = NNN is a codon encoding a photoreactive crosslinker residue
ccgcatgaaggcctgggcctggcgctggcgacccatcgcgcgctggcgctgtatagcgcgctggcgacctatcgcnnngcggcgacctatcgcgcgctggcggcgagcaacctggaactggaagcgctggcg

SEQ ID NO: 64 - DNA, 135 bp, synthetic construct
  source 1..135: mol_type = other DNA
  misc_feature 76..78: note = NNN is a codon encoding a photoreactive crosslinker residue
ccgcatgaaggcctgctgtatagcgcgctggcgacccatcgcagcgaacgcggcctggcgctggcgacctatcgcnnngcggcgacctatcgcgcgctggcggcgagcccgacccatcgcctggaactgtatagc

SEQ ID NO: 65 - DNA, 132 bp, synthetic construct
  source 1..132: mol_type = other DNA
  misc_feature 73..75: note = NNN is a codon encoding a photoreactive crosslinker residue
ccgcatgaaggcctgggcctggcgctggcgacccatcgcgcgctggcgggcctggcgctggcgacctatcgcnnngcggcgacctatcgcgcgctggcggcgagcccggcgctggcgctggaactgtatagc

SEQ ID NO: 66 - DNA, 132 bp, synthetic construct
  source 1..132: mol_type = other DNA
  misc_feature 76..78: note = NNN is a codon encoding a photoreative crosslinker residue
ccgcatgaagcgctggcgggcctggcgctggcgacccatcgcgcgctggcgggcctggcgctggcgacctatcgcnnngcggcgacctatcgcgcgctggcggcgagcccgctggaactggaagcgctggcg

SEQ ID NO: 67 - DNA, 132 bp, synthetic construct
  source 1..132: mol_type = other DNA
  misc_feature 76..78: note = NNN is a codon encoding a photoreative crosslinker residue
ccgcatgaagcgctggcgggcctggcgctggcgacccatcgcgcgctggcgggcctggcgctggcgacctatcgcnnngcggcgacctatcgcgcgctggcggcgagcccgctggaactggaaagcgaacgc

SEQ ID NO: 68 - DNA, 72 bp, synthetic construct
  source 1..72: mol_type = other DNA
gcgagcccgacctatcgcctgtatagcgcgagcccggcgagcccggcgagcccggcgagcccgctgtatagc

SEQ ID NO: 69 - DNA, 180 bp, synthetic construct
  source 1..180: mol_type = other DNA
ggcctgaacggcctgtatggcctgaacagcgaacgcggcctgtatggcctgaactgctatagcattctggaaagcgaacgcccgcgcgcgcgcggcggcctgtattgctatagcccgcgcgcgagcccgggcctgtatccgcgcacctatcgcgtggcgctgatggaaaccacctatcgc

SEQ ID NO: 70 - DNA, 36 bp, synthetic construct
  source 1..36: mol_type = other DNA
ctggaaccgcgcggcctgacccatcgcggcctgtat

SEQ ID NO: 71 - DNA, 501 bp, synthetic construct
  source 1..501: mol_type = other DNA
  misc_feature 487..501: note = codons encoding the Sortase A recognition site
ggcctgtatggcctgtatggcctgtatggcctgtatagcgaacgcggcctggcgctggcggcgctggcggcgctggcgctgtatagcggcctggcgctggcggcgctggcggcgctggcgctgtatagcggcctggcgctggcggcgctggcggcgctggcgctgtatagcggcctggcgctggcggcgctggcggcgctggcgctgtatagcggcctgtatggcctgtatggcctgtatggcctgtatagcgaacgcggcctggcgctggcggcgctggcggcgctggcgctgtatagcggcctggcgctggcggcgctggcggcgctggcgctgtatagcggcctggcgctggcggcgctggcggcgctggcgctgtatagcggcctggcgctggcggcgctggcggcgctggcgctgtatagcggcctgtatggcctgtatggcctgtatggcctgtatagcgaacgcccgcgcggcctgacccatcgcggcctgtat

SEQ ID NO: 72 - DNA, 471 bp, synthetic construct
  source 1..471: mol_type = other DNA
ggcctgtatggcctgtatggcctgtatggcctgtatagcgaacgcggcctggcgctggcggcgctggcggcgctggcgctgtatagcggcctggcgctggcggcgctggcggcgctggcgctgtatagcggcctggcgctggcggcgctggcggcgctggcgctgtatagcggcctggcgctggcggcgctggcggcgctggcgctgtatagcggcctgtatggcctgtatggcctgtatggcctgtatagcgaacgcggcctggcgctggcggcgctggcggcgctggcgctgtatagcggcctggcgctggcggcgctggcggcgctggcgctgtatagcggcctggcgctggcggcgctggcggcgctggcgctgtatagcggcctggcgctggcggcgctggcggcgctggcgctgtatagcggcctgtatggcctgtatggcctgtatggcctgtatagcgaacgc

SEQ ID NO: 73 - DNA, 453 bp, synthetic construct
  source 1..453: mol_type = other DNA
  misc_feature 439..453: note = Codons encoding Sortase A recognition site
ggcctgtatggcctgtatagcgaacgcggcctggcgctggcggcgctggcggcgctggcgctgtatagcggcctggcgctggcggcgctggcggcgctggcgctgtatagcggcctggcgctggcggcgctggcggcgctggcgctgtatagcggcctggcgctggcggcgctggcggcgctggcgctgtatagcggcctgtatggcctgtatagcgaacgcggcctggcgctggcggcgctggcggcgctggcgctgtatagcggcctggcgctggcggcgctggcggcgctggcgctgtatagcggcctggcgctggcggcgctggcggcgctggcgctgtatagcggcctggcgctggcggcgctggcggcgctggcgctgtatagcggcctgtatggcctgtatagcgaacgcctggaaccgcgcggcctgacccatcgcggcctgtat

SEQ ID NO: 74 - DNA, 417 bp, synthetic construct
  source 1..417: mol_type = other DNA
ggcctgtatggcctgtatagcgaacgcggcctggcgctggcggcgctggcggcgctggcgctgtatagcggcctggcgctggcggcgctggcggcgctggcgctgtatagcggcctggcgctggcggcgctggcggcgctggcgctgtatagcggcctggcgctggcggcgctggcggcgctggcgctgtatagcggcctgtatggcctgtatagcgaacgcggcctggcgctggcggcgctggcggcgctggcgctgtatagcggcctggcgctggcggcgctggcggcgctggcgctgtatagcggcctggcgctggcggcgctggcggcgctggcgctgtatagcggcctggcgctggcggcgctggcggcgctggcgctgtatagcggcctgtatggcctgtatagcgaacgc

SEQ ID NO: 75 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 214 aa, synthetic construct; identical to SEQ ID NO: 76

SEQ ID NO: 79 - DNA, 3771 bp, synthetic construct
  source 1..3771: mol_type = other DNA
ggcctgaacgtggcgctgggcctgaacctggaactgtatagcggcctgaacagcgaacgcggcctgtatccgcgcggcctgtatctggaagtggcgctgggcctgaacccgcgcagcgaacgcggcctgaacagcgaacgcctggaaagcgaacgcattctggaaacccatcgctgctatagcacccatcgcgtggcgctgagcgaacgcggcctgtatccgcatgaaagcgaacgcctggaaacccatcgcgcgagcaacacctatcgcggcctgtatgtggcgctgcatattagcacccgcccggtggcgctggcgcgcggcggcctgaacagcgaacgcccgcgcggcctgtatctgtatagcggcctgtatctggaaggcctgacccgcccgctggaaggcctgtatgtggcgctgattctggaaacccgcccgagcgaacgcggcctgtatggcctgtatgcgagcaacacccatcgcgcgagcccgacctatcgcgcgagcaacacccatcgcccgcgcccgcatgaaacccatcgcagcgaacgcgcgcgcggcctggaaagcgaacgcattctggaagcgagcaacctgtatagcgcgagcccggcgagcaacagcgaacgcctgtatagcagcgaacgcggcctgaacgtggcgctgccgcatgaaccgcatgaactgtatagcatggaaaccgcgagcaacagcgaacgcctggaaggcctgaacagcgaacgcgcgagcaacgcgagcccgacccatcgcgcgctggcgattctggaaacctatcgcacctatcgctgctatagcgcgctggcggcgcgcggcgcgctggcgctggaaacccatcgcacctatcgcacctatcgcgcgagcccgacctatcgcggcctgccgcatgaagcgctggcgacctatcgcacccgcccgggcctgtatggcctgaacggcctgtatacccatcgcctggaagtggcgctgacccatcgcgtggcgctgagcgaacgcgcgctggcggcgctggcgagcgaacgcacccatcgcctgtatagcggcctgtatccgcgcagcgaacgcgtggcgctgccgcatgaaccgcgcctggaagcgctggcgccgcgcagcgaacgcagcgaacgcctgtatagcagcgaacgcacccatcgcagcgaacgcggcctgtatggcctgtatacccatcgcgcgctggcggcgctggcgctggaaggcctgtattgctatagcctggaagtggcgctgctgtatagcgcgagcccgacctatcgcccgcatgaaccgcgcggcctgccgcgcgtggcgctgacccatcgcgtggcgctgagcgaacgcacccgcccggcgagcaacagcgaacgcggcctgtatgcgctggcgctggaaacccatcgcagcgaacgcggcctgtatgtggcgctgcatattagcacccatcgcccgcatgaaccgcgcgcgctggcggtggcgctgctggaaggcctgaacagcgaacgcagcgaacgcggcctgtatctggaaacctatcgcagcgaacgcctggaaagcgaacgcagcgaacgcgtggcgctggtggcgctgacccatcgcgtggcgctgccgcgcagcgaacgcagcgaacgcagcgaacgcctggaaggcctgtatacccatcgcggcctgaacacccatcgcacctatcgcattctggaatgctatagcgcgagcaacgtggcgctggcgagcaaccatattagcctgtatagcccgcgcagcgaacgcgcgagcaacacccatcgcctgtatagcgtggcgctggcgagcccgctgtatagcgcgcgcggcgtggcgctgggcctgccgcgcctgtatagcagcgaacgctgctatagcgcgagcccgctgtatagcacccatcgccatattagcacccatcgctgctatagcccgcgcccgcgctgctatagcccgcgcgcgctggcgccgcgcggcctgctggaactggaaggcctgtatggcctgtatccgcgcagcgaacgcgtggcgctgccgcatgaactggaaccgcatgaaccgcgcccgcgcctgtatagcccgcgcctgtatagcgcgagcccgacccatcgcctggaaatggaaaccattctggaaagcgaacgcgcgcgcggcacccatcgcccgcgcggcctggtggcgctgacccatcgctgctatagcgtggcgctggtggcgctggtggcgctggcgagcccggtggcgctgagcgaacgccatattagcggcctggcgagcccgccgcgcggcctggtggcgctgctgtatagcccgcatgaagcgagcaacacccgcccgacctatcgcgtggcgctggcgagcccgggcctgtatgtggcgctgggcctggtggcgctgcatattagcgcgagcaacgcgctggcgctgtatagcacccatcgcctgtatagcccgcgcgcgcgcggcggcctgggcctgggcctgaacacctatcgcgcgagcaacagcgaacgcacccatcgcacctatcgcgcgcgcggcgtggcgctggtggcgctgagcgaacgcgtggcgctgctggaaacccatcgcgtggcgctgctggaacatattagcggcctgaacgcgagcccgacccgcccgctggaagcgagcaacggcctgtatctgtatagcggcctgacctatcgcctgtatagctgctatagcctgtatagcgtggcgctgagcgaacgcgcgagcaacctgtatagcgcgctggcgctggaaccgcgcgcgctggcgccgcgcattctggaaggcctgctgtatagcacccatcgcattctggaaagcgaacgcctgtatagcgcgctggcgctgtatagcggcctgtatggcctgaacccgcgcgcgcgcggcggcctgccgcgcggcctgaacgtggcgctgacctatcgcacccatcgcctggaaccgcgcccgcgcagcgaacgcgcgcgcggcggcctgggcctgatggaaaccacccatcgcctgtatagcgcgagcaacggcctgaacgtggcgctgagcgaacgcctggaaacccatcgctgctatagcctggaagtggcgctgctgtatagcggcctgtatccgcatgaaacctatcgcccgcgcagcgaacgcgcgagcccgattctggaagcgctggcggtggcgctgggcctgacccgcccgggcctgagcgaacgcgcgagcaacggcctgtatggcctgaacccgcgcggcctggcgagcaacgcgagcaacacctatcgcctgtatagcacccatcgcacccatcgcccgcgcccgcgcgtggcgctgctggaagcgagcccgagcgaacgcgcgagcccgggcctgtatagcgaacgcccgcatgaaccgcatgaactggaaacctatcgcagcgaacgcctgtatagcctggaaacccatcgcgtggcgctggcgagcccgctgtatagcagcgaacgcgcgcgcggcacccgcccgggcctgaacggcctgaacggcctgtatgcgagcaacgtggcgctgccgcatgaaagcgaacgctgctatagcagcgaacgcgtggcgctgatggaaacccatattagcggcctggcgctggcgctggaacatattagcgcgagcaaccatattagcacctatcgcacccatcgcggcctgaacctgtatagcagcgaacgcctggaaagcgaacgcctggaaagcgaacgcccgcgcggcctgtatctgtatagc

SEQ ID NO: 80 - DNA, 1812 bp, synthetic construct
  source 1..1812: mol_type = other DNA
gcgagcccgattctggaactggaactggaaacccatcgcggcctgaacagcgaacgcccgcgcgtggcgctgattctggaactggaaagcgaacgcgtggcgctgagcgaacgcccgcgcggcctgtatggcctggcgcgcggcgtggcgctgagcgaacgcccgcatgaaagcgaacgctgctatagcgcgcgcggcgcgctggcgagcgaacgcggcctgaacagcgaacgcattctggaaggcctgtatacccatcgcgcgagcaacattctggaacatattagcacccgcccgacctatcgcggcctgaacggcctgaacgcgcgcggcacccatcgcgcgagcaacggcctgtatagcgaacgcccgcgcgcgcgcggcctggaactggaaattctggaactgtatagcacctatcgcgcgctggcgagcgaacgcggcctgagcgaacgcattctggaaagcgaacgcggcctgtatattctggaaccgcgcagcgaacgcgcgcgcggcccgcatgaaagcgaacgcggcctgtatagcgaacgcggcctgtatagcgaacgcggcctgtatacccatcgcgcgagcccgccgcatgaaacccatcgcctggaaagcgaacgcattctggaagcgagcaacagcgaacgcgtggcgctgggcctgagcgaacgcggcctggcgagcccgattctggaagcgctggcggcgagcccgacctatcgcacctatcgctgctatagcggcctgaacggcctgaacgcgagcaacgcgagcaacgcgagcaacacccgcccgccgcgcacccatcgcacccatcgcccgcatgaaggcctgtatgcgctggcgggcctgtatacccatcgcctgtatagcctggaaggcctgctggaactgtatagcgcgcgcggcacccatcgcgtggcgctggcgctggcggcgctggcgccgcgcagcgaacgcgtggcgctgccgcatgaaattctggaaccgcatgaaccgcgcccgcgcagcgaacgcgcgagcccgggcctgggcctgaacctggaactgtatagcagcgaacgcggcctgtatacccatcgcgcgctggcgagcgaacgcgtggcgctggtggcgctgtgctatagcctggaactggaagcgagcaacgcgagcaacccgcatgaaacctatcgcccgcgcgcgcgcggcggcctggcgctggcgctgtatagcgtggcgctgggcctgaacacccgcccgctgtatagcgtggcgctggcgagcccggcgagcaacgcgctggcgctggaaggcctgaacagcgaacgcggcctgtatgcgagcaacagcgaacgcggcctgaacggcctgagcgaacgcgtggcgctgacccatcgcggcctgggcctgaacgcgagcccgagcgaacgcctgtatagcgcgagcccgagcgaacgcacccatcgcacctatcgcagcgaacgcctggaaagcgaacgcagcgaacgcacccatcgcctggaaacccatcgcctggaaagcgaacgcctgtatagcgcgctggcggcgagcccgacctatcgcggcctgctgtatagccatattagcctgtatagcgtggcgctgacctatcgcgcgctggcgtgctatagcggcctggtggcgctgacccatcgccatattagcggcctgaacggcctgtatctggaaagcgaacgcagcgaacgcccgcgcgtggcgctgacccatcgcctgtatagcagcgaacgcccgcatgaagcgagcaacgcgcgcggcggcctgtatggcctgtgctatagc

SEQ ID NO: 81 - DNA, 3771 bp, synthetic construct
  source 1..3771: mol_type = other DNA
ggcctgaacgtggcgctgggcctgaacctggaactgtatagcggcctgaacagcgaacgcggcctgtatccgcgcggcctgtatctggaagtggcgctgggcctgaacccgcgcagcgaacgcggcctgaacagcgaacgcctggaaagcgaacgcattctggaaacccatcgctgctatagcacccatcgcgtggcgctgagcgaacgcggcctgtatccgcatgaaagcgaacgcctggaaacccatcgcgcgagcaacacctatcgcggcctgtatgtggcgctgcatattagcacccgcccggtggcgctggcgcgcggcggcctgaacagcgaacgcccgcgcggcctgtatctgtatagcggcctgtatctggaaggcctgacccgcccgctggaaggcctgtatgtggcgctgattctggaaacccgcccgagcgaacgcggcctgtatggcctgtatgcgagcaacacccatcgcgcgagcccgacctatcgcgcgagcaacacccatcgcccgcgcccgcatgaaacccatcgcagcgaacgcgcgcgcggcctggaaagcgaacgcattctggaagcgagcaacctgtatagcgcgagcccggcgagcaacagcgaacgcctgtatagcagcgaacgcggcctgaacgtggcgctgccgcatgaaccgcatgaactgtatagcatggaaaccgcgagcaacagcgaacgcctggaaggcctgaacagcgaacgcgcgagcaacgcgagcccgacccatcgcgcgctggcgattctggaaacctatcgcacctatcgctgctatagcgcgctggcggcgcgcggcgcgctggcgctggaaacccatcgcacctatcgcacctatcgcgcgagcccgacctatcgcggcctgccgcatgaagcgctggcgacctatcgcacccgcccgggcctgtatggcctgaacggcctgtatacccatcgcctggaagtggcgctgacccatcgcgtggcgctgagcgaacgcgcgctggcggcgctggcgagcgaacgcacccatcgcctgtatagcggcctgtatccgcgcagcgaacgcgtggcgctgccgcatgaaccgcgcctggaagcgctggcgccgcgcagcgaacgcagcgaacgcctgtatagcagcgaacgcacccatcgcagcgaacgcggcctgtatggcctgtatacccatcgcgcgctggcggcgctggcgctggaaggcctgtattgctatagcctggaagtggcgctgctgtatagcgcgagcccgacctatcgcccgcatgaaccgcgcggcctgccgcgcgtggcgctgacccatcgcgtggcgctgagcgaacgcacccgcccggcgagcaacagcgaacgcggcctgtatgcgctggcgctggaaacccatcgcagcgaacgcggcctgtatgtggcgctgcatattagcacccatcgcccgcatgaaccgcgcgcgctggcggtggcgctgctggaaggcctgaacagcgaacgcagcgaacgcggcctgtatctggaaacctatcgcagcgaacgcctggaaagcgaacgcagcgaacgcgtggcgctggtggcgctgacccatcgcgtggcgctgccgcgcagcgaacgcagcgaacgcagcgaacgcctggaaggcctgtatacccatcgcggcctgaacacccatcgcacctatcgcattctggaatgctatagcgcgagcaacgtggcgctggcgagcaaccatattagcctgtatagcccgcgcagcgaacgcgcgagcaacacccatcgcctgtatagcgtggcgctggcgagcccgctgtatagcctgtatagcgtggcgctgggcctgccgcgcctgtatagcagcgaacgctgctatagcgcgagcccgctgtatagcacccatcgccatattagcacccatcgctgctatagcccgcgcccgcgctgctatagcccgcgcgcgctggcgccgcgcggcctgctggaactggaaggcctgtatggcctgtatccgcgcagcgaacgcgtggcgctgccgcatgaactggaaccgcatgaaccgcgcccgcgcctgtatagcccgcgcctgtatagcgcgagcccgacccatcgcctggaaatggaaaccattctggaaagcgaacgcgcgcgcggcacccatcgcccgcgcggcctggtggcgctgacccatcgctgctatagcgtggcgctggtggcgctggtggcgctggcgagcccggtggcgctgagcgaacgccatattagcggcctggcgagcccgccgcgcggcctggtggcgctgctgtatagcccgcatgaagcgagcaacacccgcccgacctatcgcgtggcgctggcgagcccgggcctgtatgtggcgctgggcctggtggcgctgcatattagcgcgagcaacgcgctggcgctgtatagcacccatcgcctgtatagcccgcgcgcgcgcggcggcctgggcctgggcctgaacacctatcgcgcgagcaacagcgaacgcacccatcgcacctatcgcgcgcgcggcgtggcgctggtggcgctgagcgaacgcgtggcgctgctggaaacccatcgcgtggcgctgctggaacatattagcggcctgaacgcgagcccgacccgcccgctggaagcgagcaacggcctgtatctgtatagcggcctgacctatcgcctgtatagctgctatagcctgtatagcgtggcgctgagcgaacgcgcgagcaacctgtatagcgcgctggcgctggaaccgcgcgcgctggcgccgcgcattctggaaggcctgctgtatagcacccatcgcattctggaaagcgaacgcctgtatagcgcgctggcgctgtatagcggcctgtatggcctgaacccgcgcgcgcgcggcggcctgccgcgcggcctgaacgtggcgctgacctatcgcacccatcgcctggaaccgcgcccgcgcagcgaacgcgcgcgcggcgcgagcccgggcctgctggaaacccatcgcctgtatagcgcgagcaacggcctgaacgtggcgctgagcgaacgcctggaaacccatcgctgctatagcctggaagtggcgctgctgtatagcggcctgtatccgcatgaaacctatcgcccgcgcagcgaacgcgcgagcccgattctggaagcgctggcggtggcgctgggcctgacccgcccgggcctgagcgaacgcgcgagcaacggcctgtatggcctgaacccgcgcggcctggcgagcaacgcgagcaacacctatcgcctgtatagcacccatcgcacccatcgcccgcgcccgcgcgtggcgctgctggaagcgagcccgagcgaacgcgcgagcccgggcctgtatagcgaacgcccgcatgaaccgcatgaactggaaacctatcgcagcgaacgcctgtatagcctggaaacccatcgcgtggcgctggcgagcccgctgtatagcagcgaacgcgcgcgcggcacccgcccgggcctgaacggcctgaacggcctgtatgcgagcaacgtggcgctgccgcatgaaagcgaacgctgctatagcagcgaacgcgtggcgctgatggaaacccatattagcggcctggcgctggcgctggaacatattagcgcgagcaaccatattagcacctatcgcacccatcgcggcctgaacctgtatagcagcgaacgcctggaaagcgaacgcctggaaagcgaacgcccgcgcggcctgtatctgtatagc

SEQ ID NO: 82 - DNA, 1812 bp, synthetic construct; identical to SEQ ID NO: 80
  source 1..1812: mol_type = other DNA

SEQ ID NO: 83 - protein, 6 aa, synthetic construct
GGSGGS

SEQ ID NO: 84 - protein, 5 aa, synthetic construct
GGGGS

SEQ ID NO: 85 - protein, 20 aa, synthetic construct
EAAAKEAAAKEAAAKEAAAK

SEQ ID NO: 86 - protein, 5 aa, synthetic construct
SSSSS

SEQ ID NO: 87 - protein, 5 aa, synthetic construct
EAAAK

SEQ ID NO: 88 - protein, 8 aa, synthetic construct
GPLGIAGQ

SEQ ID NO: 89 - protein, 8 aa, synthetic construct
AVRWLLTA

SEQ ID NO: 90 - protein, 6 aa, synthetic construct
RRRRRR